SEQUENCE LISTING (54 sequences)

SEQ ID NO: 1 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 135 aa, Homo sapiens
MVLGTIDLCSCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - DNA, 23 bp, synthetic construct
  source 1..23: mol_type = other DNA
ggccgagatgtctcgctccgtgg

SEQ ID NO: 4 - DNA, 23 bp, synthetic construct
  source 1..23: mol_type = other DNA
ggccacggagcgagacatctcgg

SEQ ID NO: 5 - DNA, 2201 bp, synthetic construct
  source 1..2201: mol_type = other DNA
aaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggcgggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatgcgcataagtaagcctcacttgagatctattagcatacagtgttacctgtgccttctcctcaattctcacttcctgacagaagccggaatccacgtttttattctcggttgcttcagtgccggactgcctaagacggaagctaattgggtcaacgttataagcgatctcaaaaaaatcgaagatttgatacagagcatgcacattgatgctactttgtatacagaaagtgacgtacacccaagttgtaaagtaacggcgatgaaatgtttcctgctcgagctgcaggtaatcagcctcgagtctggtgacgcctcaatacatgacaccgttgagaatttgataatccttgcgaataactccttgtcttcaaatggcaacgttacggaaagcgggtgcaaagaatgcgaagagttggaggagaaaaacattaaggaattccttcagagttttgtacacatagtgcaaatgttcattaatacaagcggatctggagcaacaaacttctcactactcaaacaagcaggtgacgtggaggagaatcccggccccatgtctcgctccgtggccttagctgtgctcgcgctactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagata

SEQ ID NO: 6 - DNA, 23 bp, synthetic construct
  source 1..23: mol_type = other DNA
tgctggcgatacgcgtccacagg

SEQ ID NO: 7 - DNA, 23 bp, synthetic construct
  source 1..23: mol_type = other DNA
aacgtgcggtgggatcgtgctgg

SEQ ID NO: 8 - DNA, 23 bp, synthetic construct
  source 1..23: mol_type = other DNA
atgatagtcactgacaacaacgg

SEQ ID NO: 9 - DNA, 23 bp, synthetic construct
  source 1..23: mol_type = other DNA
agttgctcatgcaggatttctgg

SEQ ID NO: 10 - DNA, 23 bp, synthetic construct
  source 1..23: mol_type = other DNA
gaagccacaggaagtctgtgtgg

SEQ ID NO: 11 - DNA, 23 bp, synthetic construct
  source 1..23: mol_type = other DNA
ccgacttctgaacgtgcggtggg

SEQ ID NO: 12 - DNA, 23 bp, synthetic construct
  source 1..23: mol_type = other DNA
tatcatgtcgttattaactgagg

SEQ ID NO: 13 - DNA, 23 bp, synthetic construct
  source 1..23: mol_type = other DNA
tctactgctaccgcgttaaccgg

SEQ ID NO: 14 - DNA, 23 bp, synthetic construct
  source 1..23: mol_type = other DNA
gcttctgctgccggttaacgcgg

SEQ ID NO: 15 - DNA, 88 bp, synthetic construct
  source 1..88: mol_type = other DNA
gagttgccatatctgtcatcatcatcttctactgctgatgatgataccgcgttaaccggcagcagaagctgagttcaacctgggaaac

SEQ ID NO: 16 - DNA, 87 bp, synthetic construct
  source 1..87: mol_type = other DNA
gagttgccatatctgtcatcatcatcttctactgctgatgatgataccgggttaaccggcagcagaagctgagttcaacctgggaaa

SEQ ID NO: 17-24 - skipped (8 entries)

SEQ ID NO: 25 - DNA, 1682 bp, synthetic construct
  source 1..1682: mol_type = other DNA
tacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctggatctggagctactaacttcagcctgctgaagcaggctggagacgtggaggagaaccctggacccatgtctcgctccgtggcccttgctgtcctcgccttactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttc

SEQ ID NO: 26 - DNA, 2927 bp, synthetic construct
  source 1..2927: mol_type = other DNA
acagccaacctttcatcacctcttcttccagacctgagattgcctcaagagaacaaagctcctggtctcactatggggtgctgaggaggtgtcggttaaatgactactaagcggcaaatccctcttcttgatacaagaaagagtttccagatttattatattaaagatcattttatgatctttacatttatcatgatcattttataatcatgatacgtttatatacccacataaacgtacacatacatgcagagaacacccctagaaactacatttaataatcgaaagagagatggtctaaaggaaagggaaatggaacaggtgtttacatttaggagacagagatacactgactgtgtgtactatgagaatacattatgtaaaaaggggaaaagaaagaataacttcttaaaaggcttgcttaccatacagccacacagacttcctgtggcttctcacagatggaggtgatgctgcagttgctcatgcaggatttctggggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattaggatccatcgccaccatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgaatccctaggcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcacaggtggaaaatctcacatcacaaaatttacacagttgtggaaacttgactgcaccgttgttgtcagtgactatcatgtcgttattaactgaggagagagaaagatatattaaatgattatccaactgccaggcagcctgccaatgaattcctgaagatgttatgcaatttcaaatgaacttgatgtcatgagaatgaatctgaagaaaggcaaaataattccttcacatcagattagaattatctggtgtatgcaatttgtaataaaatccattgtaccgtggtgaggggtggtgaggggttggggtggttgagtagccatttgcttttgtcaatggttgtcataaggtccactttctacaaaaatcttatcattcaataaagaaacctagatgggcccactgcatagtacggagtgctgggcccacaaataaaagtacccttaaggaaattatagtttagctggtgaataagacaggaaacagaccaagtcagggt

SEQ ID NO: 27 - DNA, 2460 bp, synthetic construct
  source 1..2460: mol_type = other DNA
gcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatggccttaccagtgaccgccttgctcctgccgctggccttgctgctccacgccgccaggccgaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggaggatcaggtggcggtggaagcggaggtggaggctccggtggaggaggtagtggcggaggttctcttcaaatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctaggatctggagcaacaaacttctcactactcaaacaagctggagacgtggaggagaatcccggccccatgtctcgctccgtggcccttgctgtcctcgccttactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttc

SEQ ID NO: 28 - DNA, 1878 bp, synthetic construct
  source 1..1878: mol_type = other DNA
gcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatggccttaccagtgaccgccttgctcctgccgctggccttgctgctccacgccgccaggccgaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcggatctggagcaacaaacttctcactactcaaacaagctggagacgtggaggagaatcccggccccatgtctcgctccgtggcccttgctgtcctcgccttactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttc

SEQ ID NO: 29 - DNA, 4542 bp, synthetic construct
  source 1..4542: mol_type = other DNA
tatatgaataatataaccagaatttctgactaaaaaaacaggttgtttttgtggttctccaagtagattccccatttttggctggtagttttctcttccaacagccaacctttcatcacctcttcttccagacctgagattgcctcaagagaacaaagctcctggtctcactatggggtgctgaggaggtgtcggttaaatgactactaagcggcaaatccctcttcttgatacaagaaagagtttccagatttattatattaaagatcattttatgatctttacatttatcatgatcattttataatcatgatacgtttatatacccacataaacgtacacatacatgcagagaacacccctagaaactacatttaataatcgaaagagagatggtctaaaggaaagggaaatggaacaggtgtttacatttaggagacagagatacactgactgtgtgtactatgagaatacattatgtaaaaaggggaaaagaaagaataacttcttaaaaggcttgcttaccatacagccacacagacttcctgtggcttctcacagatggaggtgatgctgcagttgctcatgcaggatttctggtcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctcatcgatactagtgtacaggttaatgatgttggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattaggatccatcgccaccatggccttaccagtgaccgccttgctcctgccgctggccttgctgctccacgccgccaggccgaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggaggatcaggtggcggtggaagcggaggtggaggctccggtggaggaggtagtggcggaggttctcttcaaatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctatgaatccctaggcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcaggtacctcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccacaggtggaaaatctcacatcacaaaatttacacagttgtggaaacttgactgcaccgttgttgtcagtgactatcatgtcgttattaactgaggagagagaaagatatattaaatgattatccaactgccaggcagcctgccaatgaattcctgaagatgttatgcaatttcaaatgaacttgatgtcatgagaatgaatctgaagaaaggcaaaataattccttcacatcagattagaattatctggtgtatgcaatttgtaataaaatccattgtaccgtggtgaggggtggtgaggggttggggtggttgagtagccatttgcttttgtcaatggttgtcataaggtccactttctacaaaaatcttatcattcaataaagaaacctagatgggcccactgcatagtacggagtgctgggcccacaaataaaagtacccttaaggaaattatagtttagctggtgaataagacaggaaacagaccaagtcagggtgacccaaaatagagggcagcacagaggatgtgggagccaggcgagcatcaccgctcagctgggcagggaggctataacctcacagagtcatgaagtgcca

SEQ ID NO: 30 - DNA, 3960 bp, synthetic construct
  source 1..3960: mol_type = other DNA
tatatgaataatataaccagaatttctgactaaaaaaacaggttgtttttgtggttctccaagtagattccccatttttggctggtagttttctcttccaacagccaacctttcatcacctcttcttccagacctgagattgcctcaagagaacaaagctcctggtctcactatggggtgctgaggaggtgtcggttaaatgactactaagcggcaaatccctcttcttgatacaagaaagagtttccagatttattatattaaagatcattttatgatctttacatttatcatgatcattttataatcatgatacgtttatatacccacataaacgtacacatacatgcagagaacacccctagaaactacatttaataatcgaaagagagatggtctaaaggaaagggaaatggaacaggtgtttacatttaggagacagagatacactgactgtgtgtactatgagaatacattatgtaaaaaggggaaaagaaagaataacttcttaaaaggcttgcttaccatacagccacacagacttcctgtggcttctcacagatggaggtgatgctgcagttgctcatgcaggatttctggtcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctcatcgatactagtgtacaggttaatgatgttggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattaggatccatcgccaccatggccttaccagtgaccgccttgctcctgccgctggccttgctgctccacgccgccaggccgaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgctgaatccctaggcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcaggtacctcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccacaggtggaaaatctcacatcacaaaatttacacagttgtggaaacttgactgcaccgttgttgtcagtgactatcatgtcgttattaactgaggagagagaaagatatattaaatgattatccaactgccaggcagcctgccaatgaattcctgaagatgttatgcaatttcaaatgaacttgatgtcatgagaatgaatctgaagaaaggcaaaataattccttcacatcagattagaattatctggtgtatgcaatttgtaataaaatccattgtaccgtggtgaggggtggtgaggggttggggtggttgagtagccatttgcttttgtcaatggttgtcataaggtccactttctacaaaaatcttatcattcaataaagaaacctagatgggcccactgcatagtacggagtgctgggcccacaaataaaagtacccttaaggaaattatagtttagctggtgaataagacaggaaacagaccaagtcagggtgacccaaaatagagggcagcacagaggatgtgggagccaggcgagcatcaccgctcagctgggcagggaggctataacctcacagagtcatgaagtgcca

SEQ ID NO: 31 - DNA, 2481 bp, synthetic construct
  source 1..2481: mol_type = other DNA
gcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatggccttaccagtgaccgccttgctcctgccgctggccttgctgctccacgccgccaggccgaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggaggatcaggtggcggtggaagcggaggtggaggctccggtggaggaggtagtggcggaggttctcttcaaatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctaggctccgagaaggacgagctgggatctggagcaacaaacttctcactactcaaacaagctggagacgtggaggagaatcccggccccatgtctcgctccgtggcccttgctgtcctcgccttactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttc

SEQ ID NO: 32 - DNA, 1773 bp, synthetic construct
  source 1..1773: mol_type = other DNA
gcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctggctccgagaaggacgagctgggatctggagcaacaaacttctcactactcaaacaagctggagacgtggaggagaatcccggccccatgtctcgctccgtggcccttgctgtcctcgccttactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttc

SEQ ID NO: 33 - DNA, 2416 bp, synthetic construct
  source 1..2416: mol_type = other DNA
gcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatggccttaccagtgaccgccttgctcctgccgctggccttgctgctccacgccgccaggccgaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgctgaaattccgcccctctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataaccgccaccatgtctcgctccgtggcccttgctgtcctcgccttactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttc

SEQ ID NO: 34 - DNA, 2998 bp, synthetic construct
  source 1..2998: mol_type = other DNA
gcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatggccttaccagtgaccgccttgctcctgccgctggccttgctgctccacgccgccaggccgaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggaggatcaggtggcggtggaagcggaggtggaggctccggtggaggaggtagtggcggaggttctcttcaaatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctatgaaattccgcccctctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataaccgccaccatgtctcgctccgtggcccttgctgtcctcgccttactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttc

SEQ ID NO: 35 - DNA, 4563 bp, synthetic construct
  source 1..4563: mol_type = other DNA
tatatgaataatataaccagaatttctgactaaaaaaacaggttgtttttgtggttctccaagtagattccccatttttggctggtagttttctcttccaacagccaacctttcatcacctcttcttccagacctgagattgcctcaagagaacaaagctcctggtctcactatggggtgctgaggaggtgtcggttaaatgactactaagcggcaaatccctcttcttgatacaagaaagagtttccagatttattatattaaagatcattttatgatctttacatttatcatgatcattttataatcatgatacgtttatatacccacataaacgtacacatacatgcagagaacacccctagaaactacatttaataatcgaaagagagatggtctaaaggaaagggaaatggaacaggtgtttacatttaggagacagagatacactgactgtgtgtactatgagaatacattatgtaaaaaggggaaaagaaagaataacttcttaaaaggcttgcttaccatacagccacacagacttcctgtggcttctcacagatggaggtgatgctgcagttgctcatgcaggatttctggtcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctcatcgatactagtgtacaggttaatgatgttggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattaggatccatcgccaccatggccttaccagtgaccgccttgctcctgccgctggccttgctgctccacgccgccaggccgaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggaggatcaggtggcggtggaagcggaggtggaggctccggtggaggaggtagtggcggaggttctcttcaaatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctaggctccgagaaggacgagctgtgaatccctaggcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcaggtacctcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccacaggtggaaaatctcacatcacaaaatttacacagttgtggaaacttgactgcaccgttgttgtcagtgactatcatgtcgttattaactgaggagagagaaagatatattaaatgattatccaactgccaggcagcctgccaatgaattcctgaagatgttatgcaatttcaaatgaacttgatgtcatgagaatgaatctgaagaaaggcaaaataattccttcacatcagattagaattatctggtgtatgcaatttgtaataaaatccattgtaccgtggtgaggggtggtgaggggttggggtggttgagtagccatttgcttttgtcaatggttgtcataaggtccactttctacaaaaatcttatcattcaataaagaaacctagatgggcccactgcatagtacggagtgctgggcccacaaataaaagtacccttaaggaaattatagtttagctggtgaataagacaggaaacagaccaagtcagggtgacccaaaatagagggcagcacagaggatgtgggagccaggcgagcatcaccgctcagctgggcagggaggctataacctcacagagtcatgaagtgcca

SEQ ID NO: 36 - DNA, 3855 bp, synthetic construct
  source 1..3855: mol_type = other DNA
tatatgaataatataaccagaatttctgactaaaaaaacaggttgtttttgtggttctccaagtagattccccatttttggctggtagttttctcttccaacagccaacctttcatcacctcttcttccagacctgagattgcctcaagagaacaaagctcctggtctcactatggggtgctgaggaggtgtcggttaaatgactactaagcggcaaatccctcttcttgatacaagaaagagtttccagatttattatattaaagatcattttatgatctttacatttatcatgatcattttataatcatgatacgtttatatacccacataaacgtacacatacatgcagagaacacccctagaaactacatttaataatcgaaagagagatggtctaaaggaaagggaaatggaacaggtgtttacatttaggagacagagatacactgactgtgtgtactatgagaatacattatgtaaaaaggggaaaagaaagaataacttcttaaaaggcttgcttaccatacagccacacagacttcctgtggcttctcacagatggaggtgatgctgcagttgctcatgcaggatttctggtcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctcatcgatactagtgtacaggttaatgatgttggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattaggatccatcgccaccatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctggctccgagaaggacgagctgtgaatccctaggcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcaggtacctcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccacaggtggaaaatctcacatcacaaaatttacacagttgtggaaacttgactgcaccgttgttgtcagtgactatcatgtcgttattaactgaggagagagaaagatatattaaatgattatccaactgccaggcagcctgccaatgaattcctgaagatgttatgcaatttcaaatgaacttgatgtcatgagaatgaatctgaagaaaggcaaaataattccttcacatcagattagaattatctggtgtatgcaatttgtaataaaatccattgtaccgtggtgaggggtggtgaggggttggggtggttgagtagccatttgcttttgtcaatggttgtcataaggtccactttctacaaaaatcttatcattcaataaagaaacctagatgggcccactgcatagtacggagtgctgggcccacaaataaaagtacccttaaggaaattatagtttagctggtgaataagacaggaaacagaccaagtcagggtgacccaaaatagagggcagcacagaggatgtgggagccaggcgagcatcaccgctcagctgggcagggaggctataacctcacagagtcatgaagtgcca

SEQ ID NO: 37 - DNA, 3019 bp, synthetic construct
  source 1..3019: mol_type = other DNA
gcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatggccttaccagtgaccgccttgctcctgccgctggccttgctgctccacgccgccaggccgaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggaggatcaggtggcggtggaagcggaggtggaggctccggtggaggaggtagtggcggaggttctcttcaaatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctaggctccgagaaggacgagctgtgaaattccgcccctctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataaccgccaccatgtctcgctccgtggcccttgctgtcctcgccttactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttc

SEQ ID NO: 38 - DNA, 2311 bp, synthetic construct
  source 1..2311: mol_type = other DNA
gcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctggctccgagaaggacgagctgtgaaattccgcccctctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataaccgccaccatgtctcgctccgtggcccttgctgtcctcgccttactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttc

SEQ ID NO: 39 - DNA, 600 bp, synthetic construct
  source 1..600: mol_type = other DNA
gcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgag

SEQ ID NO: 40 - DNA, 63 bp, synthetic construct
  source 1..63: mol_type = other DNA
atggccttaccagtgaccgccttgctcctgccgctggccttgctgctccacgccgccaggccg

SEQ ID NO: 41 - DNA, 342 bp, synthetic construct
  source 1..342: mol_type = other DNA
aactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 42 - DNA, 78 bp, synthetic construct
  source 1..78: mol_type = other DNA
agcggcggaggatcaggtggcggtggaagcggaggtggaggctccggtggaggaggtagtggcggaggttctcttcaa

SEQ ID NO: 43 - DNA, 711 bp, synthetic construct
  source 1..711: mol_type = other DNA
atcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccaccta

SEQ ID NO: 44 - DNA, 57 bp, synthetic construct
  source 1..57: mol_type = other DNA
gcaacaaacttctcactactcaaacaagctggagacgtggaggagaatcccggcccc

SEQ ID NO: 45 - DNA, 600 bp, synthetic construct
  source 1..600: mol_type = other DNA
atgtctcgctccgtggcccttgctgtcctcgccttactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttc

SEQ ID NO: 46 - DNA, 63 bp, synthetic construct; identical to SEQ ID NO: 40
  source 1..63: mol_type = other DNA

SEQ ID NO: 47 - DNA, 525 bp, synthetic construct
  source 1..525: mol_type = other DNA
tatatgaataatataaccagaatttctgactaaaaaaacaggttgtttttgtggttctccaagtagattccccatttttggctggtagttttctcttccaacagccaacctttcatcacctcttcttccagacctgagattgcctcaagagaacaaagctcctggtctcactatggggtgctgaggaggtgtcggttaaatgactactaagcggcaaatccctcttcttgatacaagaaagagtttccagatttattatattaaagatcattttatgatctttacatttatcatgatcattttataatcatgatacgtttatatacccacataaacgtacacatacatgcagagaacacccctagaaactacatttaataatcgaaagagagatggtctaaaggaaagggaaatggaacaggtgtttacatttaggagacagagatacactgactgtgtgtactatgagaatacattatgtaaaaaggggaaaagaaagaataacttcttaaaaggcttgcttac

SEQ ID NO: 48 - DNA, 252 bp, synthetic construct
  source 1..252: mol_type = other DNA
tcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctc

SEQ ID NO: 49 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 50 - DNA, 21 bp, synthetic construct
  source 1..21: mol_type = other DNA
ggctccgagaaggacgagctg

SEQ ID NO: 51 - DNA, 228 bp, synthetic construct
  source 1..228: mol_type = other DNA
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 52 - DNA, 600 bp, synthetic construct
  source 1..600: mol_type = other DNA
cacaggtggaaaatctcacatcacaaaatttacacagttgtggaaacttgactgcaccgttgttgtcagtgactatcatgtcgttattaactgaggagagagaaagatatattaaatgattatccaactgccaggcagcctgccaatgaattcctgaagatgttatgcaatttcaaatgaacttgatgtcatgagaatgaatctgaagaaaggcaaaataattccttcacatcagattagaattatctggtgtatgcaatttgtaataaaatccattgtaccgtggtgaggggtggtgaggggttggggtggttgagtagccatttgcttttgtcaatggttgtcataaggtccactttctacaaaaatcttatcattcaataaagaaacctagatgggcccactgcatagtacggagtgctgggcccacaaataaaagtacccttaaggaaattatagtttagctggtgaataagacaggaaacagaccaagtcagggtgacccaaaatagagggcagcacagaggatgtgggagccaggcgagcatcaccgctcagctgggcagggaggctataacctcacagagtcatgaagtgcca

SEQ ID NO: 53 - DNA, 569 bp, synthetic construct
  source 1..569: mol_type = other DNA
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgat

SEQ ID NO: 54 - protein, 7 aa, synthetic construct
GSEKDEL

(19) United States
(12) Patent Application Publication
(10) Pub. No.: US 2026/0130993 A1
(43) Pub. Date: May 14, 2026

(54) NATURAL KILLER CELLS AND METHODS OF USE THEREOF

(71) Applicant: CYTOVIA THERAPEUTICS, LLC, Aventura, FL (US)

(72) Inventors: Anping CHEN, Aventura, FL (US); Hao-Ming CHANG, Aventura, FL (US); Wei LI, Aventura, FL (US)

(73) Assignee: CYTOVIA THERAPEUTICS, LLC, Aventura, FL (US)

(21) Appl. No.: 18/698,349

(22) PCT Filed: Oct. 5, 2022
(86) PCT No.: PCT/US2022/077621

§ 371 Date: Apr. 3, 2024

(51) Int. Cl. A61K 40/15; A61K 38/20; A61P 35/00; C12N 5/0783; C12N 15/90

(52) U.S. Cl. CPC A61K 40/15; A61K 38/2086; A61P 35/00; C12N 5/0646; C12N 15/907; C12N 2506/45

(57) ABSTRACT

The invention relates generally to populations of Natural Killer (NK) cells and methods of use thereof.

### CROSS-REFERENCE TO RELATED APPLICATIONS

This application is a U.S. national stage application under 35 U.S.C. 371 of PCT International Application No. PCT/US2022/077621, filed Oct. 5, 2022, which claims priority to U.S. Provisional Patent Application No. 63/252,487, filed Oct. 5, 2021, each of which is hereby incorporated by reference in its entirety.

### INCORPORATION-BY-REFERENCE OF SEQUENCE LISTING

The Sequence Listing XML associated with this application is provided electronically in XML file format and is hereby incorporated by reference into the specification. The name of the XML file containing the Sequence Listing XML is “P-626969-US_ST26.xml”. The XML file is 91,980 bytes, created on Oct. 10, 2024, and is being submitted electronically via USPTO Patent Center.

### FIELD

The invention relates generally to populations of Natural Killer (NK) cells and methods of use thereof.

### BACKGROUND

Cancer immunotherapy is utilized for generating and augmenting an anti-tumor immune response, e.g., by treatment with antibodies specific to antigens on tumor cells, with fusions of antigen presenting cells with tumor cells, or by specific activation of anti-tumor NK cells or T cells. The ability of recruiting immune cells against tumor cells in a patient provides a therapeutic modality of fighting cancer types and metastasis that so far were considered incurable.

Lymphocytes such as natural killer (NK) cells are potent anti-tumor effectors that play an important role in innate and adaptive immunity. There are several activating receptors found on NK cells, including NKp30, NKp44, and NKp46, which are collectively known as Natural Cytotoxicity Receptors (NCRs), as well as NKG2D, CD16 and TRAIL. NKp46 is an established marker for the identification of NK cells. NKp46 is an NK cell specific triggering molecule found on both resting and activated NK cells. It is an important mediator in NK cell activation against numerous targets, including tumors and virally infected cells. NK cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells constitute about 10% of the lymphocytes in human peripheral blood. NK cells are effector cells known as large granular lymphocytes because of their larger size and the presence of characteristic azurophilic granules in their cytoplasm. NK cells differentiate and mature in the bone marrow, lymph nodes, spleen, tonsils, and thymus. NK cells can be detected by specific surface markers, such as CD56 and CD45 in humans. NK cells do not express T cell antigen receptors, the pan T marker CD3, or surface immunoglobulin B cell receptors.

Stimulation of NK cells may be achieved through a cross-talk of signals derived from cell surface activating and inhibitory receptors. The activation status of NK cells is regulated by a balance of intracellular signals received from an array of germ-line-encoded activating and inhibitory receptors (MacFarlane and Campbell, Curr Top Microbiol Immunol. 2006; 298:23-57). When NK cells encounter an abnormal cell (e.g., tumor or virus-infected cell) and activating signals predominate, the NK cells can rapidly induce apoptosis of the target cell through directed secretion of cytolytic granules containing perforin and granzymes or engagement of death domain-containing receptors. Activated NK cells can also secrete type I cytokines, such as interferon-γ, tumor necrosis factor-a and granulocyte-macrophage colony-stimulating factor (GM-CSF), which activate both innate and adaptive immune cells as well as other cytokines and chemokines (Wu and Lanier, Adv Cancer Res. 2003; 90:127-56). Production of these soluble factors by NK cells in early innate immune responses significantly influences the recruitment and function of other hematopoietic cells. Also, through physical contacts and production of cytokines, NK cells are central players in a regulatory crosstalk network with dendritic cells and neutrophils to promote or restrain immune responses.

NK cells have several characteristics which make them advantageous to use in therapeutic settings. For example, they do not require antigen priming and they are HLA agnostic, which decreases the risk of developing of Graft vs. Host Disease in allogeneic use. Nonetheless, the use of immune cells for adoptive cell therapies remains challenging and there are unmet needs for improvement. There are significant opportunities that remain to harness the full potential of NK cells, or other lymphocytes in adoptive immunotherapy.

### SUMMARY

In one aspect, provided herein is a cell population comprising differentiated cells derived from induced pluripotent stem cells, said differentiated cells having a Natural Killer cell phenotype (iNK cells), wherein the iNK cells comprise an inactivating mutation in the TGFbR2 gene, and wherein the iNK cells express interleukin-15 (IL-15) or a functional fragment thereof. In some embodiments, the iNK cells comprise a homozygous inactivating mutation in the TGFbR2 gene. In some embodiments, the i cells comprise a heterozygous inactivating mutation in the TGFbR2 gene. In some embodiments, the iPSC-NK cells express a cell membrane-bound form of IL-15 (mbIL-15) or functional fragment thereof. In some embodiments, the iNK cells express a cell membrane-bound form of IL-15 (mbIL-15) or functional fragment thereof fused to the IL-15 Receptor alpha (IL-15Rα). The cell population of any one of claims 1-3, wherein the iNK cells express IL-15 trapped in the endoplasmic reticulum (ER). In some embodiments, the iPSC-NK cells express a soluble form of IL-15 or functional fragment thereof.

In some embodiments, the iPSC-NK cells comprise a knock-in of a polynucleotide encoding the IL-15 or functional fragment thereof into the B2M gene of the iPSC-NK cells. In some embodiments, the iPSC-NK cells comprise a knock-in of a polynucleotide encoding the IL-15 or functional fragment thereof into the TGFbR2 gene of the iPSC-NK cells. In some embodiments, the polynucleotide encoding the IL-15 or functional fragment thereof is operably linked to a promoter. In some embodiments, the promoter is an exogenous promoter. In some embodiments, the promoter is a constitute promoter. In some embodiments, the promoter is an EF1α promoter (short version) or an EF1α promoter (long version). In some embodiments, the promoter is an endogenous promoter.

In some embodiments, the expression level of TGFbR2 in the iNK cells is about 10%, about 20%, about 30%, about 40%, about 50%, about 60%, about 70%, about 80%, about 90%, or about 95% lower than the expression level of TGFbR2 in a population of unedited NK cells.

In some embodiments, at least 50%, at least 55%, at least 60%, at least 65%, or at last 70%, of cells express one or more NK cell marker selected from the group consisting of CD56 and CD45. In another aspect, provided herein is a method of producing a cell population comprising differentiated cells derived from induced pluripotent stem cells, said differentiated cells having a Natural Killer cell phenotype (iPSC-NK cells), wherein the iPSC-NK cells comprise a homozygous inactivating mutation in the TGFbR2 gene, and wherein the iPSC-NK cells express IL-15 or a functional fragment thereof, the method comprising (i) genetically editing a population of induced pluripotent stem cells (iPSCs); (ii) generating a monoclonal population of edited iPSCs; and (iii) differentiating said population of iPSCs into a population of Natural Killer (NK) cells. In some embodiments, step (i) comprises introducing a polynucleotide encoding a soluble form of interleukin-15 (IL-15) or a functional fragment thereof. In some embodiments, step (i) comprises introducing a polynucleotide encoding a cell membrane-bound form of IL-15 (mbIL-15) or functional fragment thereof. In some embodiments, step (i) comprises introducing a polynucleotide encoding mbIL-15 or functional fragment thereof fused to IL-15Rα.

In some embodiments, the IL-15 is introduced using a TALEN construct. In some embodiments, the IL-15 is introduced using a Cas9 or Cas12 enzyme. In some embodiments, the IL-15 or functional fragment thereof is introduced using a dualase platform.

In some embodiments, the methods results in a cell population wherein the expression level of TGFbR2 in the iNK cells is about 10%, about 20%, about 30%, about 40%, about 50%, about 60%, about 70%, about 80%, about 90%, or about 95% lower than the expression level of TGFbR2 in a population of unedited NK cells.

In some embodiments, at least 50%, at least 55%, at least 60%, at least 65%, at last 70%, at least 75%, at least 80%, at least 85%, at least 90%, at least 95%, at least 98% or more than 98% of cells express one or more NK cell marker selected from the group consisting of CD56 and CD45.

In another aspect, provided herein is a cell population produced by a method described herein. In another aspect, provided herein is a pharmaceutical composition comprising a cell population described herein.

In another aspect, provided herein is a method of treating cancer in a subject in need thereof, comprising administering an effective amount of a pharmaceutical composition described herein. In some embodiments, the cancer is a solid tumor. In some embodiments, the cancer is a hematological malignancy

In another aspect, provided herein is a method of inhibiting proliferation of tumor cells in a subject in need thereof, comprising administering an effective amount of a pharmaceutical composition provided herein. In some embodiments, the subject is further administered an immune checkpoint inhibitor. In some embodiments, the immune checkpoint inhibitor is an anti-PD-1 antibody, an anti-PDL-1 antibody, or an anti-CTLA-4 antibody. In some embodiments, the subject is further administered an NK cell engager. In some embodiments, the NK cell engager is an NKp46 NK cell engager. In some embodiments, the subject is further administered an antibody.

### BRIEF DESCRIPTION OF DRAWINGS

FIG. 1 shows an exemplary protocol for manufacturing iPSC-NK cells such as described herein.

FIGS. 2A and 2B show gene editing strategies. FIG. 2A shows a gene structure of human B2M (beta-2-macroglobulin) and non-destructive knock-in (KI) strategy. FIG. 2B shows a gene structure of human TGFβR2 and EF1α-IL15-bGHpA disruptive KI strategy.

FIG. 3 shows donor template designs for the non-destructive KI of IL-15 into the B2M locus (top) and for the disruptive KI of EF1α-IL-15-polyA into the TGFbR2 locus.

FIGS. 4A and 4B show illustrative TALEN target sequences. FIG. 4A shows an illustrative TALEN target sequence for the non-destructive KI of IL-15 into the B2M locus. The B2M TALEN left target sequence is: 5′ TTAGCTGTGCTCGCGCT 3′. The B2M TALEN right target sequence is: 5′ TGGATAGCCTCCAGGCC 3′. FIG. 4B shows an illustrative TALEN target sequence for the disruptive KI of EF1α-IL-15-polyA into the TGFbR2 locus. The TGFbR2 TALEN left target sequence is: 5′ TGATGTGAGATTTTCCA 3′. The TGFbR2 TALEN right target sequence is: 5′ TTGCTCATGCAGGATTT 3′.

FIG. 5 shows sequencing results of the IL-15-P2A-B2M joint area of the biallelic IL-15 knock-in iPSC single colonies.

FIG. 6 shows sequencing results of the TGFbR2 locus in TGFbR2 knockout (KO) iPSC cells.

FIGS. 7A and 7B show amplicon sequencing results for the iPSC clone #269 which is a true TGFbR2 KO clone. FIG. 7A is a graph to show the reading % on TGFbR2 locus.

FIG. 7B is a detailed analysis of those readings.

FIGS. 8A and 8B show amplicon sequencing results for the iPSC clone #331 which is a true TGFbR2 KO clone together with IL-15 non-destructively KI on B2M locus. FIG. 8A is a graph to show the reading % on TGFbR2 locus. FIG. 8B is a detailed analysis of those readings.

FIG. 9 shows sequencing results of clone #337, with an IL-15 Ki in the B2M locus and a TGFbR2 deletion.

FIG. 10 shows sequencing results of clone #338, with an IL-15 Ki in the B2M locus and a TGFβR2 deletion.

FIG. 11 shows Sanger sequencing results for clones #336, and 341.

FIG. 12 shows IL-15 levels in culture medium from iPSC clones #231, #238, #239, and #242 as determined by ELISA.

FIG. 13 shows TGFbR2 protein in clones #255, #260, and #269 as detected by Western Blot.

FIG. 14 shows TGFbR2 protein in iPSC clones #331, #336, #337, #338, #341, and #318 as detected by Western Blot.

FIG. 15 shows TGFbR2 expression in iNK cell differentiated from iPSC clone #269.

FIG. 16 shows IL-15 expression in the medium of iNK cells differentiated from iPSC clones #242, #318, #336, and #341 as determined by ELISA.

FIG. 17 shows the impact of TGFb1 on cell surface expression of NKG2D, DNAM-1, NKp30 and CD2.

FIG. 18 shows the impact of IL-15 on in vitro cell persistence.

FIG. 19 shows the impact of TGFb1 on cytotoxic activity of iNK cells differentiated from iPSC clones #269, #318, and #341.

FIGS. 20A and 20B show cell killing activities of iNK cells differentiated from iPSC clones #231, #242, #269, #318, #336, and #341 after two and three rounds, respectively, of challenge with target cells.

### DETAILED DESCRIPTION

The present disclosure provides modified Natural Killer (NK) cells for use in the treatment of cancer. In some embodiments, the NK cells may have one or more genetic modifications, for example, knock-out of TGF beta receptor 2 (TGFbR2) and/or knock-in of interleukin 15 (IL-15).

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

As used in the specification and claims, the singular form “a”, “an” and “the” include plural references unless the context clearly dictates otherwise. For example, the term “a cell” includes a plurality of cells, including mixtures thereof.

### NK Cells

In one aspect, provided herein is a cell population comprising differentiated cells derived from induced pluripotent stem cells, said differentiated cells having a Natural Killer cell phenotype. These NK cells may be derived from iPSCs are also referred to interchangeably as “iPSC-NK cells” or “INK cells.”

Methods of Making iPSC-NK Cells

Any suitable source of iPSC may be used to generate the iPSC-NK cells provided herein. Thus, in one embodiment, provided herein is a method of producing a cell population comprising iPSC-NK cells, wherein the iPSC-NK cells comprise a homozygous inactivating mutation in the TGFbR2 gene, and wherein the iPSC-NK cells express IL-15 or a functional fragment thereof, the method comprising (i) genetically editing a population of induced pluripotent stem cells (iPSCs); (ii) differentiating said population of iPSCs into a population of Natural Killer (NK) cells; and (iii) expanding the population of NK cells.

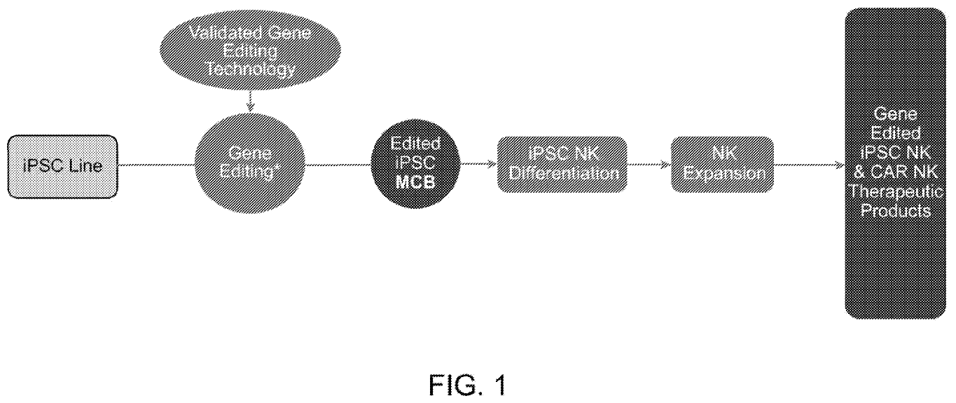

An exemplary method is illustrated in FIG. 1. For example, iPSC may be produced by reprogramming somatic cells to induce pluripotency. The reprogramming involves the activation of pluripotency genes and repression of somatic genes. In its simplest form, this process includes the expression of certain transcription factors in the somatic cells, in particular Octamer 3/4 (Oct3/4), SRY-box containing gene 2 (Sox2), Krüppel-like factor 4 (Klf4), and the protooncogene cytoplasmic Myc protein (c-Myc) (see Takahashi and Yamanaka, Cell 126:663-676). The delivery of these transcription factors into the somatic cells can be accomplished by any suitable method known in the art, for example, using viral vectors, mRNA transfection, or delivery of recombinant proteins (see, e.g. Chang et al., 2019, J Korean Neurosurg Soc. 62 (5): 493-501). Alternatively or additionally, small molecules targeting cell signaling pathways, metabolic pathways, and epigenetic modifications may be used to induce pluripotency and reprogram somatic cells in to iPSCs. In particular, repression of DNA methylation, activation of Wnt signaling, activation of MAPK/ERK signaling, and induction of glycolytic metabolism have been described as mechanisms to aid in reprogramming. Such small molecules include, for example, Gsk3β inhibitors, transforming growth factor β (TGFβ) inhibitors, TGFR inhibitors, MEK inhibitors, AMPK inhibitors, mTOR inhibitors, VEGF inhibitors, Wnt activators, cAMP activators, retinoic acid receptor (RAR) α agonists, RAR γ agonists, pyruvate dehydrogenase kinase, isozyme 1 (PDK-1) activators, HMT inhibitors, DNMT inhibitors, KDM inhibitors, HDAC inhibitors, and others. See, e.g., Liu et al., 2019, Cell Chem Biol; 23:893-916, which is incorporated herein in its entirety for examples of small molecules that may be used in the methods of generating iPSC.

Commercial iPSC lines are also available and may be used to generate the iPSC-NK cells described herein.

In preferred embodiments, the iPSCs used to generate the iPSC-NK cells provided herein are generated, maintained and differentiated under Good Manufacturing Protocol (GMP) conditions.

iPSCs may be differentiated into NK cells using any suitable method known in the art or described herein. A description of such methods is described in, for example, Zhu, H., Kaufman, D. S. (2019). An Improved Method to Produce Clinical-Scale Natural Killer Cells from Human Pluripotent Stem Cells. In: Kaneko. S. (eds) In Vitro Differentiation of T-Cells. Methods in Molecular Biology, vol 2048. Humana, New York, NY; https://doi.org/10.1007/978-1-4939-9728-2_12, which is incorporated herein by reference in its entirety. For example, the iNK cells may be activated for three days at high concentrations of IL-2 (100 unit/ml to 500 unit/ml), and with additional cytokines IL-15 and IL-21A.

The iPSC-NK cells provided herein may be cultured under any suitable conditions described herein or known in the art. In some embodiments, the NK cells are cultured on a feeder layer, i.e., in co-culture with another cell line. Such co-cultures can be effective in inducing proliferation in cell types that otherwise proliferate very slowly or not at all. In some embodiments, provided herein is a feeder layer that is capable of inducing proliferation of iPSC-NK cells. In some embodiments, provided herein is a feeder layer that is capable of activating iPSC-NK cells.

Examples of feeder layers that may be used for the culture of NK cells provided herein include, without limitation, K562 cells and 221 cells. In some embodiments, the feeder layer cells may be genetically modified, e.g., the feeder layer cells may be transduced with mbIL-15, mIL21 and/or 4-1-BB. Prior to being used in the co-culture, the feeder layer cells may be irradiated with doses sufficient to induce cell cycle arrest, such that the feeder layer cells do not proliferate in the co-culture.

In some embodiments, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90 to about 95%, or about 95% to 100% of cells in a population of iPSC-NK cells provided herein express one or more NK cell marker such as CD56 and/or CD45.

In some embodiments, at least 50%, at least 55%, at least 60%, at least 65%, at last 70%, at least 75%, at least 80%, at least 85%, at least 90%, at least 95%, at least 98% or more than 98% of cells in a population of iPSC-NK cells provided herein express one or more NK cell marker such as CD56 and/or CD45. NK cell surface markers such as CD56 and CD45 may be detected using any suitable method known in the art, including, for example, flow cytometry.

### Modification of NK Cells

In some embodiments, the iPSC-NK cells provided herein cells are genetically modified by introducing (“integrating” or “knocking in”) or deleting (“knocking out”) one or more genes. Without wishing to be bound by theory, knocking out or integrating genes of interest involved in NK cell exhaustion, activation, tolerance, and/or memory are thought to improve the clinical utility of the iPSC-NKs provided herein.

The genetic modification of the iPSC-NK cells provided herein may be achieved by any suitable method known in the art or described herein. For example, the genome of the iPSC-NK cells provided herein may be modified by introducing DNA double strand breaks, which are then repaired by the cell's endogenous repair mechanisms, such as homologous recombination. DNA double strand breaks may be introduced using targeted endonucleases, such as Zinc-finger nucleases, transcription activation-like effector-nucleases (TALENs), meganucleases, or the CRISPR/Cas system, which relies on the Cas9 endonuclease for inducing the DNA breaks and a guide RNA (gRNA) for site-specificity.

Alternatively, a Dualase™ platform may be used to edit the iPSC cells described herein. The Dualase™ is a gene editing technology which cuts DNA twice and leaves non-compatible DNA ends, which is hypothesized to lead to higher fidelity repair than non-compatible ends.

TALENs employ a bacterial DNA cleavage domain and specifically bind DNA via highly conserved 33-35 amino acid TALE repeats which resemble the DNA-binding domains of transcription factors. The TALE repeats each bind a single base pair of DNA. The specificity of TALEN DNA binding is dictated by two hypervariable residues. The repeats for a two-helix structure connected by a loop which presents the hypervariable residue into the major groove of the DNA. Multiple modular TALE repeats can be linked together into a longer array with custom DNA-binding specificities. See e.g., Maeder and Gersbach, 2016, Mol Ther. 24 (3) 430-446; Carrol, 2017, Yale J Biol Med 90:653-659.

Methods for designing TALEN sequences targeting a desired locus are well known in the art and described in, e.g., Cermak et al., Nucleic Acids Res. 2011 Jul; 39 (12): e82, which is incorporated herein in its entirety.

Cas-based DNA editing systems are well known in the art. Any suitable Cas enzyme can be used to edit the iPSC-NK cells described herein, including, without limitation, Cas9 and Cas12.

In the cases of knock-in, the polynucleotide encoding the knocked-in gene is introduced in such a way that the polynucleotide is operatively linked to a promoter. The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences which are necessary to affect the expression and processing of coding sequences to which they are ligated. The nature of such control sequences differs depending upon the host organism in prokaryotes, such control sequences generally include promoter, ribosomal binding site, and transcription termination sequence in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences. The term “polynucleotide” as referred to herein means a polymeric boron of nucleotides of at least 10 bases in length, either ribonucleotides or deoxynucleotides or a modified form of either type of nucleotide. The term includes single and double stranded forms of DNA.

In some embodiments, the iPSC-NK cells are modified by knocking in a gene encoding interleukin 15 (IL-15) or a functional fragment thereof. By “functional fragment” is meant a fragment of a protein (e.g., IL-15) which retains one or more desired activities of the parental protein. In some embodiments, the iPSC-NK cells provided herein are modified by introducing an IL15 gene. Thus, provided herein are iPSC-NK cells which express IL-15 or a functional fragment thereof.

Several isoforms of IL-15 are known in the art and may be used in the iPSC-NK cells provided herein. Exemplary sequences of IL-15 isoforms are provided in Table 1. In some embodiments, an iPSC-NK cells provided herein expresses a polypeptide comprising the sequence of SEQ ID NO: 1. In some embodiments, an iPSC-NK cells provided herein expresses a polypeptide comprising a sequence that is at least 70%, at least 75%, at least 80%, at least 85%, at least 90%, at least 95%, or at least 98% identical to the sequence of SEQ ID NO: 1. In some embodiments, an iPSC-NK cells provided herein expresses a polypeptide comprising the sequence of SEQ ID NO: 2. In some embodiments, an iPSC-NK cells provided herein expresses a polypeptide comprising a sequence that is at least 70%, at least 75%, at least 80%, at least 85%, at least 90%, at least 95%, or at least 98% identical to the sequence of the sequence of SEQ ID NO: 2.

In some embodiments, an iPSC-NK cells provided herein comprises a polynucleotide encoding the amino acid sequences of SEQ ID NO: 1. In some embodiments, an iPSC-NK cells provided herein comprises a polynucleotide encoding a sequence that is at least 70%, at least 75%, at least 80%, at least 85%, at least 90%, at least 95%, or at least 98% identical to the amino acid sequences of SEQ ID NO: 1. In some embodiments, an iPSC-NK cells provided herein comprises a polynucleotide encoding the amino acid sequences of SEQ ID NO: 2. In some embodiments, an iPSC-NK cells provided herein comprises a polynucleotide encoding a sequence that is at least 70%, at least 75%, at least 80%, at least 85%, at least 90%, at least 95%, or at least 98% identical to the amino acid sequences of SEQ ID NO: 2.

TABLE 1
Exemplary IL-15 Sequences

| Name | Sequence |
|---|---|
| Human IL-15 | MRISKPHLRSISIQCYLCLLLNSHFLTEAGIHVF |
| isoform | ILGCFSAGLPKTEANWVNVISDLKKIEDLIQSMH |
| S48AA | IDATLYTESDVHPSCKVTAMKCFLLELQVISLES |
| (UniProt | GDASIHDTVENLIILANNSLSSNGNVTESGCKEC |
| Accession | SEELEEKNIKEFLQSFVHIVQMFINT (SEQ ID |
| No. P40933-1) | NO: 1) |
| Human IL-15 | MVLGTIDLCSCFSAGLPKTEANWVNVISDLKKIE |
| isoform | DLIQSMHIDATLYTESDVHPSCKVTAMKCFLLEL |
| S21AA | QVISLESGDASIHDTVENLIILANNSLSSNGNVT |
| (UniProt | ESGCKECEELEEKNIKEFLQSFVHIVQMFINTS |
| Accession | (SEQ ID NO: 2) |
| No. P40933-1) | |

The IL-15 expressed by the iPSC-NK cells provided herein may be a soluble form of IL15. An illustrative nucleic acid sequence encoding a soluble form of IL-15 is set forth in SEQ ID NO: 41.

The IL-15 or functional fragment thereof may be a membrane-bound form of IL-15 (mbIL-15) or a functional fragment thereof. Expression of a membrane-bound form of IL-15 may be achieved by expressing a nucleic acid encoding a soluble form of IL-15 (e.g., SEQ ID NO: 41) as a fusion protein with the hinge and transmembrane domains of CD8. An illustrative nucleic acid sequence encoding the hinge and transmembrane domains of CD8 is set forth in SEQ ID NO: 46.

The IL-15 or functional fragment thereof may be a form of IL-15 that is trapped in the endoplasmic reticulum (ER). This may be achieved by, for example, expressing a polynucleotide encoding IL-15 or a functional fragment thereof (e.g., SEQ ID NO: 41) as a fusion protein with an ER retention signal. An illustrative ER retention signal sequence is GSEKDEL (SEQ ID NO: 54) and an illustrative nucleic acid sequence encoding an ER retention signal is set forth in SEQ ID NO: 50.

The IL-15 or functional fragment thereof may be a form of IL-15 (e.g., a membrane-bound form of IL-15, a soluble form of IL-15, or an ER-trapped form of IL-15) or functional fragment thereof fused to the IL-15 receptor alpha (IL-15Rα). This may be accomplished by expressing the IL-15 or functional fragment thereof as a fusion protein with IL-15Rα. An illustrative nucleic acid sequence encoding IL-15Rα is set forth in SEQ ID NO: 43.

In another aspect, provided herein are polypeptides and vectors comprising the gRNAs and/or the donor DNA sequences provided herein. The gRNAs provided herein may be used in combination with any suitable DNA editing enzyme known in the art or described herein, including, for example, Cas9 and Cas12.

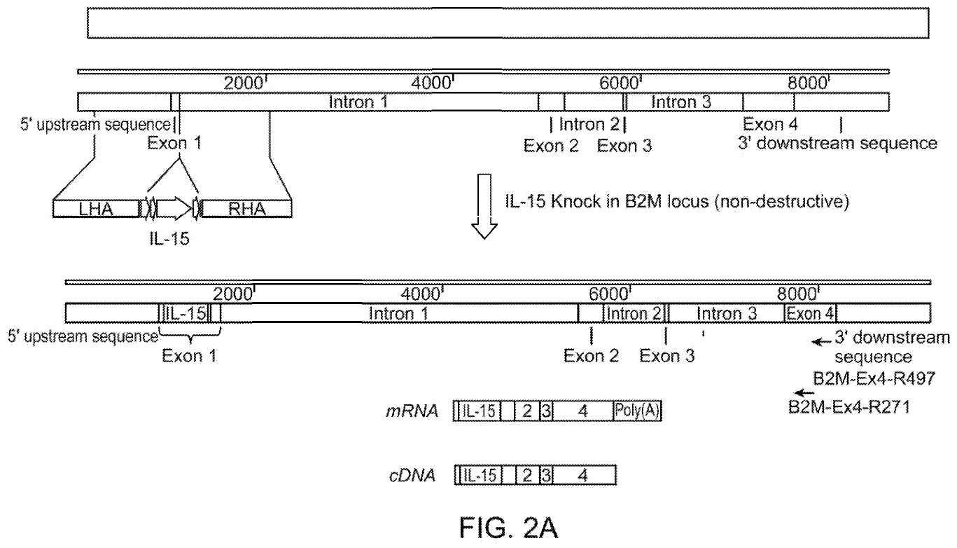

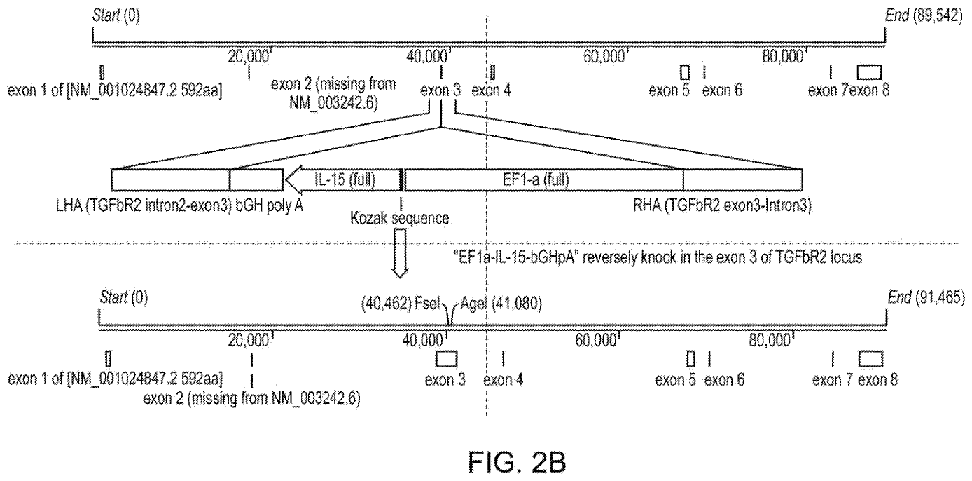

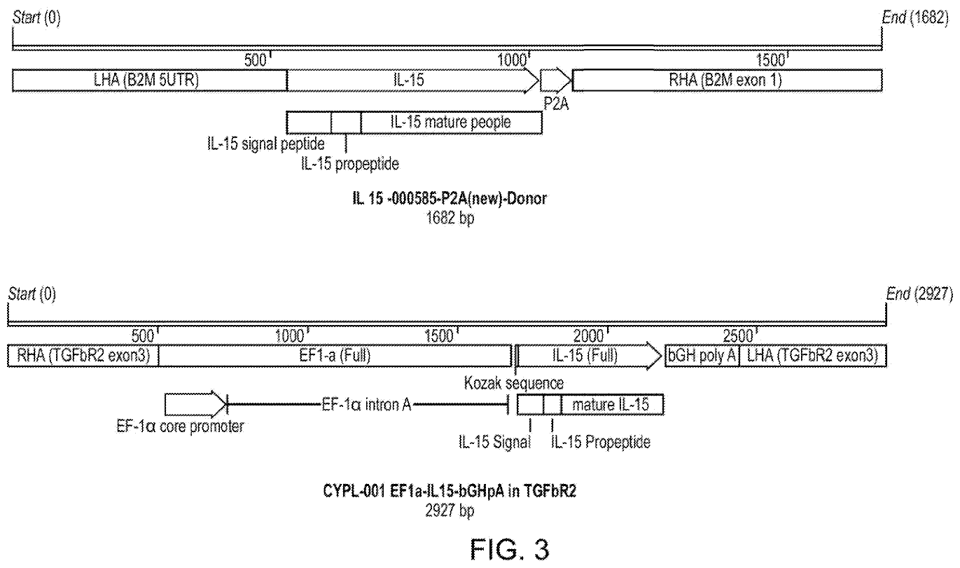

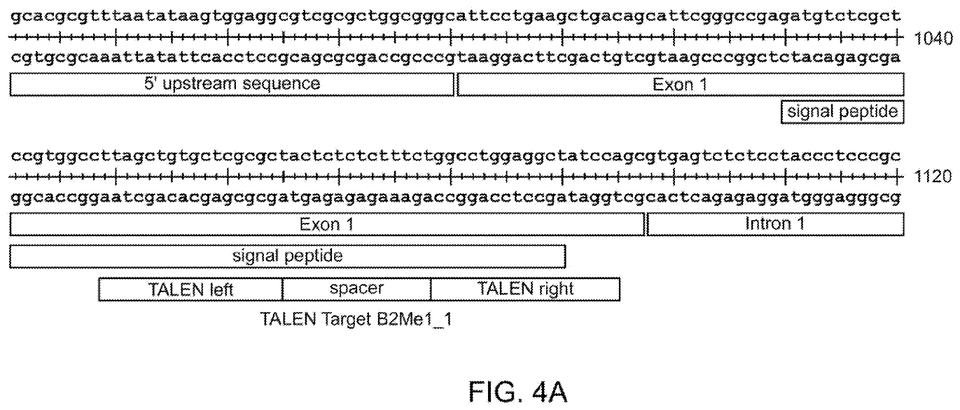

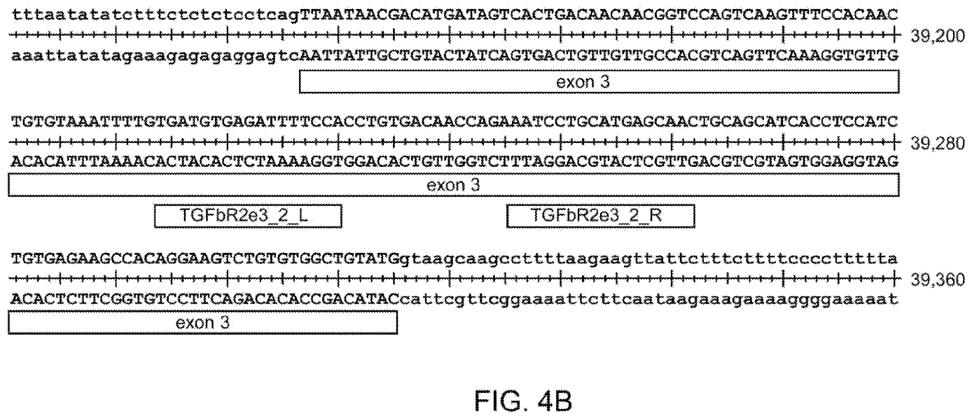

The IL-15 gene may be knocked into the iPSC-NK cell genome at any suitable position. In certain embodiments the integration locus is the B2M locus. In certain embodiments, the integration locus is the CD38 locus. In certain embodiments, the integration locus is the TGFbR2 locus (Gene ID: 7048). An illustrative gene editing strategy for a non-disruptive knock-in of IL-15 into the B2M locus is shown in FIG. 2A. An illustrative gene editing strategy for a disruptive knock-in of IL-15 into the TGFbR2 locus is shown in FIG. 2B.

Exemplary gRNAs that may be used for the knock-in of IL-15 at the B2M locus are shown in Table 2. Nucleotides 1-20 of each of SEQ ID NOs: 3 and 4 are the gRNA sequence and nucleotides 20-23 of each of SEQ ID NOs: 3 and 4 are the protospacer adjacent motif (PAM).

In some embodiments, provided herein is a nucleic acid donor construct that may be used to deliver a nucleic acid sequence encoding IL-15 or a functional fragment thereof to a target site in the genome. Illustrative donor sequences that may be used to insert IL-15 or a functional fragment thereof into a cell are set forth in SEQ ID NOs: 27-38.

Generally, a construct that may be used to deliver a polynucleotide encoding IL-15 or a functional fragment thereof to a target site (e.g., a B2M target site or a TGFbR2 target site) comprises a nucleic acid sequence encoding IL-15 or a functional fragment thereof, flanked by a left homology arm (LHA) and a right homology arm (RHA). The LHA and RHA of a given donor construct comprise nucleic acid sequences with homology to the target site (e.g., the B2M locus or the TGFbR2 locus).

The sequence and length of the RHA and LHA sequences may vary based on the targeted site. In some embodiments, the LHA sequence comprises a nucleic acid sequence that is homologous to the 5′ upstream sequence of the B2M gene. In some embodiments, the RHA sequence comprises a nucleic acid sequence that is homologous to exon 1 and intron 1 of B2M. An illustrative LHA-RHA sequence pair that may be used for targeted insertion into the B2M locus is the pair of sequences set forth in SEQ ID NO: 39 (LHA) and SEQ ID NO: 45 (RHA).

In some embodiments, the LHA sequence comprises a nucleic acid sequence that is homologous to intron 2 and exon 3 of TGFbR2. In some embodiments, the RHA sequence comprises a nucleic acid sequence that is homologous to exon3 and intron 3 of TGFbR2. An illustrative LHA-RHA sequence pair that may be used for targeted insertion into the TGFbR2 locus is the pair of sequences set forth in SEQ ID NO: 52 (LHA) and SEQ ID NO: 47 (RHA).

In some embodiments, the donor construct further comprises one or more spacer domains, one or more insulator domains, a CD8-hinge-transmembrane domain, a promoter, an endoplasmic reticulum (ER) retention signal sequence, a polyA sequence (e.g., a bGHpA sequence), and/or an IRES element (e.g., an IRES2 element).

The elements of a nucleic acid construct may be separated by spacer elements, insulators, and/or 2A sequences (e.g., a P2A sequence) . . .

TABLE 2
Exemplary gRNA Sequences for IL-15 Knock-in at the
B2M Locus.

| Name | Sequence |
|---|---|
| B2M gRNA #1 | GGCCGAGATGTCTCGCTCCGTGG |
| | (SEQ ID NO: 3) |
| B2M gRNA #2 | GGCCACGGAGCGAGACATCTCGG |
| | (SEQ ID NO: 4) |

An exemplary donor DNA sequence that may be used for the knock-in of IL-15 at the B2M locus using CRISPR is set forth in SEQ ID NO: 5. Nucleotides 5-811 of SEQ ID NO: 5 are the left homology arm (LHA), nucleotides 955-1296 of SEQ ID NO: 5 are encode IL-15, and nucleotides 1363-2205 of SEQ ID NO: 5 are the right homology arm (RHA).

```
(SEQ ID NO: 5)
      1 AAACTAAGAG AATGATGTAC CTAGAGGGCG CTGGAAGCTC TAAAGCCCTA
  51 GCAGTTACTG CTTTTACTAT TAGTGGTCGT TTTTTTCTCC CCCCCGCCCC
  101 CCGACAAATC AACAGAACAA AGAAAATTAC CTAAACAGCA AGGACATAGG
  151 GAGGAACTTC TTGGCACAGA ACTTTCCAAA CACTTTTTCC TGAAGGGATA
  201 CAAGAAGCAA GAAAGGTACT CTTTCACTAG GACCTTCTCT GAGCTGTCCT
  251 CAGGATGCTT TTGGGACTAT TTTTCTTACC CAGAGAATGG AGAAACCCTG
  301 CAGGGAATTC CCAAGCTGTA GTTATAAACA GAAGTTCTCC TTCTGCTAGG
  351 TAGCATTCAA AGATCTTAAT CTTCTGGGTT TCCGTTTTCT CGAATGAAAA
  401 ATGCAGGTCC GAGCAGTTAA CTGGCGGGGG CACCATTAGC AAGTCACTTA
  451 GCATCTCTGG GGCCAGTCTG CAAAGCGAGG GGGCAGCCTT AATGTGCCTC
  501 CAGCCTGAAG TCCTAGAATG AGCGCCCGGT GTCCCAAGCT GGGGCGCGCA
  551 CCCCAGATCG GAGGGCGCCG ATGTACAGAC AGCAAACTCA CCCAGTCTAG
  601 TGCATGCCTT CTTAAACATC ACGAGACTCT AAGAAAAGGA AACTGAAAAC
  651 GGGAAAGTCC CTCTCTCTAA CCTGGCACTG CGTCGCTGGC TTGGAGACAG
  701 GTGACGGTCC CTGCGGGCCT TGTCCTGATT GGCTGGGCAC GCGTTTAATA
  751 TAAGTGGAGG CGTCGCGCTG GCGGGCATTC CTGAAGCTGA CAGCATTCGG
  801 GCCGAGATGC GCATAAGTAA GCCTCACTTG AGATCTATTA GCATACAGTG
  851 TTACCTGTGC CTTCTCCTCA ATTCTCACTT CCTGACAGAA GCCGGAATCC
  901 ACGTTTTTAT TCTCGGTTGC TTCAGTGCCG GACTGCCTAA GACGGAAGCT
  951 AATTGGGTCA ACGTTATAAG CGATCTCAAA AAAATCGAAG ATTTGATACA
1001 GAGCATGCAC ATTGATGCTA CTTTGTATAC AGAAAGTGAC GTACACCCAA
1051 GTTGTAAAGT AACGGCGATG AAATGTTTCC TGCTCGAGCT GCAGGTAATC
1101 AGCCTCGAGT CTGGTGACGC CTCAATACAT GACACCGTTG AGAATTTGAT
1151 AATCCTTGCG AATAACTCCT TGTCTTCAAA TGGCAACGTT ACGGAAAGCG
1201 GGTGCAAAGA ATGCGAAGAG TTGGAGGAGA AAAACATTAA GGAATTCCTT
1251 CAGAGTTTTG TACACATAGT GCAAATGTTC ATTAATACAA GCGGATCTGG
1301 AGCAACAAAC TTCTCACTAC TCAAACAAGC AGGTGACGTG GAGGAGAATC
1351 CCGGCCCCAT GTCTCGCTCC GTGGCCTTAG CTGTGCTCGC GCTACTCTCT
1401 CTTTCTGGCC TGGAGGCTAT CCAGCGTGAG TCTCTCCTAC CCTCCCGCTC
1451 TGGTCCTTCC TCTCCCGCTC TGCACCCTCT GTGGCCCTCG CTGTGCTCTC
1501 TCGCTCCGTG ACTTCCCTTC TCCAAGTTCT CCTTGGTGGC CCGCCGTGGG
1551 GCTAGTCCAG GGCTGGATCT CGGGGAAGCG GCGGGGTGGC CTGGGAGTGG
1601 GGAAGGGGGT GCGCACCCGG GACGCGCGCT ACTTGCCCCT TTCGGCGGGG
1651 AGCAGGGGAG ACCTTTGGCC TACGGCGACG GGAGGGTCGG GACAAAGTTT
1701 AGGGCGTCGA TAAGCGTCAG AGCGCCGAGG TTGGGGGAGG GTTTCTCTTC
1751 CGCTCTTTCG CGGGGCCTCT GGCTCCCCCA GCGCAGCTGG AGTGGGGGAC
1801 GGGTAGGCTC GTCCCAAAGG CGCGGCGCTG AGGTTTGTGA ACGCGTGGAG
1851 GGGCGCTTGG GGTCTGGGGG AGGCGTCGCC CGGGTAAGCC TGTCTGCTGC
1901 GGCTCTGCTT CCCTTAGACT GGAGAGCTGT GGACTTCGTC TAGGCGCCCG
1951 CTAAGTTCGC ATGTCCTAGC ACCTCTGGGT CTATGTGGGG CCACACCGTG
2001 GGGAGGAAAC AGCACGCGAC GTTTGTAGAA TGCTTGGCTG TGATACAAAG
2051 CGGTTTCGAA TAATTAACTT ATTTGTTCCC ATCACATGTC ACTTTTAAAA
2101 AATTATAAGA ACTACCCGTT ATTGACATCT TTCTGTGTGC CAAGGACTTT
2151 ATGTGCTTTG CGTCATTTAA TTTTGAAAAC AGTTATCTTC CGCCATAGAT
2201 A
```

An illustrative donor sequence for inserting a membrane-bound form of IL-15 fused to IL-15Rα into the B2M locus of a cell is shown in SEQ ID NO: 27:

LHA(B2M)-mbIL15-IL15Ra-P2A-RHA(B2M)
1. LHA(B2M 5′upstream): SEQ ID NO: 39
2. CD8a(SP): SEQ ID NO: 40

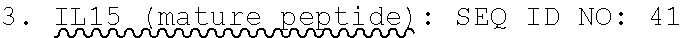

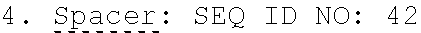

5. IL15RA (mature peptide): SEQ ID NO: 43

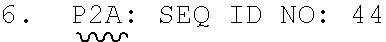

7. RHA (B2M exon1-intron1): SEQ ID NO: 45
(SEQ ID NO: 27)
gcaagaaaggtactctttcactaggaccttctctgagctgtcctcaggatgcttttgggactatttttcttacccagagaatggagaaaccc
tgcagggaattcccaagctgtagttataaacagaagttctccttctgctaggtagcattcaaagatcttaatcttctgggtttccgttttctcg
aatgaaaaatgcaggtccgagcagttaactggctggggcaccattagcaagtcacttagcatctctggggccagtctgcaaagcgag
ggggcagccttaatgtgcctccagcctgaagtcctagaatgagcgcccggtgtcccaagctggggcgcgcaccccagatcggagg
gcgccgatgtacagacagcaaactcacccagtctagtgcatgccttcttaaacatcacgagactctaagaaaaggaaactgaaaacgg
gaaagtccctctctctaacctggcactgcgtcgctggcttggagacaggtgacggtccctgcgggccttgtcctgattggctgggcac
gcgtttaatataagtggaggcgtcgcgctggcgggcATTCCTGAAGCTGACAGCATTCGGGCCGAGAT
GGCCTTACCAGTGACCGCCTTGCTCCTGCCGCTGGCCTTGCTGCTCCACGCCGCC

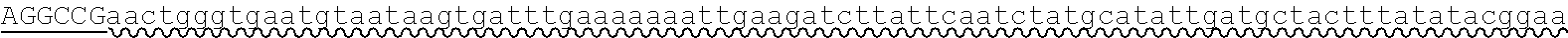

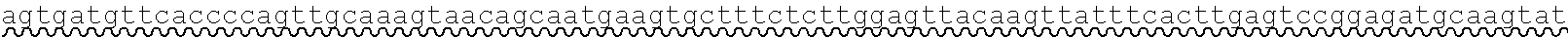

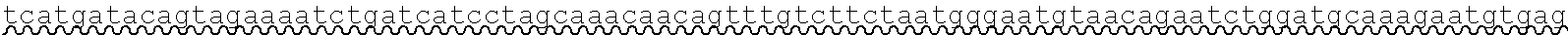

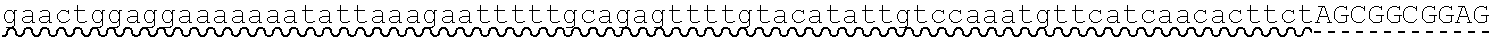

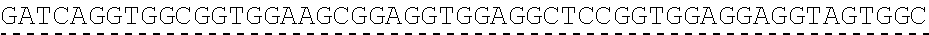

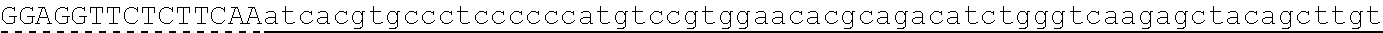

actccagggagcggtacatttgtaactctggtttcaagcgtaaagccggcacgtccagcctgacggagtgcgtgttgaacaaggccac
gaatgtcgcccactggacaacccccagtctcaaatgcattagagaccctgccctggttcaccaaaggccagcgccaccctccacagt
aacgacggcaggggtgaccccacagccagagagcctctccccttctggaaaagagcccgcagcttcatctcccagctcaaacaaca
cagcggccacaacagcagctattgtcccgggctcccagctgatgccttcaaaatcaccttccacaggaaccacagagataagcagtc
atgagtcctcccacggcaccccctctcagacaacagccaagaactgggaactcacagcatccgcctcccaccagccgccaggtgtg
tatccacagggccacagcgacaccactgtggctatctccacgtccactgtcctgctgtgtgggctgagcgctgtgtctctcctggcatg
ctacctcaagtcaaggcaaactcccccgctggccagcgttgaaatggaagccatggaggctctgccggtgacttgggggaccagca

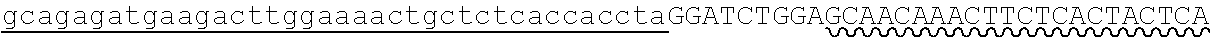

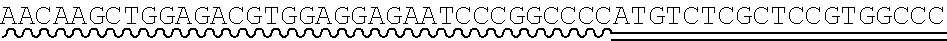

TTGCTGTCCTCGCCTTACTCTCTCTTTCTGGCCTGGAGGCTATCCAGCgtgagtctctccta
ccctcccgctctggtccttcctctcccgctctgcaccctctgtggccctcgctgtgctctctcgctccgtgacttcccttctccaagttctect
tggtggcccgccgtggggctagtccagggctggatctcggggaagcggcggggtggcctoggagtggggaagggggtgcgcac
ccgggacgcgcgctacttgcccctttcggcggggagcaggggagacctttggcctacggcgacgggagggtcgggacaaagttta
gggcgtcgataagcgtcagagcgccgagottgggggagggtttctcttccgctctttcgcggggcctctggctcccccagcocagct
ggagtgggggacgggtaggctcgtcccaaaggcgcggcgctgaggtttotgaacgcgtggaggggcgcttggggtctgggggag
gcgtcgcccgggtaagcctgtctgctgcggctctgcttcccttagactggagagctgtggacttcgtctaggcgcccgctaagttc

An illustrative donor sequence for inserting a membrane-bound form of IL-15 into the B2M locus of a cell is shown in SEQ ID NO: 28:

LHA(B2M)-mbIL15-P2A-RHA(B2M)
1. LHA(B2M 5′upstream): SEQ ID NO: 39
2. CD8A(SP): SEQ ID NO: 40

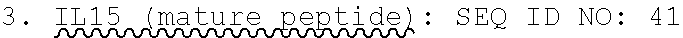

4. CD8a-hinge-TM: SEQ ID NO: 46

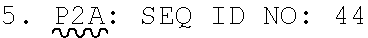

6. RHA (B2M exon1-intron1): SEQ ID NO: 45
(SEQ ID NO: 28)
gcaagaaaggtactctttcactaggaccttctctgagctgtcctcaggatgcttttgggactatttttcttacccagagaatggagaaaccc
tgcagggaattcccaagctgtagttataaacagaagttctccttctgctaggtagcattcaaagatcttaatcttctgggtttccgttttctcg
aatgaaaaatgcaggtccgagcagttaactggctggggcaccattagcaagtcacttagcatctctggggccagtctgcaaagcgag
ggggcagccttaatgtgcctccagcctgaagtcctagaatgagcgcccggtgtcccaagctggggcgcgcaccccagatcggagg
gcgccgatgtacagacagcaaactcacccagtctagtgcatgccttcttaaacatcacgagactctaagaaaaggaaactgaaaacgg
gaaagtccctctctctaacctggcactgcgtcgctggcttggagacaggtgacggtccctgcgggccttgtcctgattggctgggcac
gcgtttaatataagtggaggcgtcgcgctggcgggcATTCCTGAAGCTGACAGCATTCGGGCCGAGAT
GGCCTTACCAGTGACCGCCTTGCTCCTGCCGCTGGCCTTGCTGCTCCACGCCGCC

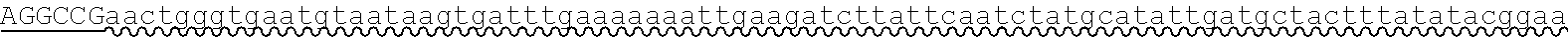

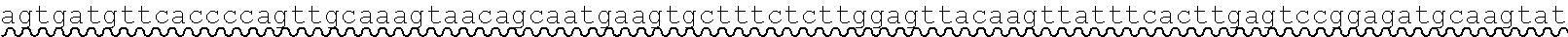

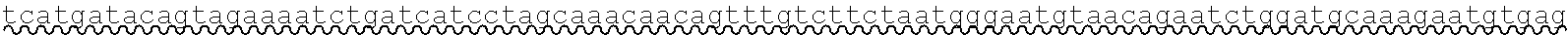

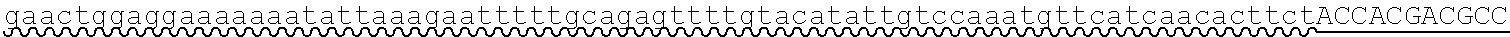

AGCGCCGCGACCACCAACACCGGCGCCCACCATCGCGTCGCAGCCCCTGTCCCT
GCGCCCAGAGGCGTGCCGGCCAGCGGGGGGGGCGCAGTGCACACGAGGGGGC
TGGACTTCGCCTGTGATATCTACATCTGGGCGCCCTTGGCCGGGACTTGTGGGGT

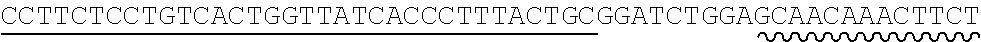

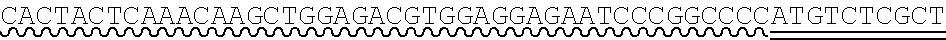

CCGTGGCCCTTGCTGTCCTCGCCTTACTCTCTCTTTCTGGCCTGGAGGCTATCCAG
Cgtgagtctctcctaccctcccgctctggtccttcctctcccgctctgcaccctctgtggccctcgctgtgctctctcgctccgtgacttcc
cttctccaagttctccttggtggcccgccgtggggctagtccagggctggatctcggggaagcggcggggtogcctgggagtgggg
aagggggtgcgcacccgggacgcgcgctacttgcccctttcggggggagcaggggagacctttggcctacggcgacgggaggg
tcgggacaaagtttagggcgtcgataagcgtcagagcgccgaggttgggggagggtttctcttccgctctttcgcggggcctctggct
cccccagcgcagctggagtgggggacgggtaggctcgtcccaaaggcgcggcgctgaggtttgtgaacgcgtggaggggcgctt
ggggtctgggggaggcgtcgcccgggtaagcctgtctgctgcggctctgcttcccttagactggagagctgtggacttcgtctaggcg
cccgctaagttc

An illustrative donor sequence for inserting a form of IL-15 fused to IL-15Rα into the TGFbR2 locus of a cell is shown in SEQ ID NO: 29:

RHA(TGFbR2)-insul-EF1a-IL15-IL15Ra-bGHpA-insul-LHA(TGFbR2)
1. RHA(TGFbR2 exon3-intron3): SEQ ID NO: 47

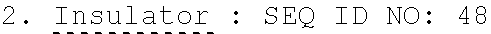

3. EFla (full length): SEQ ID NO: 49
4. CD8a (SP): 40

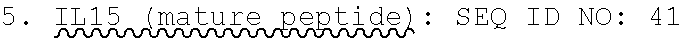

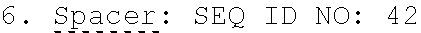

7. IL 15RA (mature peptide): SEQ ID NO: 43
8. bGHpA: 51

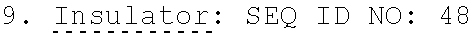

10. LHA (TGFbR2 intron2-exon3): SEQ ID NO: 52
(SEQ ID NO: 29)
tatatgaataatataaccagaatttctgactaaaaaaacaggttgtttttgtggttctccaagtagattccccatttttggctggtagttttctctt
ccaacagccaacctttcatcacctcttcttccagacctgagattgcctcaagagaacaaagctcctggtctcactatggggtgctgagga
ggtgtcgottaaatgactactaagcggcaaatccctcttcttgatacaagaaagagtttccagatttattatattaaagatcattttatgatctt
tacatttatcatgatcattttataatcatgatacgtttatatacccacataaacgtacacatacatgcagagaacacccctagaaactacattt
aataatcgaaagagagatggtctaaaggaaagggaaatggaacaggtgtttacatttaggagacagagatacactgactgtgtgtacta
tgagaatacattatgtaaaaaggggaaaagaaagaataacttcttaaaaggcttgcttacCATACAGCCACACAGACT
TCCTGTGGCTTCTCACAGATGGAGGTGATGCTGCAGTTGCTCATGCAGGATTTCT

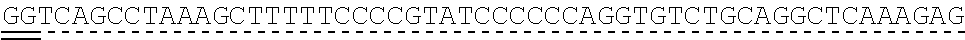

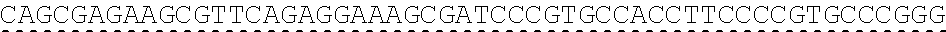

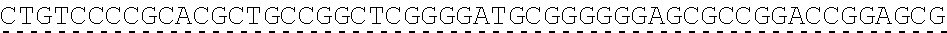

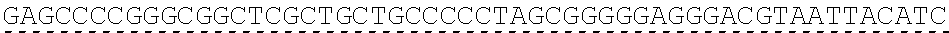

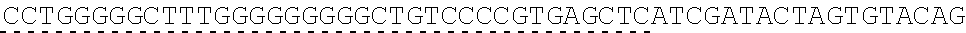

GTTAATGATGTTggctccggtgcccgtcagtgggcagagcgcacatcgcccacagtccccgagaagttggggggaggg
gtCggcaattgaaccggtgcctagagaaggtggcgcggggtaaactgggaaagtgatgtcgtgtactggctccgcctttttcccgag
ggtgggggagaaccgtatataagtgcagtagtcgccgtgaacgttctttttcgcaacgggtttgccgccagaacacaggtaagtgccg
tgtgtggttcccgcgggcctggcctctttacgggttatggcccttgcgtgccttgaattacttccacctggctgcagtacgtgattcttgatc
ccgagcttcgggttggaagtggggggagagttcgaggccttgcgcttaaggagccccttcgcctcgtgcttgagttgaggcctggcc
tgggcgctggggccgccgcgtgcgaatctggtggcaccttcgcgcctgtctcgctgctttcgataagtctctagccatttaaaatttttga
tgacctgctgcgacgctttttttctggcaagatagtcttgtaaatgcgggccaagatctgcacactggtatttcggtttttggggccgcggg
cggcgacggggcccgtgcgtcccagcgcacatgttcggcgaggcggggcctgcgagcgcggccaccgagaatcggacgggggt
agtctcaagctggccggcctgctctggtgcctggcctcgcgccgccgtgtatcgccccgccctgggcggcaaggctggcccggtcg
gcaccagttgcgtgagcggaaagatggccgcttcccggccctgctgcagggagctcaaaatggaggacgcggcgctcgggagag
cgggcgggtgagtcacccacacaaaggaaaagggcctttccgtcctcagccgtcgcttcatgtgactccacggagtaccgggcgcc
gtccaggcacctcgattagttctcgagcttttggagtacgtcgtctttaggttggggggaggggttttatgcgatggagtttccccacact
gagtgggtggagactgaagttaggccagcttggcacttgatgtaattctccttggaatttgccctttttgagtttggatcttggttcattctca
agcctcagacagtggttcaaagtttttttcttccatttcaggtgtcgtgaggaattaGGATCCATCGCCACCATGGCCT
TACCAGTGACCGCCTTGCTCCTGCCGCTGGCCTTGCTGCTCCACGCCGCCAGGCC

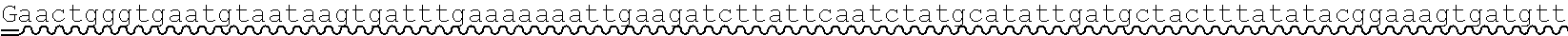

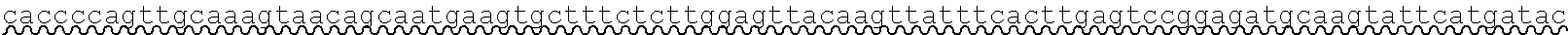

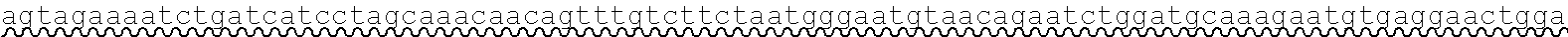

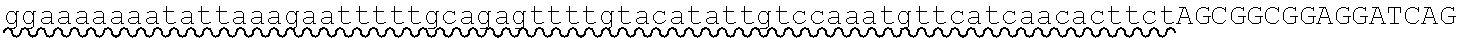

GTGGCGGTGGAAGCGGAGGTGGAGGCTCCGGTGGAGGAGGTAGTGGCGGAGGT
TCTCTTCAAatcacgtgccctccccccatgtccgtggaacacgcagacatctgggtcaagagctacagcttgtactccaggga
gcggtacatttgtaactctggtttcaagcgtaaagccggcacgtccagcctgacggagtgcgtgttgaacaaggccacgaatgtcgcc
cactggacaacccccagtctcaaatgcattagagaccctgccctggttcaccaaaggccagcgccaccctccacagtaacgacggca
ggggtgaccccacagccagagagcctctccccttctggaaaagagcccgcagcttcatctcccagctcaaacaacacageggccac
aacagcagctattgtcccgggctcccagctgatgccttcaaaatcaccttccacaggaaccacagagataagcagtcatgagtectcc
cacggcaccccctctcagacaacagccaagaactgggaactcacagcatccgcctcccaccagccgccaggtgtgtatccacaggg
ccacagcgacaccactgtggctatctccacgtccactgtcctgctgtgtgggctgagcgctgtgtctctcctggcatgctacctcaagtc
aaggcaaactcccccgctggccagcgttgaaatggaagccatggaggctctgccggtgacttgggggaccagcagcagagatgaa
gacttggaaaactgctctcaccacctaTGAatccctaggCGACTGTGCCTTCTAGTTGCCAGCCATCTG
TTGTTTGCCCCTCCCCCGTGCCTTCCTTGACCCTGGAAGGTGCCACTCCCACTGTC
CTTTCCTAATAAAATGAGGAAATTGCATCGCATTGTCTGAGTAGGTGTCATTCTA
TTCTGGGGGGGGGGTGGGGCAGGACAGCAAGGGGGAGGATTGGGAAGACAAT
AGCAGGCATGCTGGGGATGCGGTGGGCTCTATGGATAAACGCGGGGTTCGGTCC
CAGGGCTGGCACTCTGTCGATACCCCACCGAGACCCCATTGGGGCCAATACGCC
CGCGTTTCTTCCTTTTCCCCACCCCACCCCCCAAGTTCGGGTGAAGGCCCAGGGC

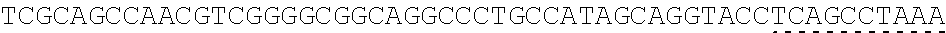

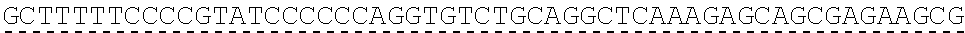

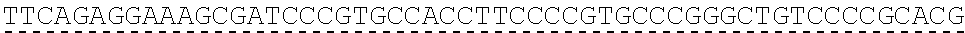

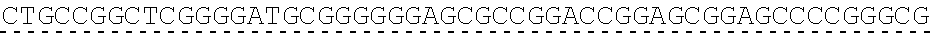

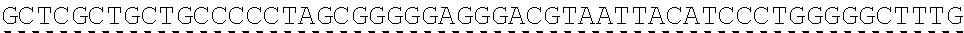

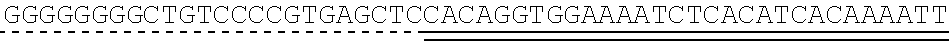

TACACAGTTGTGGAAACTTGACTGCACCGTTGTTGTCAGTGACTATCATGTCGTT
ATTAActgaggagagagaaagatatattaaatgattatccaactgccaggcagcctgccaatgaattcctgaagatgttatgcaattt
caaatgaacttgatgtcatgagaatgaatctgaagaaaggcaaaataattccttcacatcagattagaattatctggtgtatgcaatttgtaa
taaaatccattgtaccgtggtgaggggtggtgaggggttggggtggttgagtagccatttgcttttgtcaatggttgtcataaggtccactt
tctacaaaaatcttatcattcaataaagaaacctagatgggcccactgcatagtacggagtgctgggcccacaaataaaagtaccettaa
ggaaattatagtttagctggtgaataagacaggaaacagaccaagtcagggtgacccaaaatagagggcagcacagaggatgtggg
agccaggcgagcatcaccgctcagctgggcagggaggctataacctcacagagtcatgaagtgcca

An illustrative donor sequence for inserting a membrane-bound form of IL-15 into the TGFbR2 locus of a cell is shown in SEO ID NO: 30:

RHA(TGFbR2)-insul-EF1a-mbIL15-bGHpA-insul-LHA(TGFbR2)
1. RHA(TGFbR2 exon3-intron3): SEQ ID NO: 47

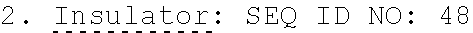

3. EFla (full length): SEQ ID NO: 49
4. CD8a (SP): 40

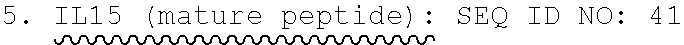

6. CD8a-hinge-TM: SEQ ID NO: 46
7. bGHpA: 51

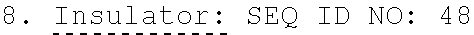

9. LHA (TGFbR2 intron2-exon3): SEQ ID NO: 52
(SEQ ID NO: 30)
tatatgaataatataaccagaatttctgactaaaaaaacaggttgtttttgtggttctccaagtagattccccatttttggctggtagttttctctt
ccaacagccaacctttcatcacctcttcttccagacctgagattgcctcaagagaacaaagctcctggtctcactatggggtgctgagga
ggtgtcggttaaatgactactaagcggcaaatccctcttcttgatacaagaaagagtttccagatttattatattaaagatcattttatgatctt
tacatttatcatgatcattttataatcatgatacgtttatatacccacataaacgtacacatacatgcagagaacacccctagaaactacattt
aataatcgaaagagagatggtctaaaggaaagggaaatggaacaggtgtttacatttaggagacagagatacactgactgtgtgtacta
tgagaatacattatgtaaaaaggggaaaagaaagaataacttcttaaaaggcttgcttacCATACAGCCACACAGACT
TCCTGTGGCTTCTCACAGATGGAGGTGATGCTGCAGTTGCTCATGCAGGATTTCT

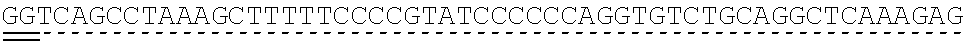

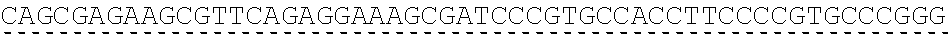

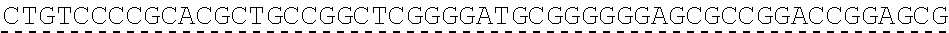

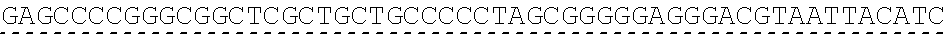

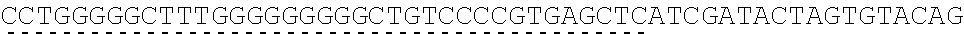

GTTAATGATGTTggctccggtgcccgtcagtgggcagagcgcacatcgcccacagtccccgagaagttggggggaggg
gtCggcaattgaaccggtgcctagagaaggtggcgcggggtaaactgggaaagtgatgtcgtgtactggctccgcctttttcccgag
ggtgggggagaaccgtatataagtgcagtagtcgccgtgaacgttctttttcgcaacgggtttgccgccagaacacaggtaagtgccg
tgtgtggttcccgcgggcctggcctctttacgggttatggcccttgcgtgccttgaattacttccacctggctgcagtacgtgattcttgatc
ccgagcttcgggttggaagtggggggagagttcgaggccttgcgcttaaggagccccttcgcctcgtgcttgagttgaggcctggcc
tgggcgctggggccgccgcgtgcgaatctggtggcaccttcgcgcctgtctcgctgctttcgataagtctctagccatttaaaatttttga
tgacctgctgcgacgctttttttctggcaagatagtcttgtaaatgcgggccaagatctgcacactggtatttcggtttttggggccgcggg
cggcgacggggcccgtgcgtcccagcgcacatgttcggcgaggcggggcctgcgagcgcggccaccgagaatcggacgggggt
agtctcaagctggccggcctgctctggtgcctggcctcgcgccgccgtgtatcgccccgccctgggcggcaaggctggcccggtcg
gcaccagttgcgtgagcggaaagatggccgcttcccggccctgctgcagggagctcaaaatggaggacgcggcgctcgggagag
cggggggtgagtcacccacacaaaggaaaagggcctttccgtcctcagccgtcgcttcatgtgactccacggagtaccgggcgcc
gtccaggcacctcgattagttctcgagcttttggagtacgtcgtctttaggttggggggaggggttttatgcgatggagtttccccacact
gagtgggtggagactgaagttaggccagcttggcacttgatgtaattctccttggaatttgccctttttgagtttggatcttggttcattctca
agcctcagacagtggttcaaagtttttttcttccatttcaggtgtcgtgaggaattaGGATCCATCGCCACCATGGCCT
TACCAGTGACCGCCTTGCTCCTGCCGCTGGCCTTGCTGCTCCACGCCGCCAGGCC

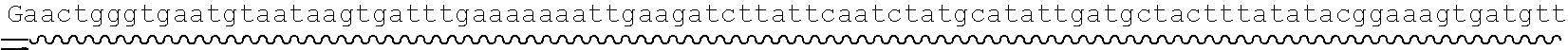

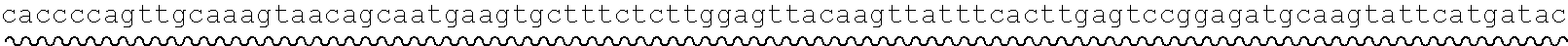

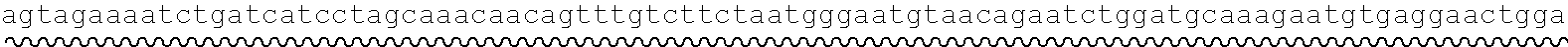

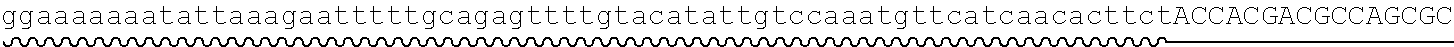

CGCGACCACCAACACCGGCGCCCACCATCGCGTCGCAGCCCCTGTCCCTGCGCCC
AGAGGCGTGCCGGCCAGCGGCGGGGGGCGCAGTGCACACGAGGGGGCTGGACT
TCGCCTGTGATATCTACATCTGGGCGCCCTTGGCCGGGACTTGTGGGGTCCTTCT
CCTGTCACTGGTTATCACCCTTTACTGCTGAatccctaggCGACTGTGCCTTCTAGTTG
CCAGCCATCTGTTGTTTGCCCCTCCCCCGTGCCTTCCTTGACCCTGGAAGGTGCCA
CTCCCACTGTCCTTTCCTAATAAAATGAGGAAATTGCATCGCATTGTCTGAGTAG
GTGTCATTCTATTCTGGGGGGGGGGTGGGGCAGGACAGCAAGGGGGAGGATTG
GGAAGACAATAGCAGGCATGCTGGGGATGCGGTGGGCTCTATGGATAAACGCGG
GGTTCGGTCCCAGGGCTGGCACTCTGTCGATACCCCACCGAGACCCCATTGGGGC
CAATACGCCCGCGTTTCTTCCTTTTCCCCACCCCACCCCCCAAGTTCGGGTGAAG

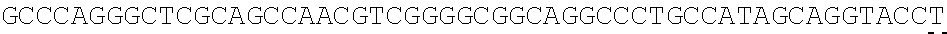

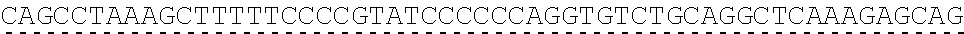

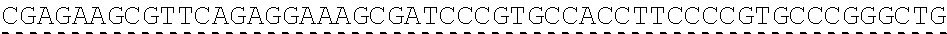

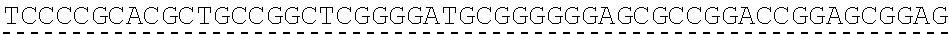

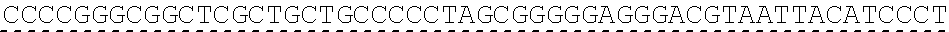

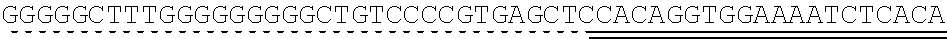

TCACAAAATTTACACAGTTGTGGAAACTTGACTGCACCGTTGTTGTCAGTGACTA
TCATGTCGTTATTAActgaggagagagaaagatatattaaatgattatccaactgccaggcagcctgccaatgaattcctg
aagatgttatgcaatttcaaatgaacttgatgtcatgagaatgaatctgaagaaaggcaaaataattccttcacatcagattagaattatctg
gtgtatgcaatttgtaataaaatccattgtaccgtggtgaggggtggtgaggggttggggtggttgagtagccatttgcttttgtcaatggt
tgtcataaggtccactttctacaaaaatcttatcattcaataaagaaacctagatgggcccactgcatagtacggagtgctgggcccaca
aataaaagtacccttaaggaaattatagtttagctggtgaataagacaggaaacagaccaagtcagggtgacccaaaatagagggca
gcacagaggatgtgggagccaggcgagcatcaccgctcagctgggcagggaggctataacctcacagagtcatgaagtgcca

An illustrative donor sequence for inserting an ER-trapped form of IL-15 fused to IL-15Rα into the B2M locus of a cell is shown in SEQ ID NO: 31:

LHA(B2M)-er[IL15-IL15Ra]-P2A-RHA(B2M)
1. LHA (B2M 5′upstream): SEQ ID NO: 39

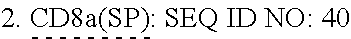

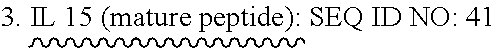

4. Spacer: SEQ ID NO: 42
5. IL15RA (mature peptide): SEQ ID NO: 43

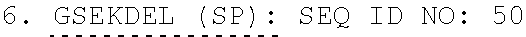

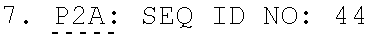

8. RHA (B2M exon1-intron1): SEQ ID NO: 45
(SEQIDNO:31)
gcaagaaaggtactctttcactaggaccttctctgagctgtcctcaggatgcttttgggactatttttcttacccagagaatggagaaaccc
tgcagggaattcccaagctgtagttataaacagaagttctccttctgctaggtagcattcaaagatcttaatcttctgggtttccgttttctcg
aatgaaaaatgcaggtccgagcagttaactggctggggcaccattagcaagtcacttagcatctctggggccagtctgcaaagcgag
ggggcagccttaatgtgcctccagcctgaagtcctagaatgagcgcccggtgtcccaagctggggcgcgcaccccagatcggagg
gcgccgatgtacagacagcaaactcacccagtctagtgcatgccttcttaaacatcacgagactctaagaaaaggaaactgaaaacgg
gaaagtccctctctctaacctggcactgcgtcgctggcttggagacaggtgacggtccctgcgggccttgtcctgattggctgggcac

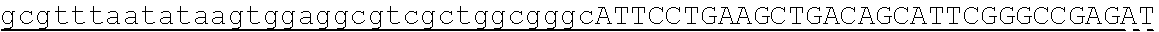

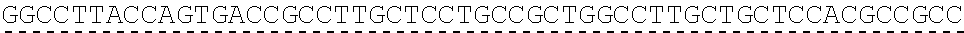

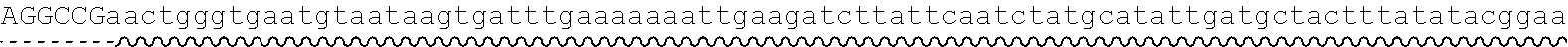

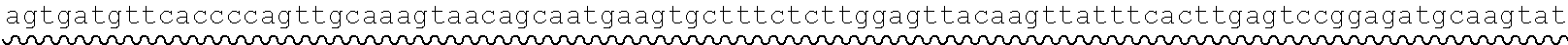

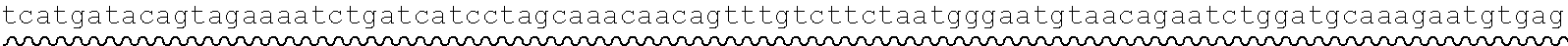

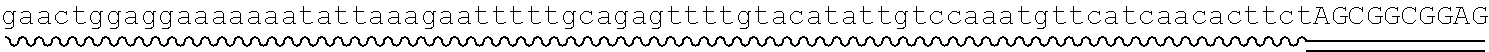

GATCAGGTGGCGGTGGAAGCGGAGGTGGAGGCTCCGGTGGAGGAGGTAGTGGC
GGAGGTTCTCTTCAAatcacgtgccctccccccatgtccgtggaacacgcagacatctgggtcaagagctacagcttgt
actccagggagcggtacatttgtaactctggtttcaagcgtaaagccggcacgtccagcctgacggagtgcgtgttgaacaaggccac
gaatgtcgcccactggacaacccccagtctcaaatgcattagagaccctgccctggttcaccaaaggccagegccaccctccacagt
aacgacggcaggggtgaccccacagccagagagcctctccccttctggaaaagagcccgcagcttcatctcccagctcaaacaaca
cagcggccacaacagcagctattgtcccgggctcccagctgatgccttcaaaatcaccttccacaggaaccacagagataagcagtc
atgagtcctcccacggcaccccctctcagacaacagccaagaactgggaactcacagcatccgcctcccaccagccgccaggtgtg
tatccacagggccacagcgacaccactgtggctatctccacgtccactgtcctgctgtgtgggctgagcgctgtgtctctcctggcatg
ctacctcaagtcaaggcaaactcccccgctggccagcgttgaaatggaagccatggaggctctgccggtgacttgggggaccagca

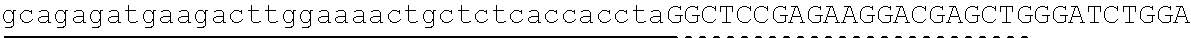

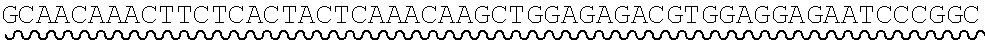

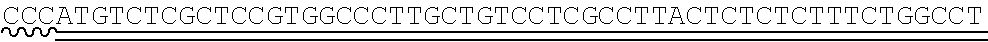

GGAGGCTATCCAGCgtgagtctctcctaccctcccgctctggtccttcctctcccgctctgcaccctctgtggccctcgctgt
gctctctcgctccgtgacttcccttctccaagttctccttggtggcccgccgtggggctagtccagggctggatctcggggaagcggcg
gggtggcctgggagtggggaagggggtgcgcacccgggacgcgcgctacttgcccctttcggcggggagcaggggagacctttg
gcctacggcgacgggagggtcgggacaaagtttagggcgtcgataagcgtcagagcgccgaggttggggggggtttctcttccgc
tctttcgcggggcctctggctcccccagcgcagctggagtgggggacgggtaggctcgtcccaaaggcgcggcgctgaggtttgtg
aacgcgtggaggggcgcttggggtctgggggaggcgtcgcccgggtaagcctgtctgctgcggctctgcttcccttagactggaga
gctgtggacttcgtctaggcgcccgctaagttc

An illustrative donor sequence for inserting an ER-trapped form of IL-15 into the B2M locus of a cell is shown in SEQ ID NO: 32:

LHA(B2M)-erIL15-P2A-RHA(B2M)
1. LHA(B2M 5′upstream): SEQ ID NO: 39

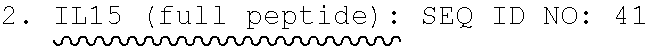

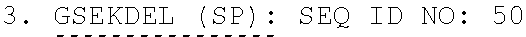

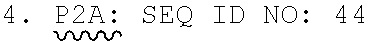

5. RHA (B2M exon1-intron1): SEQ ID NO: 45
(SEQ ID NO: 32)
gcaagaaaggtactctttcactaggaccttctctgagctgtcctcaggatgcttttgggactatttttcttacccagagaatggagaaaccc
tgcagggaattcccaagctgtagttataaacagaagttctccttctgctaggtagcattcaaagatcttaatcttctgggtttccgttttctcg
aatgaaaaatgcaggtccgagcagttaactggctggggcaccattagcaagtcacttagcatctctggggccagtctgcaaagcgag
ggggcagccttaatgtgcctccagcctgaagtcctagaatgagcgcccggtgtcccaagctggggcgcgcaccccagatcggagg
gcgccgatgtacagacagcaaactcacccagtctagtgcatgccttcttaaacatcacgagactctaagaaaaggaaactgaaaacgg
gaaagtccctctctctaacctggcactgcgtcgctggcttggagacaggtgacggtccctgcgggccttgtcctgattggctgggcac

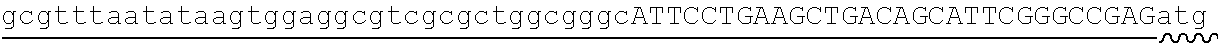

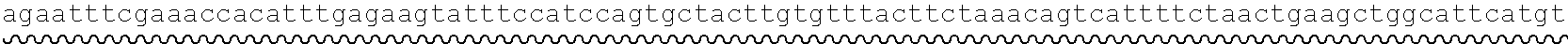

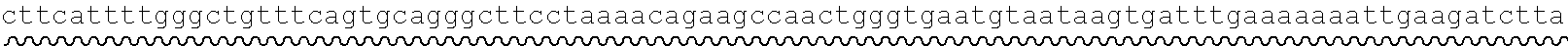

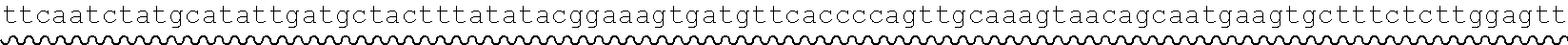

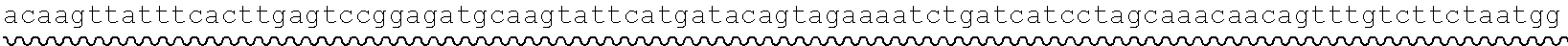

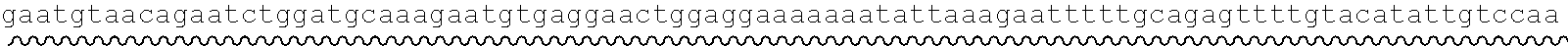

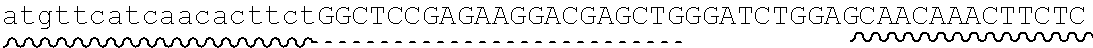

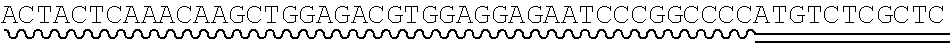

CGTGGCCCTTGCTGTCCTCGCCTTACTCTCTCTTTCTGGCCTGGAGGCTATCCAGC
gtgagtctctcctaccctcccgctctggtccttcctctcccgctctgcaccctctgtggccctcgctgtgctctctcgctccgtgacttccct
tctccaagttctccttggtggcccgccgtggggctagtccagggctggatctcggggaagcggcggggggcctgggagtggggaa
gggggtgcgcacccgggacgcgcgctacttgcccctttcggcggggagcaggggagacctttggcctacggcgacgggagggtc
gggacaaagtttagggcgtcgataagcgtcagagcgccgagottggggggggtttctcttccgctctttcgcggggcctctggctcc
cccagcgcagctggagtgggggacgggtaggctcgtcccaaaggcgcggcgctgaggtttgtgaacgcgtggaggggcgcttgg
ggtctgggggaggcgtcgcccgggtaagcctgtctgctgcggctctgcttcccttagactggagagctgtggacttcgtctaggcgcc
cgctaagttc

An illustrative donor sequence for inserting a membrane-bound form of IL-15 into the B2M locus of a cell is shown in SEQ ID NO: 33:

LHA(B2M)-mbIL15-IRES-RHA(B2M)
1. LHA(B2M 5′upstream): SEQ ID NO: 39

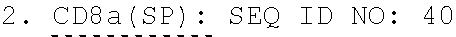

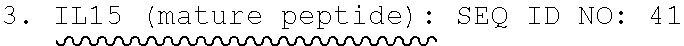

4. CD8a-hinge-TM: SEQ ID NO: 46

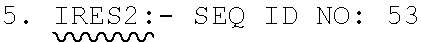

6. RHA (B2M exon1-intron1): SEQ ID NO: 45
(SEQ ID NO: 33)
gcaagaaaggtactctttcactaggaccttctctgagctgtcctcaggatgcttttgggactatttttcttacccagagaatggagaaaccc
tgcagggaattcccaagctgtagttataaacagaagttctccttctgctaggtagcattcaaagatcttaatcttctgggtttccgttttctcg
aatgaaaaatgcaggtccgagcagttaactggctggggcaccattagcaagtcacttagcatctctggggccagtctgcaaagcgag
ggggcagccttaatgtgcctccagcctgaagtcctagaatgagcgcccggtgtcccaagctggggcgcgcaccccagatcggagg
gcgccgatgtacagacagcaaactcacccagtctagtgcatgccttcttaaacatcacgagactctaagaaaaggaaactgaaaacgg
gaaagtccctctctctaacctggcactgcgtcgctggcttggagacaggtgacggtccctgcgggccttgtcctgattggctgggcac

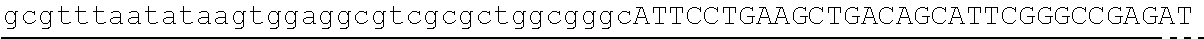

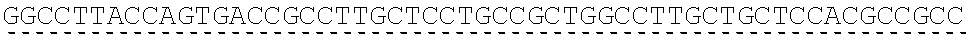

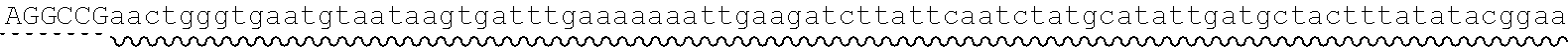

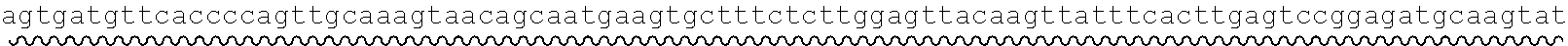

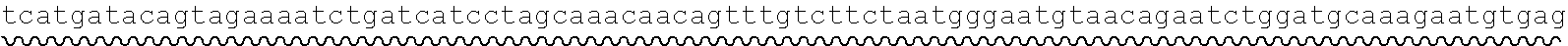

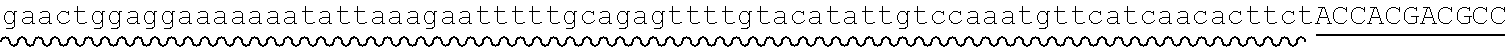

AGCGCCGCGACCACCAACACCGGCGCCCACCATCGCGTCGCAGCCCCTGTCCCT
GCGCCCAGAGGCGTGCCGGCCAGCGGCGGGGGGCGCAGTGCACACGAGGGGGC
TGGACTTCGCCTGTGATATCTACATCTGGGCGCCCTTGGCCGGGACTTGTGGGGT
CCTTCTCCTGTCACTGGTTATCACCCTTTACTGCTGAAATTCCGCCCCTCTCCCCC

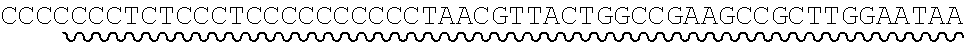

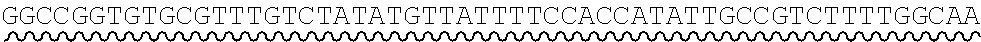

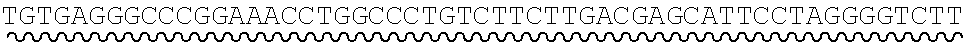

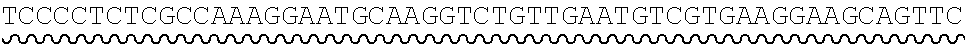

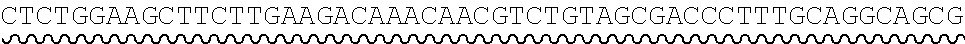

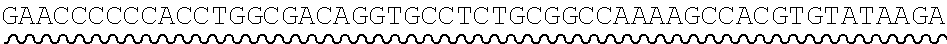

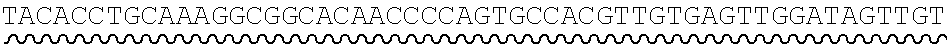

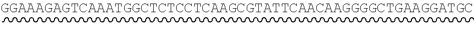

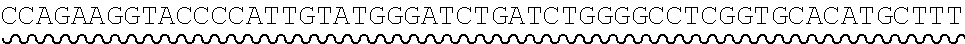

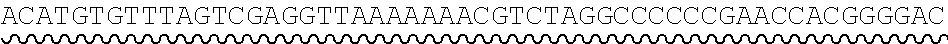

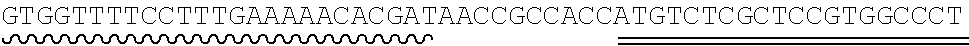

TGCTGTCCTCGCCTTACTCTCTCTTTCTGGCCTGGAGGCTATCCAGCgtgagtctctcctac
cctcccgctctggtccttcctctcccgctctgcaccctctgtggccctcgctgtgctctctcgctccgtgacttcccttctccaagttctcctt
ggtggcccgccgtggggctagtccagggctggatctcggggaagcggcggggtggcctgggagtggggaaggggggtgcgcacc
cgggacgcgcgctacttgcccctttcggcggggagcaggggagacctttggcctacggcgacgggagggtcgggacaaagtttag
ggcgtcgataagcgtcagagcgccgagottgggggagggtttctcttccgctctttcgcggggcctctggctcccccagcgcagctg
gagtgggggacgggtaggctcgtcccaaaggcgcggcgctgaggtttgtgaacgcgtggaggggcgcttggggtctgggggagg
cgtcgcccgggtaagcctgtctgctgcggctctgcttcccttagactggagagctgtggacttcgtctaggcgcccgctaagttc

An illustrative donor sequence for inserting IL-15 fused to IL-15Rα into the B2M locus of a cell is shown in SEQ ID NO: 34:

LHA(B2M)-IL15-IL15Ra-IRES-RHA(B2M)
1. LHA(B2M 5′upstream): SEQ ID NO: 39
2. CD8a(SP): SEQ ID NO: 40
3. 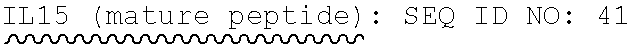
4. 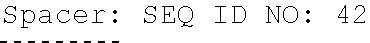
5. IL15RA (mature peptide): SEQ ID NO: 43
6. 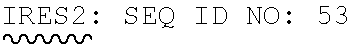
7. RHA (B2M exon1-intron1): SEQ ID NO: 45
(SEQ ID NO: 34)
gcaagaaaggtactctttcactaggaccttctctgagctgtcctcaggatgcttttgggactatttttcttacccagagaatggagaaaccc
tgcagggaattcccaagctgtagttataaacagaagttctccttctgctaggtagcattcaaagatcttaatcttctgggtttccgttttctcg
aatgaaaaatgcaggtccgagcagttaactggctggggcaccattagcaagtcacttagcatctctggggccagtctgcaaagcgag
ggggcagccttaatgtgcctccagcctgaagtcctagaatgagcgcccggtgtcccaagctggggcgcgcaccccagatcggagg
gcgccgatgtacagacagcaaactcacccagtctagtgcatgccttcttaaacatcacgagactctaagaaaaggaaactgaaaacgg
gaaagtccctctctctaacctggcactgcgtcgctggcttggagacaggtgacggtccctgcgggccttgtcctgattggctgggcac
gcgtttaatataagtggaggcgtcgcgctggcgggcATTCCTGAAGCTGACAGCATTCGGGCCGAGAT
GGCCTTACCAGTGACCGCCTTGCTCCTGCCGCTGGCCTTGCTGCTCCACGCCGCC

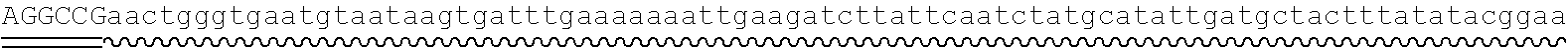

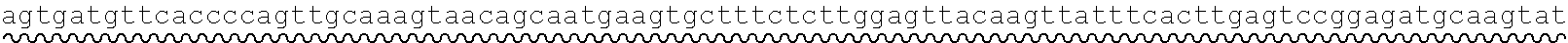

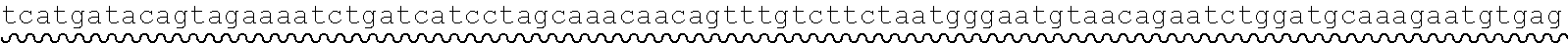

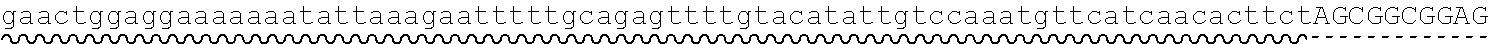

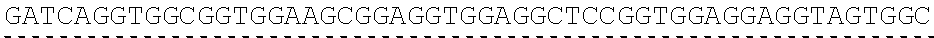

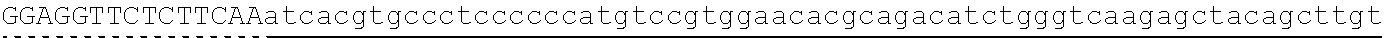

actccagggagcggtacatttgtaactctggtttcaagcgtaaagccggcacgtccagcctgacggagtgcgtgttgaacaaggccac
gaatgtcgcccactggacaacccccagtctcaaatgcattagagaccctgccctggttcaccaaaggccagegccaccctccacagt
aacgacggcaggggtgaccccacagccagagagcctctccccttctggaaaagagcccgcagcttcatctcccagctcaaacaaca
cagcggccacaacagcagctattgtcccgggctcccagctgatgccttcaaaatcaccttccacaggaaccacagagataagcagtc
atgagtcctcccacggcaccccctctcagacaacagccaagaactgggaactcacagcatccgcctcccaccagccgccaggtgtg
tatccacagggccacagcgacaccactgtggctatctccacgtccactgtcctgctgtgtgggctgagcgctgtgtctctcctggcatg
ctacctcaagtcaaggcaaactcccccgctggccagcgttgaaatggaagccatggaggctctgccggtgacttgggggaccagca

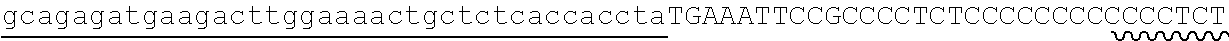

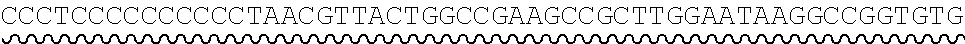

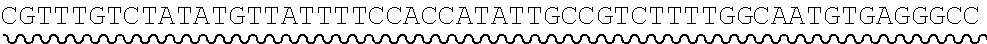

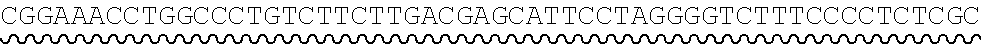

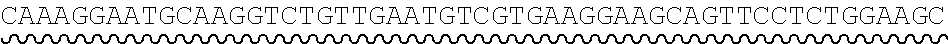

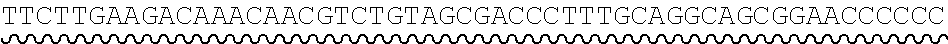

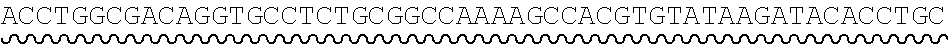

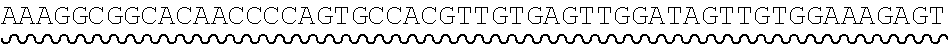

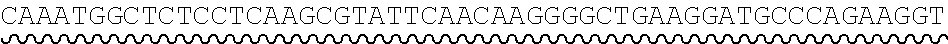

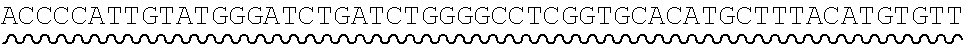

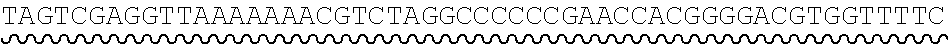

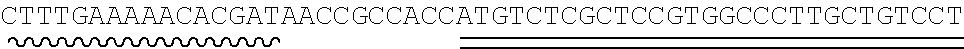

CGCCTTACTCTCTCTTTCTGGCCTGGAGGCTATCCAGCgtgagtctctcctaccctcccgctctggtc
cttcctctcccgctctgcaccctctgtggccctcgctgtgctctctcgctccgtgacttcccttctccaagttctccttggtggcccgccgtg
gggctagtccagggctggatctcggggaagcggcggggtggcctgggagtggggaagggggtgcgcacccgggacgcgcgcta
cttgcccctttcggcggggagcaggggagacctttggcctacggcgacgggagggtcgggacaaagtttagggcgtcgataagcgt
cagagcgccgaggttgggggagggtttctcttccgctctttcgcggggcctctggctcccccagcgcagctggagtgggggacggg
taggctcgtcccaaaggcgcggcgctgaggtttgtgaacgcgtggaggggcgcttggggtctgggggaggcgtcgcccgggtaag
cctgtctgctgcggctctgcttcccttagactggagagctgtggacttcgtctaggcgcccgctaagttc

An illustrative donor sequence for inserting an ER-trapped form of IL-15 fused to IL-15Rα into the TGFbR2 locus of a cell is shown in SEQ ID NO: 35:

RHA(TGFbR2)-insul-EF1a-er[IL15-IL15Ra]-bGHpA-insul-LHA(TGFbR2)
1. RHA(TGFbR2 exon3-intron3): SEQ ID NO: 47
2. 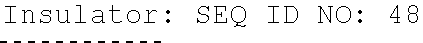
3. EFla (full length): SEQ ID NO:49
4. CD8a (SP): 40
5. 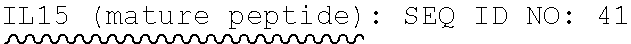
6. 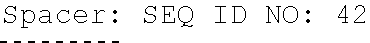
7. IL15RA (mature peptide): SEQ ID NO: 43
8. GSEKDEL (SP): SEQ ID NO: 50
9. bGHpA: 51
10. 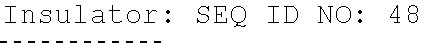
11. LHA (TGFbR2 intron2-exon3): SEQ ID NO: 52
(SEQ ID NO: 35)
tatatgaataatataaccagaatttctgactaaaaaaacaggttgtttttgtggttctccaagtagattccccatttttggctggtagttttctctt
ccaacagccaacctttcatcacctcttcttccagacctgagattgcctcaagagaacaaagctcctggtctcactatggggtgctgagga
ggtgtcggttaaatgactactaagcggcaaatccctcttcttgatacaagaaagagtttccagatttattatattaaagatcattttatgatctt
tacatttatcatgatcattttataatcatgatacgtttatatacccacataaacgtacacatacatgcagagaacacccctagaaactacattt
aataatcgaaagagagatggtctaaaggaaagggaaatggaacaggtgtttacatttaggagacagagatacactgactgtgtgtacta
tgagaatacattatgtaaaaaggggaaaagaaagaataacttcttaaaaggcttgcttacCATACAGCCACACAGACT
TCCTGTGGCTTCTCACAGATGGAGGTGATGCTGCAGTTGCTCATGCAGGATTTCT

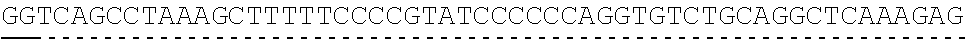

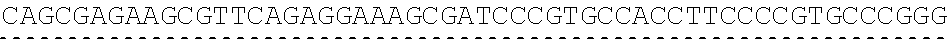

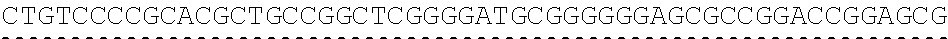

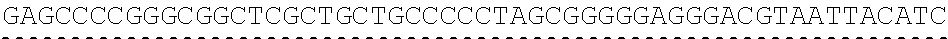

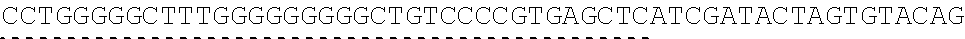

GTTAATGATGTTggctccggtgcccgtcagtgggcagagcgcacatcgcccacagtccccgagaagttggggggaggg
gtCggcaattgaaccggtgcctagagaaggtggcgcggggtaaactgggaaagtgatgtcgtgtactggctccgcctttttcccgag
ggtgggggagaaccgtatataagtgcagtagtcgccgtgaacgttctttttcgcaacgggtttgccgccagaacacaggtaagtgccg
tgtgtggttcccgcgggcctggcctctttacgggttatggcccttgcgtgccttgaattacttccacctggctgcagtacgtgattcttgatc
ccgagcttcgggttggaagtggggggagagttcgaggccttgcgcttaaggagccccttcgcctcgtgcttgagttgaggcctggcc
tgggcgctggggccgccgcgtgcgaatctggtggcaccttcgcgcctgtctcgctgctttcgataagtctctagccatttaaaatttttga
tgacctgctgcgacgctttttttctggcaagatagtcttgtaaatgcgggccaagatctgcacactggtatttcggtttttggggccgcggg
cggcgacggggcccgtgcgtcccagcgcacatgttcggcgaggcggggcctgcgagcgcggccaccgagaatcggacgggggt
agtctcaagctggccggcctgctctggtgcctggcctcgcgccgccgtgtatcgccccgccctgggcggcaaggctggcccggtcg
gcaccagttgcgtgagcggaaagatggccgcttcccggccctgctgcagggagctcaaaatggaggacgcggcgctcgggagag
cgggcgggtgagtcacccacacaaaggaaaagggcctttccgtcctcagccgtcgcttcatgtgactccacggagtaccgggcgcc
gtccaggcacctcgattagttctcgagcttttggagtacgtcgtctttaggttggggggaggggttttatgcgatggagtttccccacact
gagtgggtggagactgaagttaggccagcttggcacttgatgtaattctccttggaatttgccctttttgagtttggatcttggttcattctca
agcctcagacagtggttcaaagtttttttcttccatttcaggtgtcgtgaggaattaGGATCCATCGCCACCATGGCCT
TACCAGTGACCGCCTTGCTCCTGCCGCTGGCCTTGCTGCTCCACGCCGCCAGGCC

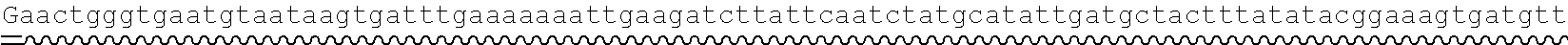

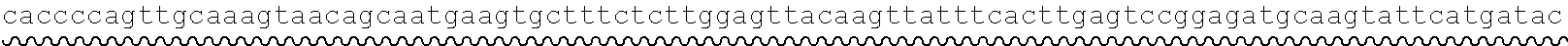

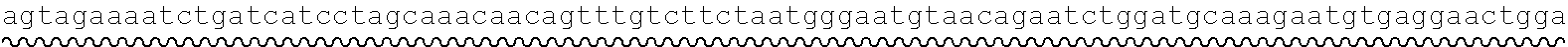

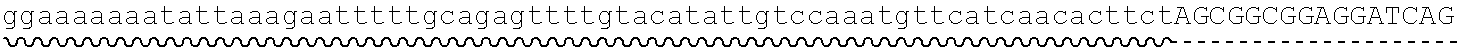

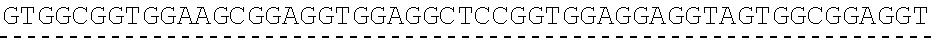

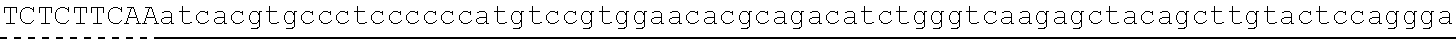

gcggtacatttgtaactctggtttcaagcgtaaagccggcacgtccagcctgacggagtgcgtgttgaacaaggccacgaatgtcgcc
cactggacaacccccagtctcaaatgcattagagaccctgccctggttcaccaaaggccagcgccaccctccacagtaacgacggca
ggggtgaccccacagccagagagcctctccccttctggaaaagagcccgcagcttcatctcccagctcaaacaacacagcggccac
aacagcagctattgtcccgggctcccagctgatgccttcaaaatcaccttccacaggaaccacagagataagcagtcatgagtcctcc
cacggcaccccctctcagacaacagccaagaactgggaactcacagcatccgcctcccaccagccgccaggtgtgtatccacaggg
ccacagcgacaccactgtggctatctccacgtccactgtcctgctgtgtgggctgagcgctgtgtctctcctggcatgctacctcaagtc
aaggcaaactcccccgctggccagcgttgaaatggaagccatggaggctctgccggtgacttgggggaccagcagcagagatgaa
gacttggaaaactgctctcaccacctaGGCTCCGAGAAGGACGAGCTGTGAatccctaggCGACTGTG
CCTTCTAGTTGCCAGCCATCTGTTGTTTGCCCCTCCCCCGTGCCTTCCTTGACCCT
GGAAGGTGCCACTCCCACTGTCCTTTCCTAATAAAATGAGGAAATTGCATCGCAT
TGTCTGAGTAGGTGTCATTCTATTCTGGGGGGTGGGGGGGGCAGGACAGCAAG
GGGGAGGATTGGGAAGACAATAGCAGGCATGCTGGGGATGCGGTGGGCTCTATG
GATAAACGCGGGGTTCGGTCCCAGGGCTGGCACTCTGTCGATACCCCACCGAGA
CCCCATTGGGGCCAATACGCCCGCGTTTCTTCCTTTTCCCCACCCCACCCCCCAAG
TTCGGGTGAAGGCCCAGGGCTCGCAGCCAACGTCGGGGCGGCAGGCCCTGCCAT

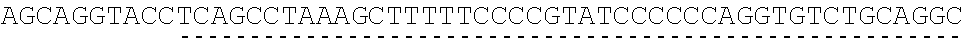

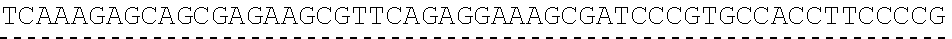

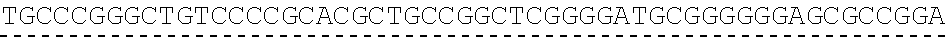

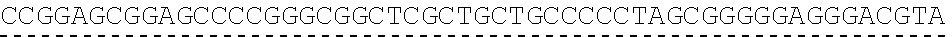

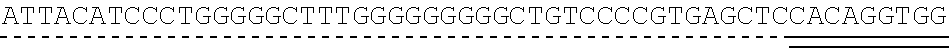

AAAATCTCACATCACAAAATTTACACAGTTGTGGAAACTTGACTGCACCGTTGTT
GTCAGTGACTATCATGTCGTTATTAActgaggagagagaaagatatattaaatgattatccaactgccaggca
gcctgccaatgaattcctgaagatgttatgcaatttcaaatgaacttgatgtcatgagaatgaatctgaagaaaggcaaaataattccttca
catcagattagaattatctggtgtatgcaatttgtaataaaatccattgtaccgtggtgaggggtggtgaggggttggggtggttgagtag
ccatttgcttttgtcaatggttgtcataaggtccactttctacaaaaatcttatcattcaataaagaaacctagatgggcccactgcatagtac
ggagtgctgggcccacaaataaaagtacccttaaggaaattatagtttagctggtgaataagacaggaaacagaccaagtcagggtga
cccaaaatagagggcagcacagaggatgtgggagccaggcgagcatcaccgctcagctgggcagggaggctataacctcacaga
gtcatgaagtgcca

An illustrative donor sequence for inserting an ER-trapped form of IL-15 into the TGFbR2 locus of a cell is shown in SEQ ID NO: 36:

RHA(TGFbR2)-insul-EF1a-erIL15-bGHpA-insul-LHA(TGFbR2)
1. RHA(TGFbR2 exon3-intron3): SEQ ID NO: 47
2. 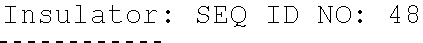
3. EFla (full length): SEQ ID NO: 49
4. 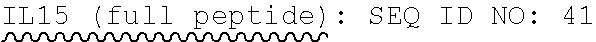
5. GSEKDEL (SP): SEQ ID NO: 50
6. bGHpA: 51
7. 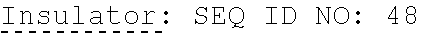
8. LHA (TGFbR2 intron2-exon3): SEQ ID NO: 52
(SEQ ID NO: 36)
tatatgaataatataaccagaatttctgactaaaaaaacaggttgtttttgtggttctccaagtagattccccatttttggctggtagttttctctt
ccaacagccaacctttcatcacctcttcttccagacctgagattgcctcaagagaacaaagctcctggtctcactatggggtgctgagga
ggtgtcggttaaatgactactaagcggcaaatccctcttcttgatacaagaaagagtttccagatttattatattaaagatcattttatgatctt
tacatttatcatgatcattttataatcatgatacgtttatatacccacataaacgtacacatacatgcagagaacacccctagaaactacattt
aataatcgaaagagagatggtctaaaggaaagggaaatggaacaggtgtttacatttaggagacagagatacactgactgtgtgtacta
tgagaatacattatgtaaaaaggggaaaagaaagaataacttcttaaaaggcttgcttacCATACAGCCACACAGACT
TCCTGTGGCTTCTCACAGATGGAGGTGATGCTGCAGTTGCTCATGCAGGATTTCT

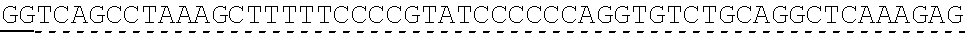

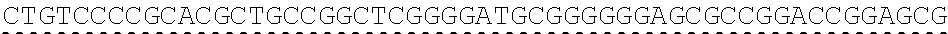

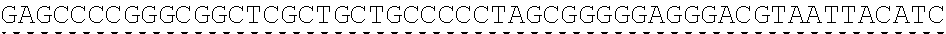

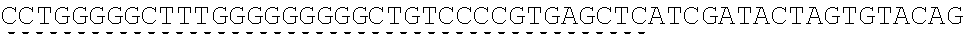

GTTAATGATGTTggctccggtgcccgtcagtgggcagagcgcacatcgcccacagtccccgagaagttggggggaggg
gtCggcaattgaaccggtgcctagagaaggtggcgcggggtaaactgggaaagtgatgtcgtgtactggctccgcctttttcccgag
ggtgggggagaaccgtatataagtgcagtagtcgccgtgaacgttctttttcgcaacgggtttgccgccagaacacaggtaagtgccg
tgtgtggttcccgcgggcctggcctctttacgggttatggcccttgcgtgccttgaattacttccacctggctgcagtacgtgattcttgatc
ccgagcttcgggttggaagtgggtgggagagttcgaggccttgcgcttaaggagccccttcgcctcgtgcttgagttgaggcctggcc
tgggcgctggggccgccgcgtgcgaatctggtggcaccttcgcgcctgtctcgctgctttcgataagtctctagccatttaaaatttttga
tgacctgctgcgacgctttttttctggcaagatagtcttgtaaatgcgggccaagatctgcacactggtatttcggtttttggggccgcggg
cggcgacggggcccgtgcgtcccagcgcacatgttcggcgaggcggggcctgcgagcgcggccaccgagaatcggacgggggt
agtctcaagctggccggcctgctctggtgcctggcctcgcgccgccgtgtatcgccccgccctgggcggcaaggctggcccggtcg
gcaccagttgcgtgagcggaaagatggccgcttcccggccctgctgcagggagctcaaaatggaggacgcggcgctcgggagag
cgggcgggtgagtcacccacacaaaggaaaagggcctttccgtcctcagccgtcgcttcatgtgactccacggagtaccgggcgcc
gtccaggcacctcgattagttctcgagcttttggagtacgtcgtctttaggttggggggaggggttttatgcgatggagtttccccacact
gagtgggtggagactgaagttaggccagcttggcacttgatgtaattctccttggaatttgccctttttgagtttggatcttggttcattctca

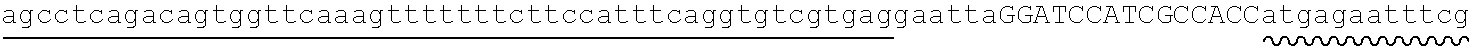

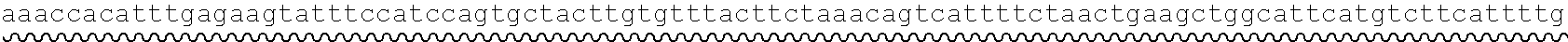

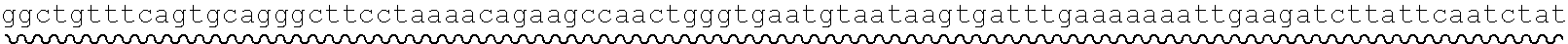

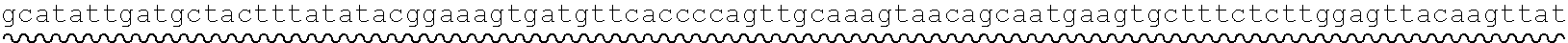

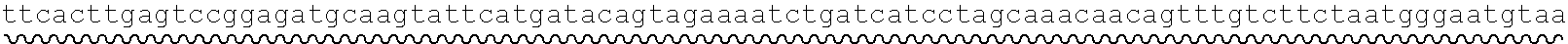

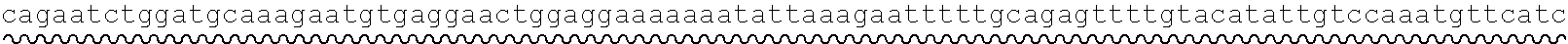

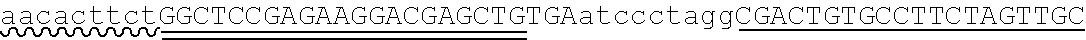

CAGCCATCTGTTGTTTGCCCCTCCCCCGTGCCTTCCTTGACCCTGGAAGGTGCCAC
TCCCACTGTCCTTTCCTAATAAAATGAGGAAATTGCATCGCATTGTCTGAGTAGG
TGTCATTCTATTCTGGGGGGTGGGGTGGGGCAGGACAGCAAGGGGGAGGATTGG
GAAGACAATAGCAGGCATGCTGGGGATGCGGTGGGCTCTATGGATAAACGCGGG
GTTCGGTCCCAGGGCTGGCACTCTGTCGATACCCCACCGAGACCCCATTGGGGCC
AATACGCCCGCGTTTCTTCCTTTTCCCCACCCCACCCCCCAAGTTCGGGTGAAGG

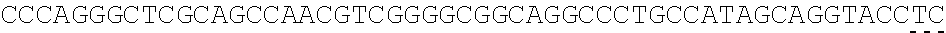

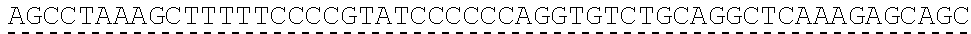

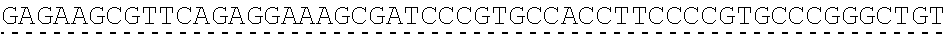

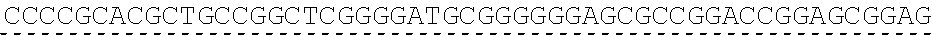

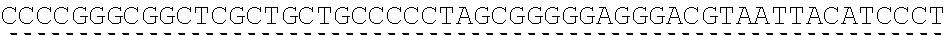

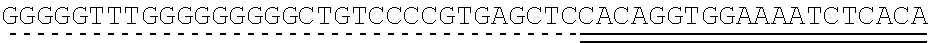

TCACAAAATTTACACAGTTGTGGAAACTTGACTGCACCGTTGTTGTCAGTGACTA
TCATGTCGTTATTAActgaggagagagaaagatatattaaatgattatccaactgccaggcagcctgccaatgaattcctg
aagatgttatgcaatttcaaatgaacttgatgtcatgagaatgaatctgaagaaaggcaaaataattccttcacatcagattagaattatctg
gtgtatgcaatttgtaataaaatccattgtaccgtggtgaggggtggtgaggggttggggtggttgagtagccatttgcttttgtcaatggt
tgtcataaggtccactttctacaaaaatcttatcattcaataaagaaacctagatgggcccactgcatagtacggagtgctgggcccaca
aataaaagtacccttaaggaaattatagtttagctggtgaataagacaggaaacagaccaagtcagggtgacccaaaatagagggca
gcacagaggatgtgggagccaggcgagcatcaccgctcagctgggcagggaggctataacctcacagagtcatgaagtgcca

An illustrative donor sequence for inserting an ER-trapped form of IL-15 fused to IL-15Rα into the B2M locus of a cell is shown in SEQ ID NO: 37:

LHA(B2M)-er[IL15-IL15Ra]-IRES-RHA(B2M)
1. LHA (B2M 5′upstream): SEQ ID NO: 39
2. CD8a(SP): SEQ ID NO: 40
3. 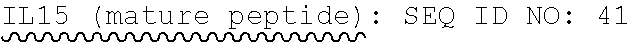
4. 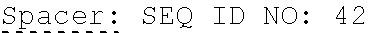
5. IL15RA (mature peptide): SEQ ID NO: 43
6. 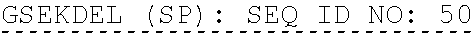
7. IRES2: SEQ ID NO: 53
8. RHA (B2M exon1-intron1): SEQ ID NO: 45
(SEQ ID NO: 37)
gcaagaaaggtactctttcactaggaccttctctgagctgtcctcaggatgcttttgggactatttttcttacccagagaatggagaaaccc
tgcagggaattcccaagctgtagttataaacagaagttctccttctgctaggtagcattcaaagatcttaatcttctgggtttccgttttctcg
aatgaaaaatgcaggtccgagcagttaactggctggggcaccattagcaagtcacttagcatctctggggccagtctgcaaagcgag
ggggcagccttaatgtgcctccagcctgaagtcctagaatgagcgcccggtgtcccaagctggggcgcgcaccccagatcggagg
gcgccgatgtacagacagcaaactcacccagtctagtgcatgccttcttaaacatcacgagactctaagaaaaggaaactgaaaacgg
gaaagtccctctctctaacctggcactgcgtcgctggcttggagacaggtgacggtccctgcgggccttgtcctgattggctgggcac
gcgtttaatataagtggaggcgtcgcgctggcgggcATTCCTGAAGCTGACAGCATTCGGGCCGAGAT
GGCCTTACCAGTGACCGCCTTGCTCCTGCCGCTGGCCTTGCTGCTCCACGCCGCC

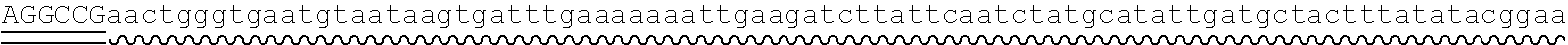

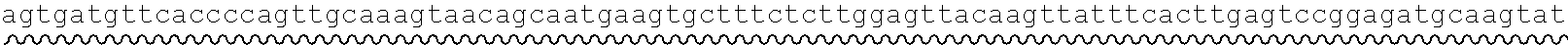

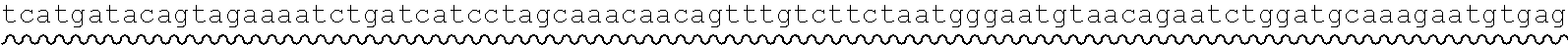

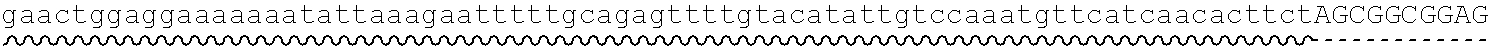

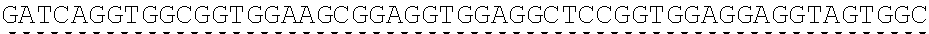

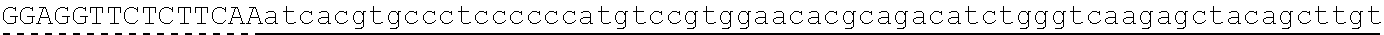

actccagggagcggtacatttgtaactctggtttcaagcgtaaagccggcacgtccagcctgacggagtgcgtgttgaacaaggccac
gaatgtcgcccactggacaacccccagtctcaaatgcattagagaccctgccctggttcaccaaaggccagcgccaccctccacagt
aacgacggcaggggtgaccccacagccagagagcctctccccttctggaaaagagcccgcagcttcatctcccagctcaaacaaca
cagcggccacaacagcagctattgtcccgggctcccagctgatgccttcaaaatcaccttccacaggaaccacagagataagcagtc
atgagtcctcccacggcaccccctctcagacaacagccaagaactgggaactcacagcatccgcctcccaccagccgccaggtgtg
tatccacagggccacagcgacaccactgtggctatctccacgtccactgtcctgctgtgtgggctgagcgctgtgtctctcctggcatg
ctacctcaagtcaaggcaaactcccccgctggccagcgttgaaatggaagccatggaggctctgccggtgacttgggggaccagca

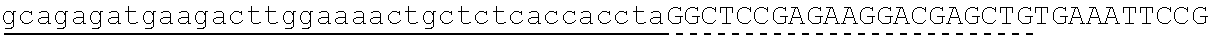

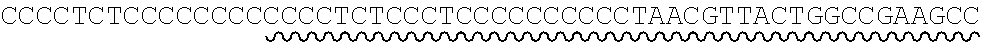

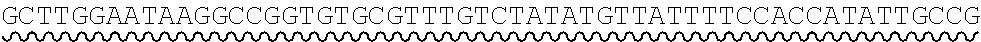

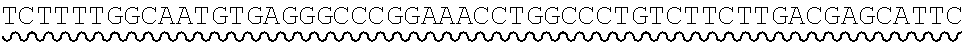

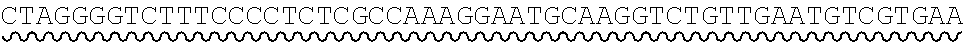

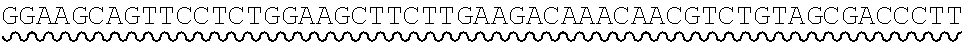

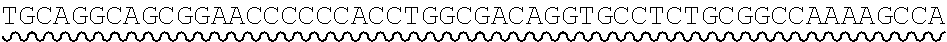

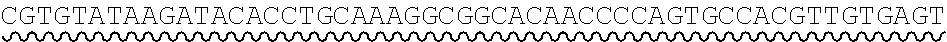

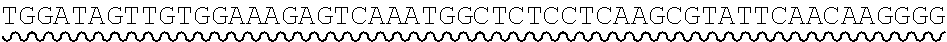

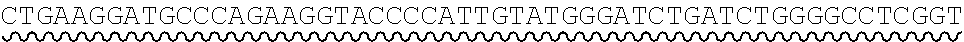

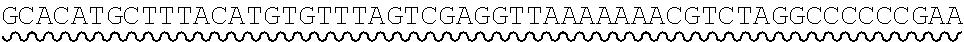

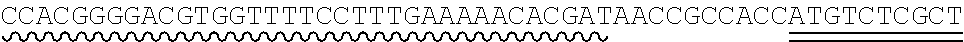

CCGTGGCCCTTGCTGTCCTCGCCTTACTCTCTCTTTCTGGCCTGGAGGCTATCCAG
Cgtgagtctctcctaccctcccgctctggtccttcctctcccgctctgcaccctctgtggccctcgctgtgctctctcgctccgtgacttcc
cttctccaagttctccttggtggcccgccgtggggctagtccagggctggatctcggggaagcggcggggtogcctgggagtgggg
aagggggtgcgcacccgggacgcgcgctacttgcccctttcggcggggagcaggggagacctttggcctacggcgacgggaggg
tcgggacaaagtttagggcgtcgataagcgtcagagcgccgagottggggggggtttctcttccgctctttcgcggggcctctggct
cccccagcgcagctggagtgggggacgggtaggctcgtcccaaaggcgcggcgctgaggtttgtgaacgcgtggaggggcgctt
ggggtctgggggaggcgtcgcccgggtaagcctgtctgctgcggctctgcttcccttagactggagagctgtggacttcgtctaggcg
cccgctaagttc

An illustrative donor sequence for inserting an ER-trapped form of IL-15 into the B2M locus of a cell is shown in SEQ ID NO: 38:

LHA(B2M)-erIL15-IRES-RHA(B2M)
1. LHA (B2M 5′upstream): SEQ ID NO: 39
2. 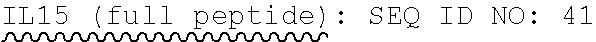
3. 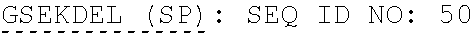
4. IRES2: SEQ ID NO: 53
5. RHA (B2M exon1-intron1): SEQ ID NO: 45
(SEQ ID NO: 38)
gcaagaaaggtactctttcactaggaccttctctgagctgtcctcaggatgcttttgggactatttttcttacccagagaatggagaaaccc
tgcagggaattcccaagctgtagttataaacagaagttctccttctgctaggtagcattcaaagatcttaatcttctgggtttccgttttctcg
aatgaaaaatgcaggtccgagcagttaactggctggggcaccattagcaagtcacttagcatctctggggccagtctgcaaagcgag
ggggcagccttaatgtgcctccagcctgaagtcctagaatgagcgcccggtgtcccaagctggggcgcgcaccccagatcggagg
gcgccgatgtacagacagcaaactcacccagtctagtgcatgccttcttaaacatcacgagactctaagaaaaggaaactgaaaacgg
gaaagtccctctctctaacctggcactgcgtcgctggcttggagacaggtgacggtccctgcgggccttgtcctgattggctgggcac

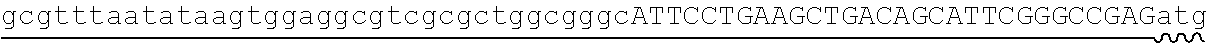

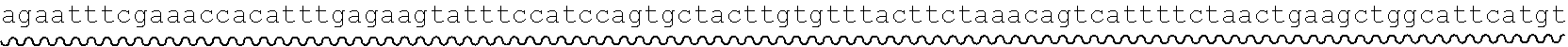

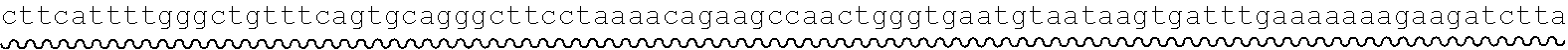

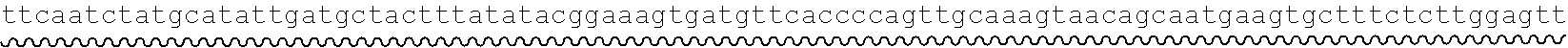

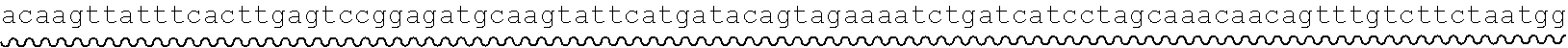

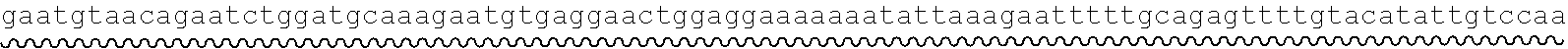

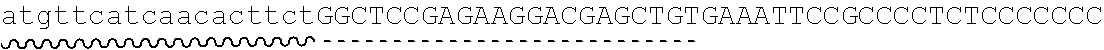

CCCCCTCTCCCTCCCCCCCCCCTAACGTTACTGGCCGAAGCCGCTTGGAATAAGG
CCGGTGTGCGTTTGTCTATATGTTATTTTCCACCATATTGCCGTCTTTTGGCAATG
TGAGGGCCCGGAAACCTGGCCCTGTCTTCTTGACGAGCATTCCTAGGGGTCTTTC
CCCTCTCGCCAAAGGAATGCAAGGTCTGTTGAATGTCGTGAAGGAAGCAGTTCCT
CTGGAAGCTTCTTGAAGACAAACAACGTCTGTAGCGACCCTTTGCAGGCAGCGG
AACCCCCCACCTGGCGACAGGTGCCTCTGCGGCCAAAAGCCACGTGTATAAGAT
ACACCTGCAAAGGCGGCACAACCCCAGTGCCACGTTGTGAGTTGGATAGTTGTG
GAAAGAGTCAAATGGCTCTCCTCAAGCGTATTCAACAAGGGGCTGAAGGATGCC
CAGAAGGTACCCCATTGTATGGGATCTGATCTGGGGCCTCGGTGCACATGCTTTA
CATGTGTTTAGTCGAGGTTAAAAAAACGTCTAGGCCCCCCGAACCACGGGGACG
TGGTTTTCCTTTGAAAAACACGATAACCGCCACCATGTCTCGCTCCGTGGCCCTT
GCTGTCCTCGCCTTACTCTCTCTTTCTGGCCTGGAGGCTATCCAGCgtgagtetctcctaccc
tcccgctctggtccttcctctcccgctctgcaccctctgtggccctcgctgtgctctctcgctccgtgacttcccttctccaagttctccttgg
tggcccgccgtggggctagtccagggctggatctcggggaagcggcggggtggcctoggagtggggaagggggtgcgcacccg
ggacgcgcgctacttgcccctttcggcggagcaggggagacctttggcctacggcgacgggagggtcgggacaaagtttaggg
cgtcgataagcgtcagagcgccgagottggggggggtttctcttccgctctttcgcggggcctctggctcccccagcgcagctgga
gtgggggacgggtaggctcgtcccaaaggcgcggcgctgaggtttgtgaacgcgtggaggggcgcttggggtctgggggaggcg
tcgcccgggtaagcctgtctgctgcggctctgcttcccttagactggagagctgtggacttcgtctaggcgcccgctaagttc

Any suitable promoter may be used for the knock-in of IL-15. In certain embodiments where the polynucleotide encoding IL-15 or a functional fragment thereof is integrated into the B2M locus, the promoter is the endogenous promoter of B2M. Thus, in some embodiments, the iPSC-NK cells comprise a knock-in of a polynucleotide encoding IL-15 or a functional fragment thereof into the B2M gene of the iPSC-NK cells, wherein the polynucleotide is operably linked to the native promoter of the B2M gene. In some embodiments, the polynucleotide encoding IL-15 or a functional fragment thereof is operably linked to the endogenous B2M promoter. Illustrative donor sequences for insertion of IL-15 into the B2M locus are shown in SEQ ID NOs: 25, 27, 28, 31-34, 37 and 38.

In some embodiments, the iPSC-NK cells comprise a knock-in of a polynucleotide encoding IL-15 or a functional fragment thereof into the TGFbR2 gene of the iPSC-NK cells, wherein the polynucleotide is operably linked to a constitutive promoter. In certain embodiments wherein the polynucleotide encoding IL-15 or a functional fragment thereof is integrated into the TGFbR2 locus, the promoter is the EF1α promoter (long version). In some embodiments, the iPSC-NK cells comprise a knock-in of polynucleotide encoding IL-15 or a functional fragment thereof into the TGFbR2 gene of the iPSC-NK cells, wherein the polynucleotide is operably linked to a EF1α promoter (long version). In some embodiments, the polynucleotide encoding IL-15 or a functional fragment thereof is operably linked to the endogenous TGFbR2 promoter. In some embodiments, the polynucleotide encoding IL-15 or a functional fragment thereof is operably linked to an EF1α promoter (e.g., EF1α promoter, long version). An illustrative sequence of the EF1α promoter (long version) is set forth in SEQ ID NO: 49. In some embodiments, the polynucleotide encoding IL-15 or a functional fragment thereof is operably linked to an EF1α promoter (e.g., EF1α promoter, short version). See Bae et al., J Biol Chem. 1995, 270 (49): 29460-29468 for a characterization of the TGFbR2 promoter region, and Zhen and Baum, Int. J. Med. Sci. 2014; 11 (5): 404-408 for a characterization of the EF1α promoters, each of which is incorporated by reference herein in its entirety for the sequences and types of promoters that may be used.

In some embodiments, the NK cells are genetically modified by deleting or inactivating (or “knocking out”) a gene encoding TGFβ receptor 2 (TGFbR2). Optionally, such iNK cells comprising a deletion in TGFbR2 further comprise a knock-in of IL-15 or a functional fragment thereof. Illustrative donor sequences for insertion of IL-15 into the TGFβR2 locus are shown in SEQ ID NO: 26, 29, 30, 35, and 36.

A gene may be inactivated, for example, by introducing a homozygous or heterozygous inactivating mutation into said gene. A homozygous inactivating mutation results in complete loss of protein function and, in some cases, loss of protein expression. In some embodiments, the NK cells provided herein are genetically modified by inactivating the TGFbR2 gene, e.g., by targeting an exon of TGFbR2. In certain embodiments, the TGFbR2 gene may be inactivated by introducing a polynucleotide encoding IL-15 or a functional fragment thereof into the TGFbR2 locus. In certain embodiments, the TGFbR2 gene may be inactivated by introducing a dominant negative form of TGFbR2 into the cell.

Exemplary sequences of gRNAs that may be used to knockout TGFbR2 in the iPSC-NK cells described herein are provided in Table 3. Nucleotides 1-20 of each of SEQ ID NOs: 6-12 are the gRNA sequence and nucleotides 20-23 of each of SEQ ID NOs: 6-12 are the protospacer adjacent motif (PAM).

TABLE 3
Exemplary gRNA Sequences for TGFBR2 knockout

| Name | Target | Sequence |
|---|---|---|
| TGFBR2 sgRNA#1 | Exon 1 | TGCTGGCGATACGCGTCCACAGG |
| | | (SEQ ID NO: 6) |
| TGFBR2 sgRNA#2 | Exon 1 | AACGTGCGGTGGGATCGTGCTGG |
| | | (SEQ ID NO: 7) |
| TGFBR2(_C1) sgRNA#3 | Exon 3 | ATGATAGTCACTGACAACAACGG |
| | | (SEQ ID NO: 8) |
| TGFBR2(_C2) sgRNA#4 | Exon 3 | AGTTGCTCATGCAGGATTTCTGG |
| | | (SEQ ID NO: 9) |
| TGFBR2(_C3) sgRNA#5 | Exon 3 | GAAGCCACAGGAAGTCTGTGTGG |
| | | (SEQ ID NO: 10) |
| TGFBR2 sgRNA#6 | Exon 1 | CCGACTTCTGAACGTGCGGTGGG |
| | | (SEQ ID NO: 11) |
| TGFBR2(_CN) sgRNA#7 | Exon 3 | TATCATGTCGTTATTAACTGAGG |
| | | (SEQ ID NO: 12) |

In some embodiments, the iPSC-NK cells provided herein are modified such that they are deficient in TGFbR2 signaling, e.g., by deleting the intracellular signaling domain of TGFbR2. This may be accomplished by, e.g., introducing a stop codon into a suitable position in the TGFbR2 amino acid sequence (for example, introducing a stop codon after the transmembrane domain). Exemplary gRNA sequences and their corresponding donor DNA sequences that may be used to knockout the TGFbR2 signaling domain by introducing a stop codon after the TGFbR2 transmembrane domain are provided in Table 4. Nucleotides 1-20 of each of SEQ ID NOs: 13 and 14 are the gRNA sequence and nucleotides 20-23 of each of SEQ ID NOs: 13 and 14 are the protospacer adjacent motif (PAM). Nucleotides 1-32 of each of SEQ ID NO: 15 and 16 are complementary to the sequence encoding the TGFbR2 transmembrane domain. Nucleotides 36-44 of each of SEQ ID NO: 15 and 16 encode three stop codons.

TABLE 4
gRNA and Donor DNA Sequences for knockout
of TGFBR2 signaling domain

| | | Sequence | Sequence of |
|---|---|---|---|
| Name | Target | of gRNA | Donor DNA |
| TGFBR2 | Exon 5 | TCTACTGCTACC | GAGTTGCCATATCT |
| sgRNA#7 | | GCGTTAACCGG | GTCATCATCATCTT |
| | | (SEQ ID NO: 13) | CTACTGCTGATGAT |
| | | | GATACCGCGTTAAC |
| | | | CGGCAGCAGAAGCT |
| | | | GAGTTCAACCTGGG |
| | | | AAAC |
| | | | (SEQ ID NO: 15) |
| TGFBR2 | Exon 5 | GCTTCTGCTGCC | GAGTTGCCATATCT |
| sgRNA#8 | | GGTTAACGCGG | GTCATCATCATCTT |
| | | (SEQ ID NO: 14) | CTACTGCTGATGAT |
| | | | GATACCGGGTTAAC |
| | | | CGGCAGCAGAAGCT |
| | | | GAGTTCAACCTGGG |
| | | | AAA |
| | | | (SEQ ID NO: 16) |

In certain preferred embodiments, the iPSC-NK cells comprise a homozygous inactivating mutation in the TGFβR2 gene and express detectable levels of IL-15 or a functional fragment thereof. In some embodiments, the IL-15 is mbIL-15.

In some embodiments, the method described herein (e.g., in this section) result in a population of iPSC-NK cells wherein about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90 to about 95%, or about 95% to 100% of cells comprise an inactivating mutation in TGFβR2 and/or express detectable levels of IL-15 or a functional fragment thereof. In some embodiments, the method described herein (e.g., in this section) result in a population of iPSC-NK cells wherein at least 50%, at least 55%, at least 60%, at least 65%, at last 70%, at least 75%, at least 80%, at least 85%, at least 90%, at least 95%, at least 98% or more than 98% of cells comprise an inactivating mutation in TGFβR2 and/or express detectable levels of IL-15 or a functional fragment thereof. The expression level of IL-15 or a functional fragment thereof may be determined using any suitable method known in the art or described herein, including, for example, flow cytometry, Western Blotting, Enzyme Linked Immunosorbent Assays (ELISA), quantitative real-time PCR (qPCR) and RNA sequencing. In some embodiments, the iNK cells comprise an inactivating (e.g., a frameshift) mutation in TGFbR2 and express a truncated form of TGFbR2 protein. The inacticating mutation in TGFbR2 may be biallelic or monoallelic.

In some embodiments, the iNK cells are resistant to the suppressive effect of TGFb signaling. For example, the expression level of NKG2D, DNAM and/or NKp30 on the surface of an iNK cell may remain comparable after treatment with TGFb1 to the levels before treatment.

In some embodiments, the iNK cells are able to survive without the stimulation of exogenous cytokines. Cell survival may be determined by measuring cell counts or cell viability.

In some embodiments, the iNK cells show higher cell killing ability than unmodified NK cells. Cell killing ability may be determined by incubating the iNK (and unmodified control NK) cells with target cells and measuring the disappearance of the target cells using, e.g., a fluorescent marker. In some embodiments, the iNK cells comprising an IL-15 knock-in and a TGFbR2 knockout described herein kill target cells with an efficiency that is about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 2-3 times, about 3-4 times, about 4-5 times, about 5-6 times, about 6-7 times, about 7-8 times, about 8-9 times or about 9-10 times higher than that of NK cells not comprising an IL-15 knock-in or a TGFbR2 knockout. In some embodiments, the iNK cells comprising an IL-15 knock-in and a TGFbR2 knockout described herein kill target cells with an efficiency that is about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 2-3 times, about 3-4 times, about 4-5 times, about 5-6 times, about 6-7 times, about 7-8 times, about 8-9 times or about 9-10 times higher than that of NK cells not comprising an IL-15 knock-in and a TGFbR2 knockout.

In some embodiments, the iNK cells comprising an IL-15 knock-in and a TGFbR2 knockout described herein kill target cells with an efficiency that is about 10%, about 20%, about 30%, about 40%, about 50%, about 60%, about 70%, about 80%, about 90%, about 100%, about 3 times, about 4 times, about 5 times, about 6 times, about 7 times, about 8 times, about 9 times or about 10 times higher than that of NK cells not comprising an IL-15 knock-in or a TGFbR2 knockout. In some embodiments, the iNK cells comprising an IL-15 knock-in and a TGFbR2 knockout described herein kill target cells with an efficiency that is about 10%, about 20%, about 30%, about 40%, about 50%, about 60%, about 70%, about 80%, about 90%, about 100%, about 3 times, about 4 times, about 5 times, about 6 times, about 7 times, about 8 times, about 9 times or about 10 times higher than that of NK cells not comprising an IL-15 knock-in and a TGFbR2 knockout.

In some embodiments, the iNK cells described herein persist longer in the circulation after intravenous administration to a patient. In some embodiments, the iNK cells comprising an IL-15 knock-in and a TGFbR2 knockout described herein persist in the circulation of a subject about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 2-3 times, about 3-4 times, about 4-5 times, about 5-6 times, about 6-7 times, about 7-8 times, about 8-9 times or about 9-10 times longer than NK cells not comprising an IL-15 knock-in or a TGFbR2 knockout. In some embodiments, the iNK cells described herein persist longer in the circulation after intravenous administration to a patient. In some embodiments, the iNK cells comprising an IL-15 knock-in and a TGFbR2 knockout described herein persist in the circulation of a subject about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 2-3 times, about 3-4 times, about 4-5 times, about 5-6 times, about 6-7 times, about 7-8 times, about 8-9 times or about 9-10 times longer than NK cells not comprising an IL-15 knock-in and a TGFbR2 knockout.

In some embodiments, the iNK cells comprising an IL-15 knock-in and a TGFbR2 knockout described herein persist in the circulation of a subject about 10%, about 20%, about 30%, about 40%, about 50%, about 60%, about 70%, about 80%, about 90%, about 3 times, about 4 times, about 5 times, about 6 times, about 7 times, about 8 times, about 9 times or about 10 times longer than NK cells not comprising an IL-15 knock-in or a TGFbR2 knockout. In some embodiments, the iNK cells comprising an IL-15 knock-in and a TGFbR2 knockout described herein persist in the circulation of a subject about 10%, about 20%, about 30%, about 40%, about 50%, about 60%, about 70%, about 80%, about 90%, about 3 times, about 4 times, about 5 times, about 6 times, about 7 times, about 8 times, about 9 times or about 10 times longer than NK cells not comprising an IL-15 knock-in and a TGFbR2 knockout.

The tumor microenvironment has a suppressive effect on NK cell function and inhibits NK cell function e.g., via soluble factors (e.g., cytokines), hypoxic conditions and/or low nutrient levels. See, e.g., Melaiu et al., Front. Immunol. 10:3038. In some embodiments, the iNK cells described herein are resistant to the suppressive effects of the tumor microenvironment.

Expression levels of IL-15 and/or TGFbR2 in the iNK cells and unedited control NK cells may be determined using any suitable method known in the art. For example, ELISA or Western Blot may be used to detect expression levels of IL-15 and/or TGFbR2 protein, and qPCR may be used to detect expression levels of IL-15 and/or TGFbR2 mRNA.

In some embodiments, the average expression level of TGFbR2 in a population of iNK cells is about 10% to about 20%, about 20% to about 30%, about 30% to about 40%, about 40% to about 50%, about 50% to about 60%, about 60% to about 70%, about 70% to about 80%, about 80% to about 90%, about 90% to about 95% or more than 95% lower than the average expression level of TGFbR2 in a population of unedited NK cells. In some embodiments, the expression level of TGFbR2 in the INK cells is about 10%, about 20%, about 30%, about 40%, about 50%, about 60%, about 70%, about 80%, about 90%, or about 95% lower than the expression level of TGFβR2 in unedited NK cells.

In the case of NK cells differentiated from iPSC, the genetic modifications described herein may be introduced before the differentiation of the iPSCs into iPSC-NK cells (“INK cells”). Modified iPSC cells may be cryopreserved before differentiation into iNK cells.

### Pharmaceutical Compositions

Also provided herein are pharmaceutical compositions and formulations comprising immune cells (e.g., NK cells) and a pharmaceutically acceptable carrier. In some embodiments, the pharmaceutical composition comprises iNK cells described herein.

In some embodiments, a pharmaceutical composition comprises a dose ranging from about 1×10⁵ to about 5×10⁵ iNK cells, about 5×10⁵ to about 1×10⁶ iNK cells, about 1×10⁶ to about 5×10⁶ iNK cells, about 5×10⁶ iNK cells to about 1×10⁷ iNK cells, about 1×10⁷ to about 5×10⁷ iNK cells, about 5×10⁷ to 1×10⁸ iNK cells, about 1×10⁸ to about 5×10⁸ iNK cells, about 5×10⁸ to about 1×10⁹ iNK cells, about 1×10⁹ to about 5×10⁹ iNK cells, about 5×10⁹ to 1×10¹⁰ iNK cells, about 1×10¹⁰ to about 5×10¹⁰ iNK cells, about 5×10¹⁰ to about 1×10¹¹ iNK cell, about 1×10¹¹ to about 5×10¹¹ iNK cells, about 5×10¹¹ to 1×10¹² INK cells, about 1×10¹² to about 5×10¹² iNK cells, about 5×10¹² to about 1×10¹³ iNK cells.

In certain embodiments, the pharmaceutical composition comprises an iNK cell population that is substantially pure.” As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present.

Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species. In some embodiments, a pharmaceutical composition comprises a population of iNK cells wherein about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90 to about 95%, or about 95% to 100% of cells comprise an inactivating mutation in TGFbR2 and/or express detectable levels of IL-15 or a functional fragment thereof. In some embodiments, a pharmaceutical composition comprises a population of iPSC-NK cells wherein at least 50%, at least 55%, at least 60%, at least 65%, at last 70%, at least 75%, at least 80%, at least 85%, at least 90%, at least 95%, at least 98% or more than 98% of cells comprise an inactivating mutation in TGFβR2 and express detectable levels of IL-15 or a functional fragment thereof.

In some embodiments, a pharmaceutical composition comprises a population of iNK cells wherein about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90 to about 95%, or about 95% to 100% of cells express one or more NK cell marker such as CD56 or CD45.

In some embodiments, a pharmaceutical composition comprises a population of iNK cells wherein at least 50%, at least 55%, at least 60%, at least 65%, at last 70%, at least 75%, at least 80%, at least 85%, at least 90%, at least 95%, at least 98% or more than 98% of cells express one or more NK cell marker selected from the group consisting of such as CD56 or CD45.

In some embodiments, a pharmaceutical composition is cryopreserved. A composition comprising iNK cells provided herein may be cryopreserved for about 1-3 months, about 3-6 months, about 6-9 months, or about 9-12 months. A composition comprising iPSC-NK cells provided herein may be cryopreserved for more than 3 months, more than 6 months, more than 9 months, more than 12 months, more than 18 months, more than 2 years, or more than 3 years before thawing and use in a method described herein. A formulation comprising the iPSC-NK cells described herein may further comprise a cryoprotectant. In such embodiments wherein the iPSC-NK cells are cryopreserved and thawed before use in a method described herein, the viability of the iPSC-NK cells is at least 30%, at least 50%, or at least 70% as determined by a suitable assay known in the art of described herein. Viability may be determined, e.g., using trypan blue exclusion.

Pharmaceutical compositions and formulations as described herein can be prepared by mixing the active ingredients (such as iPSC-NK cells) having the desired degree of purity with one or more optional pharmaceutically acceptable carriers (Remington's Pharmaceutical Sciences 22^{nd} edition, 2012).

The NK cells described herein can be incorporated into any pharmaceutical composition suitable for administration. Such compositions typically comprise the NK cells and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration, e.g., intravenous, administration. Solutions or suspensions used for intravenous administration can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above.

It is especially advantageous to formulate compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration. In some embodiments, the iPSC-NK cells described herein are diluted in normal saline before administration.

The formulation can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The formulations to be used for in vivo administration must be sterile. This is readily accomplished by filtration through sterile filtration membranes.

### Methods of Treatment

Further provided herein are methods of treating a medical disease or disorder in a subject by administering to the subject a therapeutically effective amount of NK cells provided herein. Therapeutic formulations of the invention, which comprise the iPSC-NK cells of the invention, are used to treat or alleviate a symptom associated with a cancer, including solid cancers and hematological cancers. Examples of cancers that may be treated with a method described herein include, without limitation, leukemias, lymphomas, breast cancer, colon cancer, ovarian cancer, bladder cancer, prostate cancer, glioma, lung & bronchial cancer, colorectal cancer, pancreatic cancer, esophageal cancer, liver cancer, urinary bladder cancer, kidney and renal pelvis cancer, oral cavity & pharynx cancer, uterine corpus cancer, and/or melanoma.

The present invention also provides methods of inhibiting the proliferation of tumor cells in a subject, comprising administering to the subject a population of iPSC-NK cells provided herein. A therapeutic regimen is carried out by identifying a subject, e.g., a human patient suffering from (or at risk of developing) a cancer, using standard methods.

Efficaciousness of treatment is determined in association with any known method for diagnosing or treating the particular immune-related disorder. Alleviation of one or more symptoms of the disease or disorder indicates that the treatment confers a clinical benefit. In some embodiments, a method provided herein results in decreased tumor proliferation in the subject.

As used herein, the “administration” of an agent, (e.g., a population of iPSC-NK cells), to a subject or subject includes any route of introducing or delivering to a subject a compound to perform its intended function. In a preferred embodiment, the population of iPSC-NK cells is administered intravenously. In some embodiments, the population of iPSC-NK cells provided herein is administered by intravenous infusion, e.g., an intravenous infusion over about 15 min, about 30 min, about 45 min, about 60 min, about 90 min, about 2 hours, about 3 hours, about 4 hours, or about 5 hours, or an intravenous infusion over about 15 min to about 30 min, about 30 min to about 45 min, about 45 min to about 60 min, about 60 min to about 90 min, about 90 min to about 2 hours, about 2 hours to about 3 hours, about 3 hours to about 4 hours, or about 4 hours to about 5 hours. The rate of infusion may vary with the number of cells being infused to the subject.

A therapeutically effective amount of the iPSC-NK cells of the invention relates generally to the amount needed to achieve a therapeutic objective. It is also to be appreciated that the various modes of treatment or prevention of medical conditions as described are intended to mean “substantial”, which includes total but also less than total treatment or prevention, and wherein some biologically or medically relevant result is achieved. In some embodiments, about 1×10⁵ to about 5×10⁵ INK cells, about 5×10⁵ to about 1×10⁶ iNK cells, about 1×10⁶ to about 5×10⁶ iNK cells, about 5×10⁶ iNK cells to about 1×10⁷ INK cells, about 1×10⁷ to about 5×10⁷ iNK cells, about 5×10⁷ to 1×10⁸ iNK cells, about 1×10⁸ to about 5×10⁸ iNK cells, about 5×10⁸ to about 1×10⁹ INK cells, about 1×10⁹ to about 5×10⁹ INK cells, about 5×10⁹ to 1×10¹⁰ INK cells, about 1×10¹⁰ to about 5×10¹⁰ iNK cells, about 5×10¹⁰ to about 1×10¹¹ iNK cell, about 1×10¹¹ to about 5×10¹¹ INK cells, about 5×10¹¹ to 1×10¹² iNK cells, about 1×10¹² to about 5×10¹² iNK cells, about 5×10¹² to about 1×10¹³ iNK cells are administered to the subject.

In some embodiments, one or more doses of the iPSC-NK cells are administered. If two or more doses of the iPSC-NK cells are administered, the duration between the administrations should be sufficient to allow time for propagation of the cells in the individual. In specific embodiments the duration between doses is 1, 2, 3, 4, 5, 6, 7, 8, 9, 10, 11, or 12 or more weeks.

As used herein, “treating” or “treatment” of a disease in a subject refers to (1) inhibiting the disease or arresting its development; or (2) ameliorating or causing regression of the disease or the symptoms of the disease. As understood in the art, “treatment” is an approach for obtaining beneficial or desired results, including clinical results. For the purposes of this disclosure, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

In some embodiments, a medical disease or disorder is treated by transfer of an immune cell population that elicits an immune response. In certain embodiments of the present disclosure, cancer or infection is treated by transfer of an immune cell population that elicits an immune response. Provided herein are methods for treating or delaying progression of cancer in an individual comprising administering to the individual an effective amount of iPSC-NK cells described herein. The present methods may be applied for the treatment of immune disorders, solid cancers, hematologic cancers, and viral infections.

The terms “cancer,” “neoplasm,” and “tumor,” used interchangeably and in either the singular or plural form, refer to cells that have undergone a malignant transformation that makes them pathological to the host organism. Non-limiting examples of cancers that may be treated according to the methods of the present disclosure include hematological malignancies and solid tumors. Non-limiting examples of solid tumors include hepatocellular carcinoma.

Tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma: leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; bepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma: oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); myelodysplastic syndrome (MDS); chronic myeloblasts leukemia; diffuse large B-cell lymphoma (DLBCL); peripheral T-cell lymphoma (PTCL); or anaplastic large cell lymphoma (ALCL). In some embodiments, the cancer is hepatocellular carcinoma.

In certain embodiments of the present disclosure, iPSC-NK cells are delivered to an individual in need thereof, and the individual has been diagnosed with a cancer. Without wishing to be bound by theory, the cells then enhance the individual's immune system to attack or directly attack the respective cancer or pathogenic cells.

### Combination Therapies

The iPSC-NK cells described herein may be administered in combination with one or more other therapeutic agents.

In some embodiments, the additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the subject can be administered nonmyeloablative lymphodepleting chemotherapy prior to the immune cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine. An exemplary route of administering cyclophosphamide and fludarabine is intravenously. Likewise, any suitable dose of cyclophosphamide and fludarabine can be administered. In particular aspects, around 60 mg/kg of cyclophosphamide is administered for two days after which around 25 mg/m² fludarabine is administered for five days.

In some embodiments, the subject can be administered nonmyeloablative lymphodepleting immunotherapy prior to the immune cell therapy. The nonmyeloablative lymphodepleting immunotherapy can be any suitable such therapy, which can be administered by any suitable route. The nonmyeloablative lymphodepleting immunotherapy can comprise, for example, the administration of an anti-CD52 agent or anti-CD20 agent. In some embodiments, the lymphodepleting immunotherapy is an anti-CD52 antibody. In some embodiments, the anti-CD52 antibody is alemtuzumab. In some embodiments, the lymphodepleting immunotherapy is an anti-CD20 antibody. Exemplary anti-CD20 antibodies include, but are not limited to rituximab, ofatumumab, ocrelizumab, obinutuzumab, ibritumomab or iodine i131 tositumomab. An exemplary route of administering anti-CD52 agent or anti-CD20 agent is intravenously. Likewise, any suitable dose of anti-CD52 agent or anti-agent can be administered.

In certain embodiments, a growth factor that promotes the growth and activation of the immune cells is administered to the subject either concomitantly with the immune cells or subsequently to the immune cells. The immune cell growth factor can be any suitable growth factor that promotes the growth and activation of the immune cells. Examples of suitable immune cell growth factors include interleukin (IL)-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2. In some embodiments, the iPSC-NK cells are not administered in combination with an interleukin.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

Combination therapies can include, but are not limited to, one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetyls alicylic acid, ibuprofen or naproxen sodium), cytokine antagonists (for example, anti-TNF and anti-IL-6), cytokines (for example, interleukin-10 or transforming growth factor-beta), hormones (for example, estrogen), or a vaccine. In addition, immunosuppressive or tolerogenic agents including but not limited to calcineurin inhibitors (e.g., cyclosporin and tacrolimus); mTOR inhibitors (e.g., Rapamycin); mycophenolate mofetil, antibodies (e.g., recognizing CD3, CD4, CD40, CD154, CD45, IVIG, or B cells); chemotherapeutic agents (e.g., Methotrexate, Treosulfan, Busulfan); irradiation; or chemokines, interleukins or their inhibitors (e.g., BAFF, IL-2, anti-IL-2R, IL-4, JAK kinase inhibitors) can be administered. Such additional pharmaceutical agents can be administered before, during, or after administration of the immune cells, depending on the desired effect. This administration of the cells and the agent can be by the same route or by different routes, and either at the same site or at a different site.

The iPSC-NK cells may be administered before, during, or after, an additional therapeutic agent, such as an immune checkpoint inhibitor. The administrations may be in intervals ranging from concurrently to minutes to days to weeks. In embodiments where the iPSC-NK cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the iPSC-NK cell therapy and the anti-cancer therapy (e.g., an immune checkpoint inhibitor) within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

### Chemotherapy

A wide variety of chemotherapeutic agents may be used in accordance with the iPSC-NK cells provided herein. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin: CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin: spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, decitabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine: trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein transferase inhibitors, transplatinum, and pharmaceutically acceptable salts, acids, or derivatives of any of the above. In some embodiments, azacitidine is administered at 75 mgs/m² subcutaneously.

### Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### Immunotherapy

The skilled artisan will understand that immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells.

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al, 1998); cytokine therapy, e.g., interferons α, β, and γ, IL-1, GM-CSF, and TNF (Bukowski et al, 1998; Davidson et al, 1998; Hellstrand et al, 1998); gene therapy, e.g., TNF, IL-1, IL-2, and p53 (Qin et al, 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al, 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12 (4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadbesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD 152. The complete cDNA sequence of human CTLA-4 has the Genbank accession number L15006. CTLA-4 is found on the surface of T cells and acts as an “off switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95 (17): 10067-10071; Camacho et al. (2004)/Clin Oncology 22 (145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10DI, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

Examples of immunotherapies for use in treatment of kidney cancer or renal cell cancer include, but are not limited to Afinitor (Everolimus), Afinitor Disperz (Everolimus), Aldesleukin, Avastin (Bevacizumab), Avelumab, Axitinib, Bavencio (Avelumab), Bevacizumab, Cabometyx (Cabozantinib-S-Malate), Cabozantinib-S-Malate, Everolimus, IL-2 (Aldesleukin), Inlyta (Axitinib), Interleukin-2 (Aldesleukin), Ipilimumab, Keytruda (Pembrolizumab), Lenvatinib Mesylate, Lenvima (Lenvatinib Mesylate), Mvasi (Bevacizumab), Nexavar (Sorafenib Tosylate), Nivolumab, Opdivo (Nivolumab), Pazopanib, Hydrochloride, Pembrolizumab, Proleukin (Aldesleukin), Sorafenib Tosylate, Sunitinib Malate, Sutent (Sunitinib Malate), Temsirolimus, Torisel (Temsirolimus), Votrient (Pazopanib Hydrochloride), Yervoy (Ipilimumab).

Examples of immunotherapies for use in treatment of Acute Myeloid Leukemia (AML) include, but are not limited to Azacitidine, Arsenic Trioxide, Cerubidine (Daunorubicin Hydrochloride), Cyclophosphamide, Cytarabine, Daunorubicin Hydrochloride, Daunorubicin Hydrochloride and Cytarabine Liposome, Daurismo (Glasdegib Maleate), Dexamethasone, Doxorubicin Hydrochloride, Enasidenib Mesylate, Gemtuzumab Ozogamicin, Gilteritinib Fumarate, Glasdegib Maleate, Idamycin PFS (Idarubicin Hydrochloride), Idarubicin Hydrochloride, Idhifa (Enasidenib Mesylate), Ivosidenib, Midostaurin, Mitoxantrone Hydrochloride, Mylotarg (Gemtuzumab Ozogamicin), Rubidomycin (Daunorubicin Hydrochloride), Rydapt (Midostaurin), Tabloid (Thioguanine), Thioguanine, Tibsovo (Ivosidenib), Trisenox (Arsenic Trioxide), Venclexta (Venetoclax), Venetoclax, Vincristine Sulfate, Vyxeos (Daunorubicin Hydrochloride and Cytarabine Liposome), Xospata (Gilteritinib Fumarate).

In some embodiments, a patient treated in accordance with a method described herein may be administered an NK cell engager, for example, an NKp46 engager.

### Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### Articles of Manufacture or Kits

An article of manufacture or a kit is provided comprising the iPSC-NK cells is also provided herein. The article of manufacture or kit can further comprise a package insert comprising instructions for using the iPSC-NK to treat or delay progression of cancer in an individual or to enhance immune function of an individual having cancer. Suitable containers include, for example, bottles, vials, bags and syringes. The container may be formed from a variety of materials such as glass, plastic (such as polyvinyl chloride or poly olefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### EXAMPLES

### Example 1: Generation of IL-15 Knock-In and TGFbR2 Knockout iPSC

### TALEN Gene-Editing Procedures

Gene editing strategies are outlined in FIGS. 2A and 2B. FIG. 2A shows the non-destructive IL-15 knock-in (KI) strategy and FIG. 2B shows the EF1α-IL-15-bGHpA disruptive KI strategy, which results in knock in of IL-15 and knock out of TGFbR2. Gene edits were generated with TALEN by electroporation using LONZA 4D nucleofector X, The electroporated iPSC cells were cultured in the E8 medium for 3-5 days before dissociation by Tryp-LE and resuspension in PBS. Using a cell sorting machine, single iPSC cells were sorted into single wells of a 96-well plate. After culturing in the 96-well plate for 8-10 days, the single clone was transferred to duplicate wells of 24-well plate. Then genomic DNA of single clone was extracted and purified. Each clone was identified by PCR with specific primers to distinguish wildtype, monoallelic knock-in or biallelic knock-in. The amplified band from the positive clone was sent for Sanger sequencing or Amplicon sequencing.

To knock in IL-15 at the non-disruptive B2M locus, non-edited iPSC cells were electroporated with mRNAs encoding a B2M-targeted TALEN pair and then with an IL-15 donor DNA template (“LHA-IL-15-P2A-RHA donor oligo”, SEQ ID NO: 25). Clones #231 and #242 were identified as having a true IL-15 knock in at non-disrupted B2M locus (see “Sequence Verification” below) and selected for further experiments.

To generate TGFbR2 knockout cells, non-edited iPSC cells were electroporated with mRNAs encoding a TGFbR2-targeted TALEN pair.

To generate the double edits (iPSCs comprising both an IL-15 knock in and a TGFbR2 knockout), two approaches were used: The first approach was electroporating non-edited iPSCs with both the mRNAs encoding a TGFbR2-targeted TALEN pair and an IL-15 donor plasmid (“RHA-EF1α-IL15-bGHpA-LHA”, SEQ ID NO: 26). From this approach, clone #318 was identified as a true double edited clone with both alleles of TGFbR2 knocked out and a biallelic IL-15 KI at the disruptive TGFbR2 locus.

The second approach was to electroporate the iPSC cells from clone #231 or #242 (the true IL-15 knock in clones described above) with mRNAs encoding a TGFbR2-targeted TALEN pair. From this approach, clones #331, #337, #338, and #341 were identified as the true double edited clones with both TGFbR2 alleles knocked out and a biallelic IL-15 KI at the non-disruptive B2M locus. Besides these clones, a number of clones with monoallelic KI and/or KO were also obtained.

Sequencing Verification of the KI and/or KO iPSC Clones

The IL-15 KI and TGFbR2 KO in the iPSC clones were verified using sequencing.

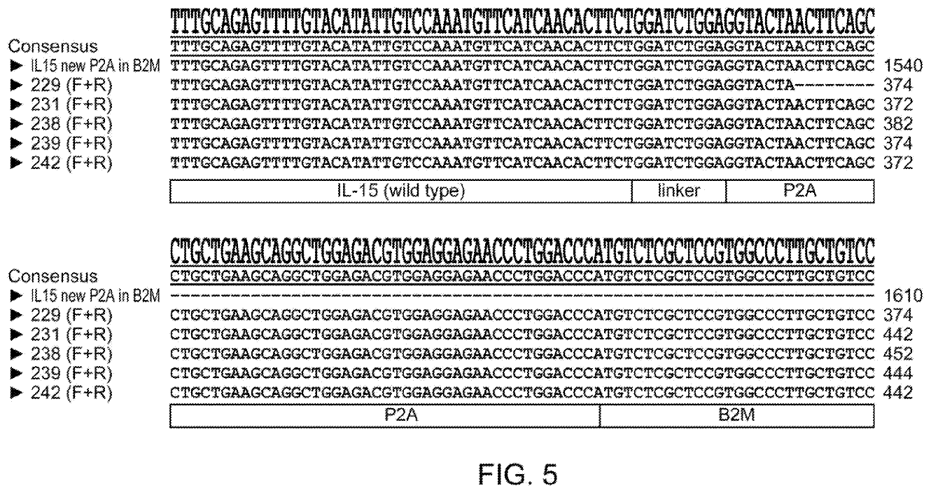

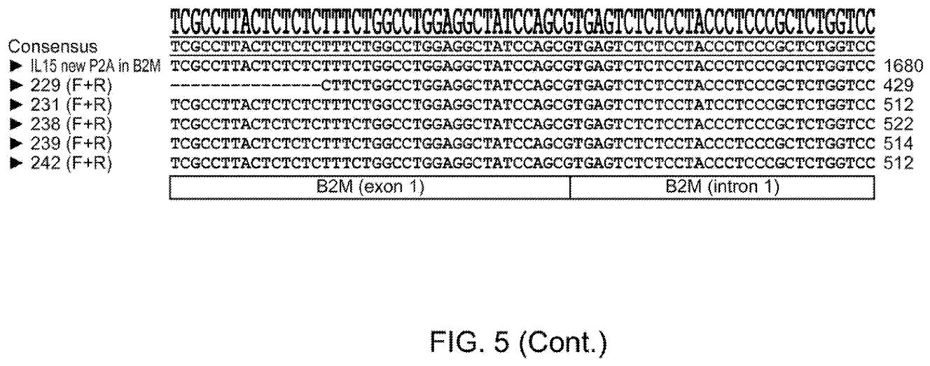

FIG. 5 shows sequencing results of the IL-15-P2A-B2M joint area of the biallelic IL-15 knock-in iPSC single clones. Clones #231, #238, #239, and #242 showed the expected sequencing results and were identified as true non-disruptive IL-15 KI clones. A 93 base pair deletion was identified in clone #229, and a heterozygous mutation in intron 1 if B2M was identified in the #231 colony, and therefore clones #229 and #231 were not considered true non-disruptive IL-15 KI clones.

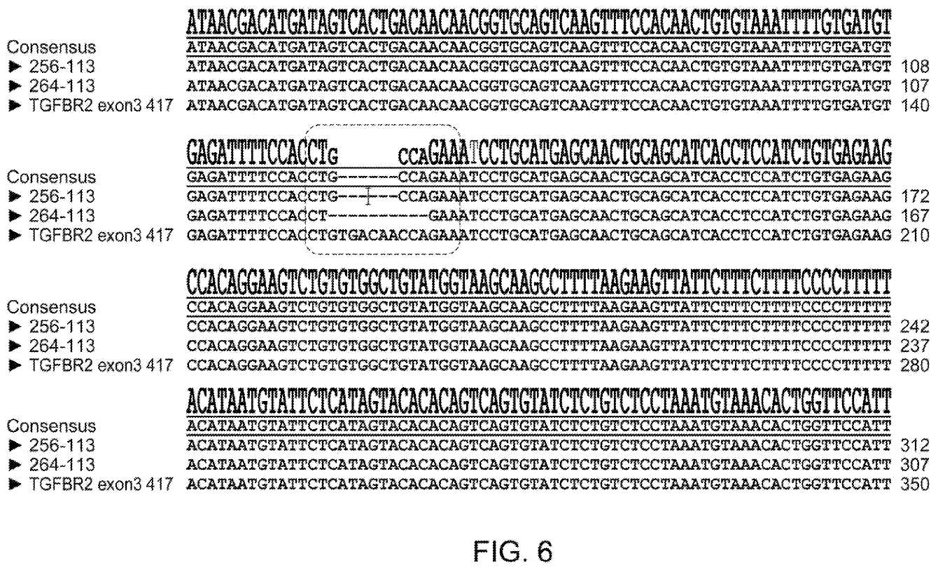

FIG. 6 shows sequencing results of the TGFbR2 locus in TGFbR2 KO iPSC cells (clones #255, #256, and #269). All three clones showed some deletion in the TGFbR2 locus. However, clone #256 had a 6 bp deletion which did not induce a frame-shift, and was not selected for further experiments. Clone #264 had a 10 bp deletion which did induce a frame-shift and was considered a true TGFbR2 KO clone, but showed slow growth in further experiments. Clone #269 was a true TGFbR2 KO clone with a heterozygous TGFbR2 KO (10 bp deletion and 13 bp deletion).

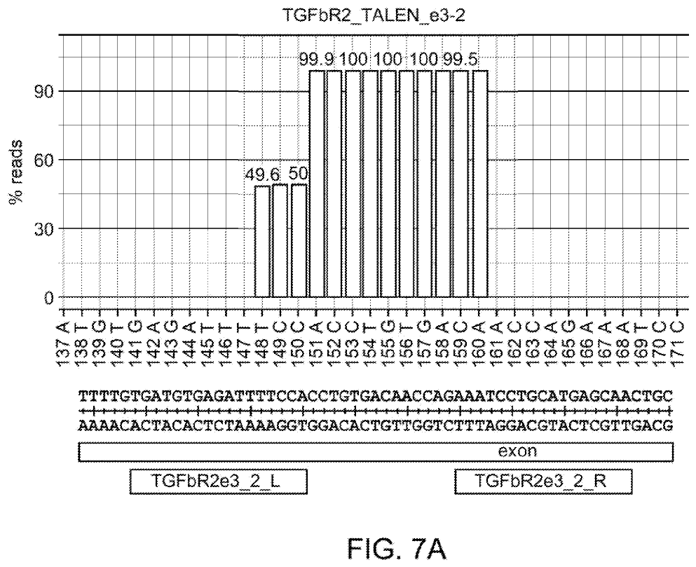

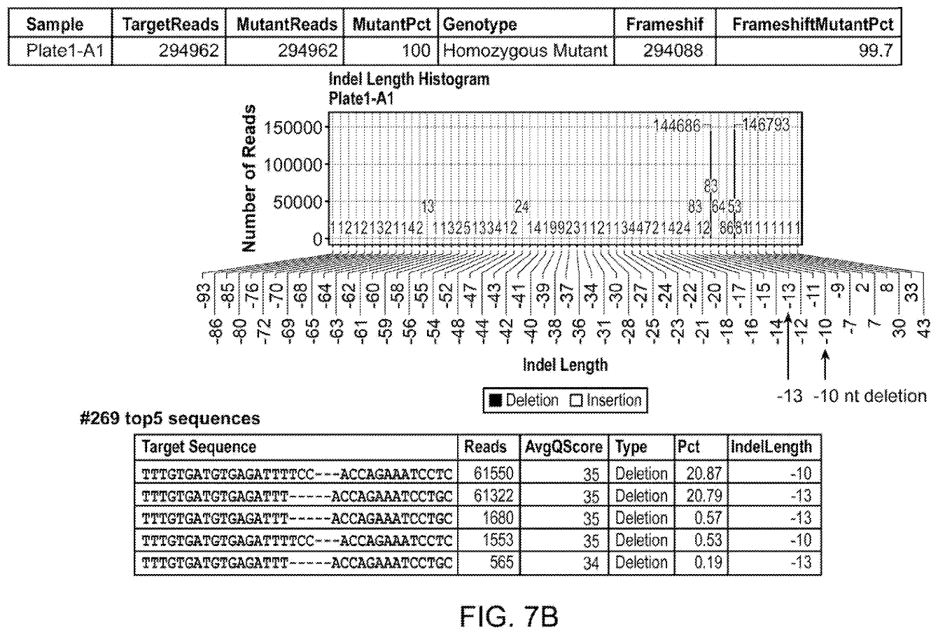

Clone #269 was further studied using amplicon sequencing to determine the genetic variation in the clones. Results shown in FIGS. 7A and 7B indicate that clone #269 is a TGFbR2 10 nt and 13 nt biallelic heterozygous clone. There was a 10 bp deletion on one allele, and a 13 bp deletion on the other allele. Both deletions induced frame-shifting. FIG. 7A is a graph to show the reading % on TGFbR2 locus. FIG. 7B is a detailed analysis of those readings. The reading % data suggested that clone #269 comes from a single cell because each deletion accounts ˜50% of reads. This true TGFbR2 KO was also verified by western blot (see below).

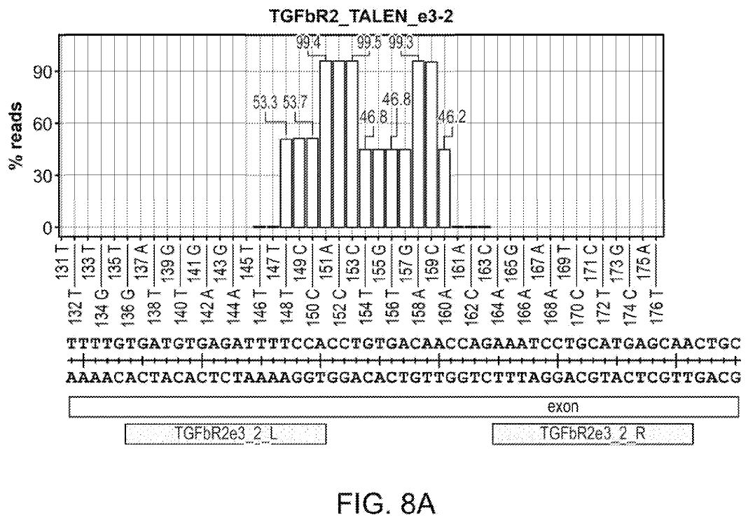

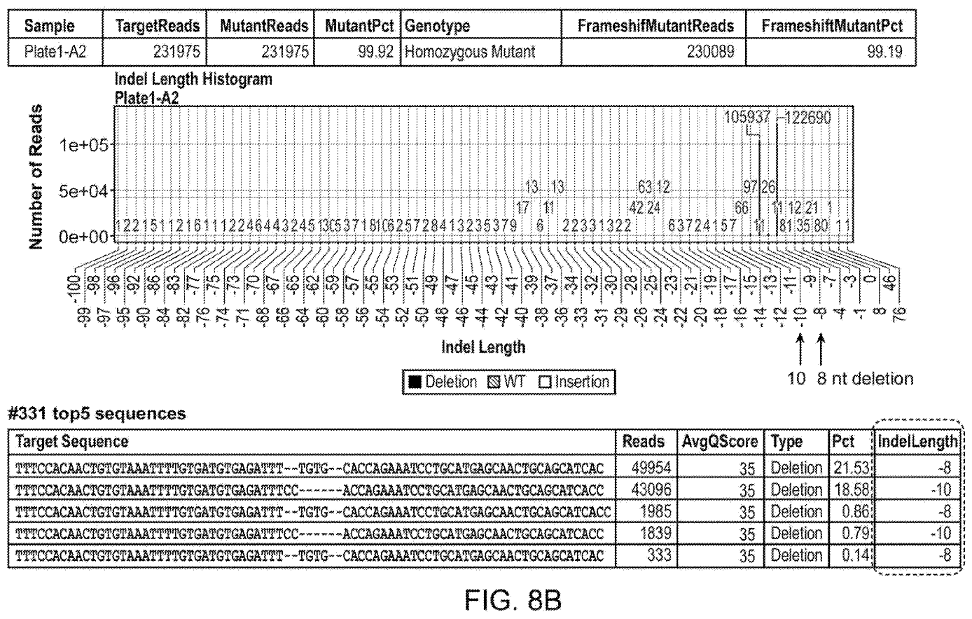

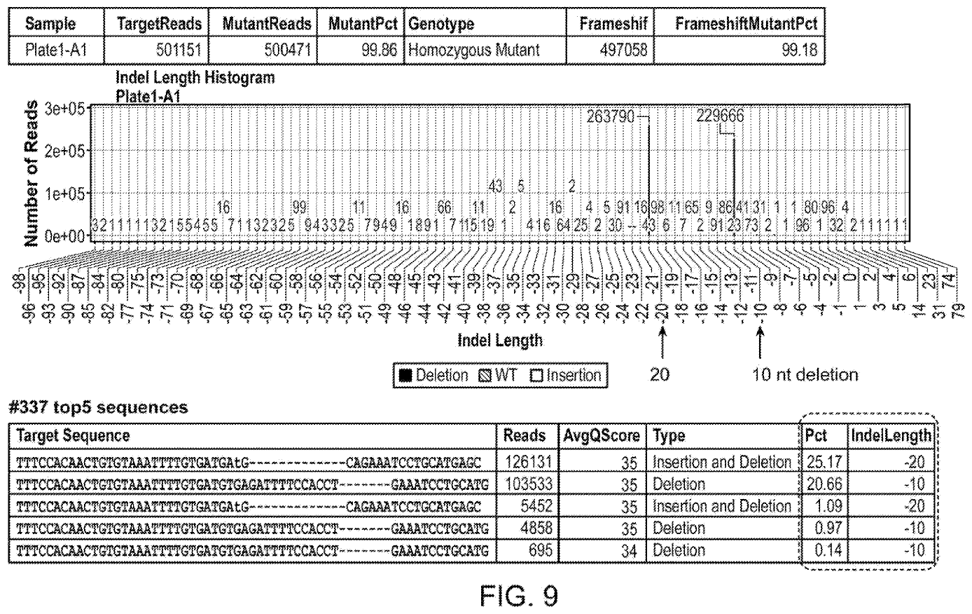

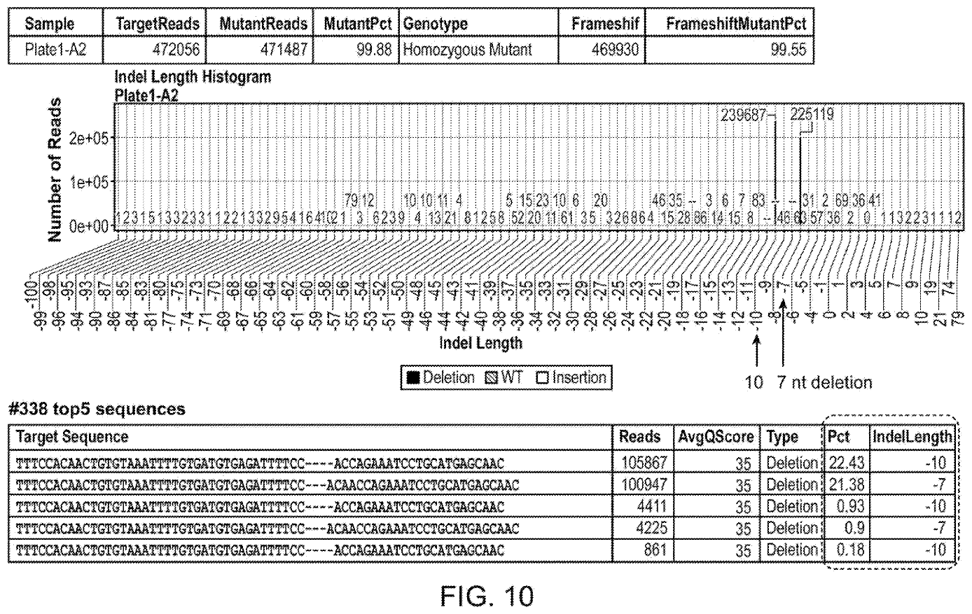

Clones #331, #337, and #338 (the true double edited clones with both TGFbR2 alleles knocked out and a biallelic IL-15 KI at the non-disruptive B2M locus identified above) were further analyzed using amplicon sequencing. Results shown in FIGS. 8A and 8B indicate that clone #331 is a TGFbR2 10 nt and 8 nt biallelic heterozygous clone. There was a 10 bp deletion on one allele, and an 8 bp deletion on the other allele. Both deletions induced frame-shifting. FIG. 8A is a graph to show the reading % on TGFbR2 locus. FIG. 8B is a detailed analysis of those readings. The reading % data, suggested that clone #331 comes from a single cell because the deletion on each allele accounts for ˜50% of reads. This true TGFbR2 KO was also verified by western blot (see below). Results shown in FIG. 9 indicate that clone #337 is a TGFbR2 20 nt and 10 nt biallelic heterozygous clone. Results shown in FIG. 10 indicate that clone #338 is a TGFbR2 10 nt and 7 nt deletion biallelic, heterozygous clone.

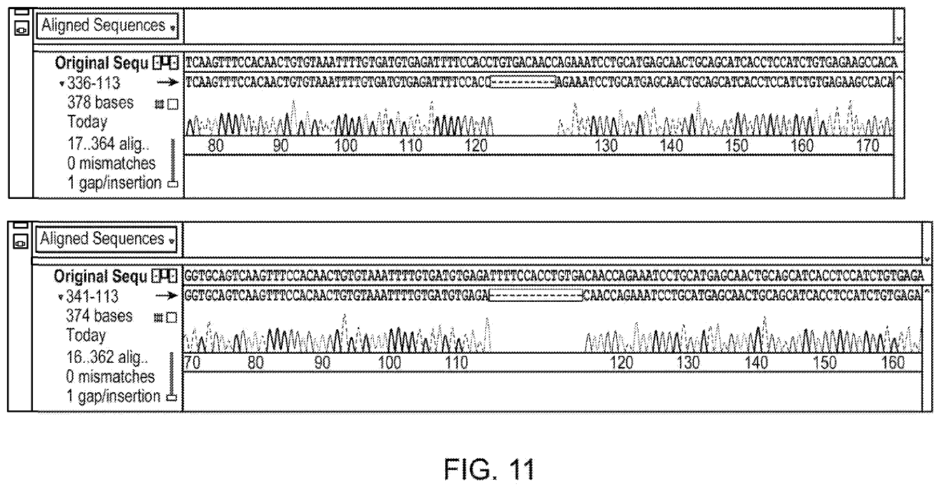

Clones #336 and #341, generated by sequential IL-15 KI (yielding clones #231 and #242) followed by TGFbR2 KO as described above, were verified by Sanger sequencing. Similarly, clone (#318), generated by one-time gene editing of IL-15 KI into the TGFbR2 locus was verified by Sanger sequencing. Results shown in FIG. 11 indicate that clone #336 has a 10 nt homozygous deletion in TGFbR2 and that clone #341 has a 14 nt homozygous deletion in TGFbR2. The accurate gene edits for clone #318 were also confirmed by sequencing.

Target Protein Expression Level Analysis of the Edited iPSC Clones

Expression levels of IL-15 protein were analyzed by ELISA. The iPSC cells were cultured in E8 medium The medium (supernatant) was collected after three days, when the iPSC cells' confluency was over 80%. A standard curve was prepared using 50 μL diluted human IL-15 protein. To avoid missing weak signals, the sample volume in the reaction was tripled. Expression levels of TGFbR2 protein in whole-cell extracts were analyzed by western blot using a recombinant anti-TGFbR2 antibody (Abcam catalogue number ab184948). The expected band size was 65 kDa and the observed band size was 80 kDa.

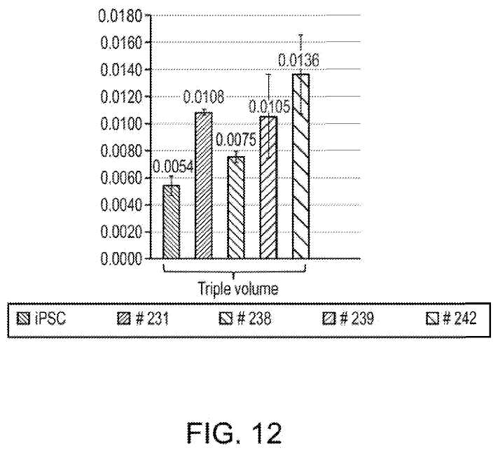

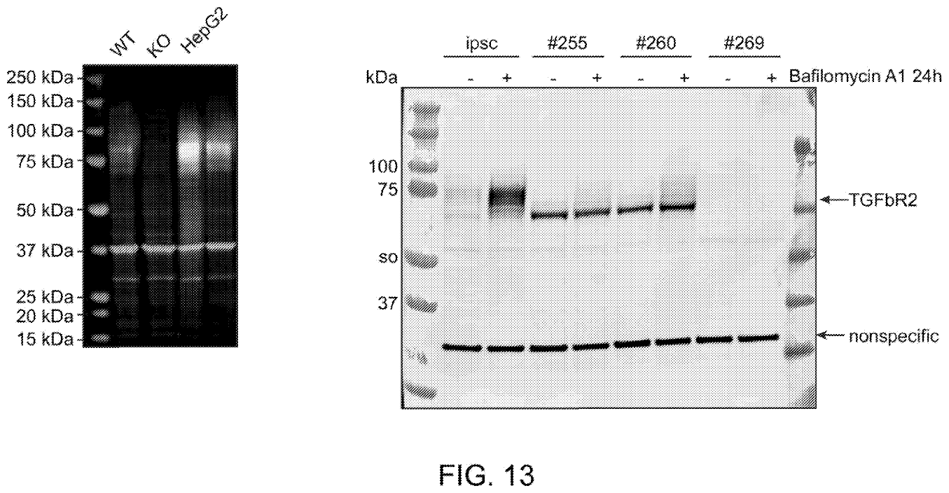

The secreted IL15 was detected by ELISA in the medium of the bialllic IL-15 KI iPSC colonies (clones #231, #238, #239, and #242), but not in controls (non-edited iPSCs) (FIG. 12). The TGFbR2 protein was detected in unedited iPSCs, and in clones #255 and #260, but not clone #269, indicating that out of these three colonies only clone #269 is a true TGFbR2 KO clone (FIG. 12). Sequencing analysis showed that clone #260 had a 10 bp deletion on one allele, and 6 bp deletion (no frame-shifting) on another allele. Therefore, the truncated TGFbR2 protein could be detected in the sample of #260.

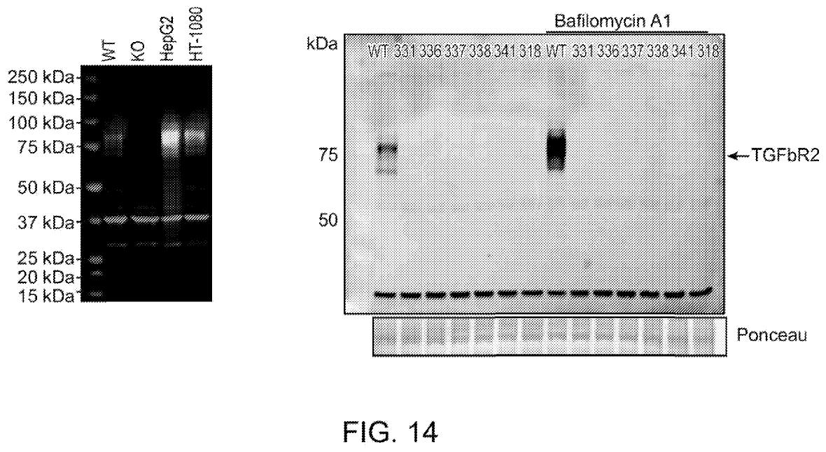

The TGFbR2 protein could not be detected by western blot in clones #331, #336, #337, #338, #341, and #318, even with Bafilomycin A1 treatment, while it could be detected in non-edited iPSCs with or without Bafilomycin A1 treatment (FIG. 14). Bafilomycin A1 increases TGFbR2 expression and was used here to enhance the signal. Thus, several of the true TGFbR2 KO and IL-15 KI clones were analyzed by western blot with or without Bafilomycin A1 for detection of TGFbR2 protein expression. These results are consistent with sequencing results.

### Example 2: Generation of INK Cells

The clones from Example 1 were used to generated iNK cells. Thus, iPSCs from these clones were first differentiated into hematopoietic progenitor cells (CD34+ cells) using spin EB methods with ROCKi. Then the EBs were transferred into NK cell differentiation conditions for 3-5 weeks followed by expansion with feeder cells for about 3 weeks to obtain pure and functional NK cells. Details of this method are described in Zhu, H., Kaufman, D. S. (2019). An Improved Method to Produce Clinical-Scale Natural Killer Cells from Human Pluripotent Stem Cells. In: Kaneko, S. (eds) In Vitro Differentiation of T-Cells. Methods in Molecular Biology, vol 2048. Humana, New York, NY. https://doi.org/10.1007/978-1-4939-9728-2_12,

The purity of the iNKs from each clone was determined by flow cytometry using CD56 expression as a marker of iNK cells. Results are shown in Table 5.

TABLE 5
Purity of iNKs

| | % of CD56+ cells |
|---|---|
| iNKs from clone #242 | 98.28 |
| iNKs from clone #269 | 96.94 |
| iNKs from clone #318 | 96.72 |
| iNKs from clone #341 | 96.85 |

### Target Protein Expression Level in Edited iNK Cells

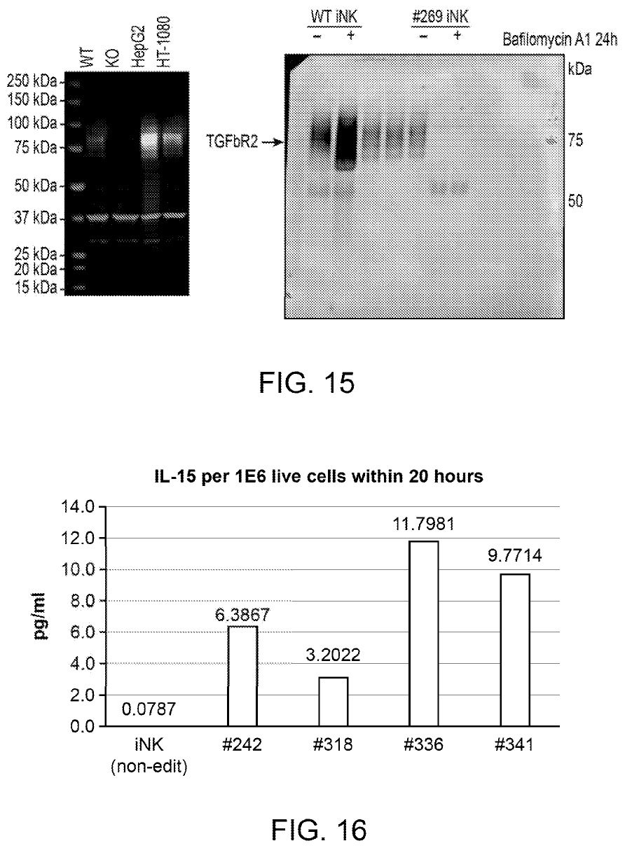

Expression levels of TGFbR2 protein were analyzed by Western blot using a recombinant anti-TGFbR2 antibody (Abcam catalogue number ab184948). The expected band size was 65 kDa and the observed band size was 80 kDa. TGFbR2 expression was detected in unedited (wiltype, WT) iNK cells but not in edited iNK cells differentiated from clone #269 (FIG. 15).

Levels of secreted IL-15 were determined by ELISA. IL-15 could not be detected in the medium of unedited iNKs, but was detected in the medium of iNKs differentiated from clones #242, #318, #336, or #341 (FIG. 16).

### Functional Characterization of Edited iNK Cells

The iNK cells generated above were further studied in functional assays.

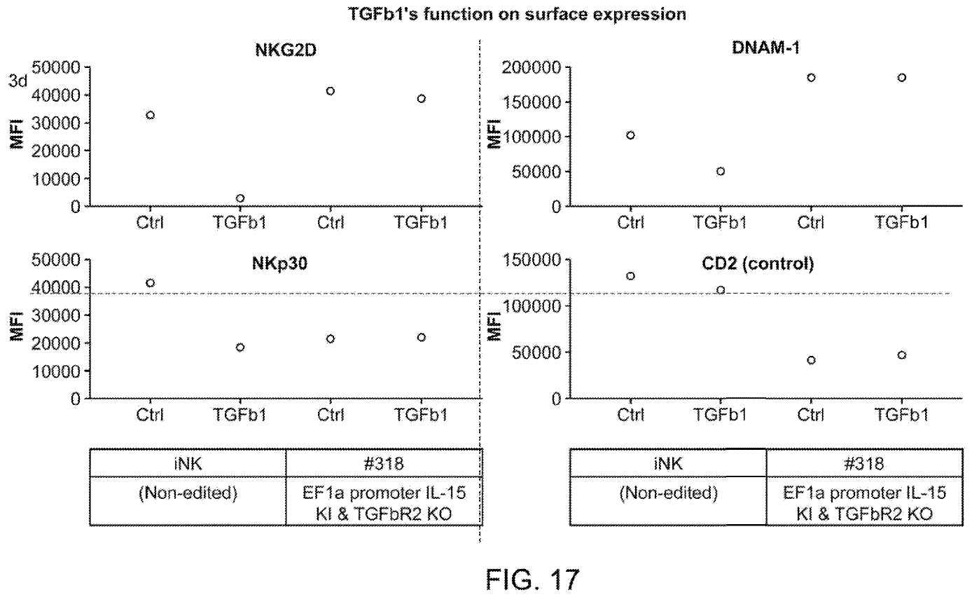

To determine the effect of TGFb signaling on iNK cells, iNK cells were treated with 10 ng/ml TGFb1 for 3 days and then harvested. The cells were stained with antibodies against NKG2D, NKp30, DNAM-1, and CD2, and analyzed by flow cytometry. Results shown in FIG. 17 indicate that the edited iNK cells were resistant to the suppressive TGFb signaling on NK cells.

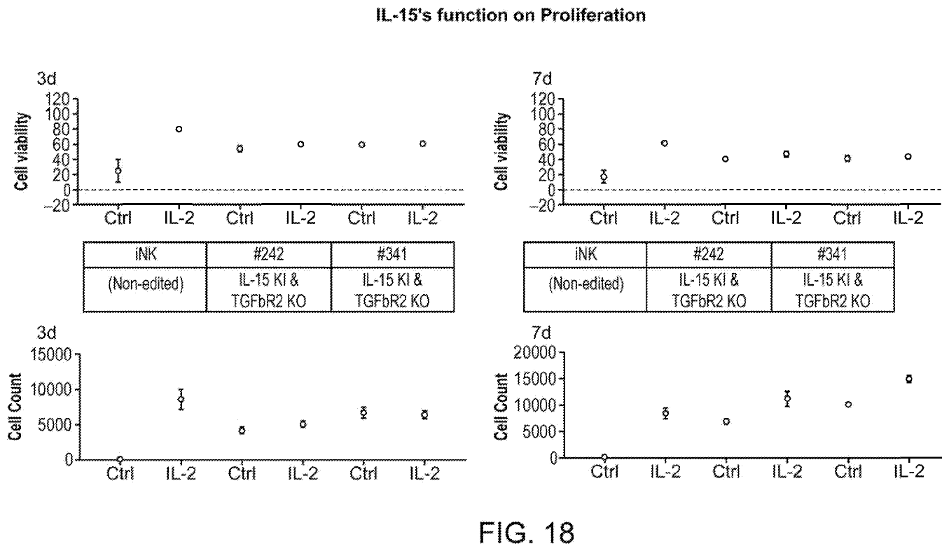

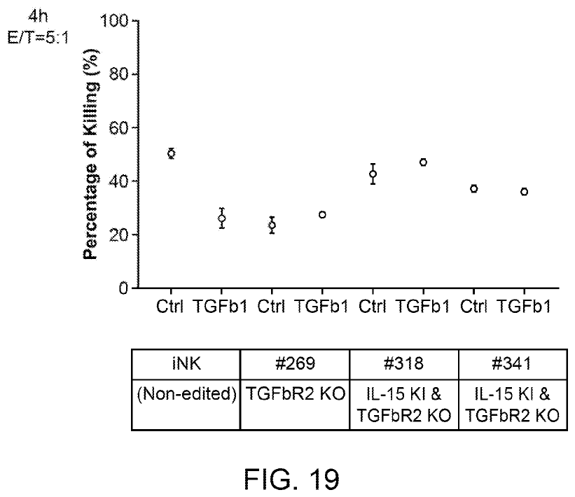

To determine the need of cytokines for iNK cell survival, iNK cells were cultured with or without IL-2 (500 units/ml) for 6 days. Cells were then harvested and stained with viability dye before flow cytometry analysis. Data shown in FIG. 18 indicates that the edited iNK cells could survive without endogenous cytokines.

The cell-killing ability of iNK cells was determined using flow cytometry. iNK cells were treated with 10 ng/ml TGFb1 for 3 days. Cells were then harvested and seeded with Hep3B-GFP target cells at an effector-to-target (ET) ratio of 5:1 for 4 hours. NK Cytotoxicity was performed by viability dye staining and flow cytometry detection.

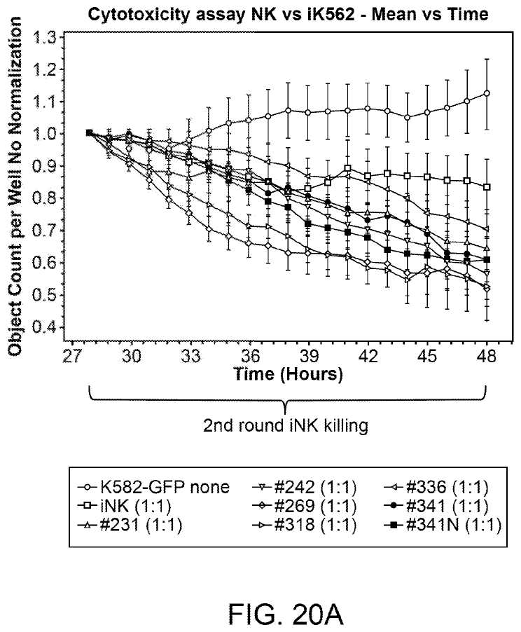

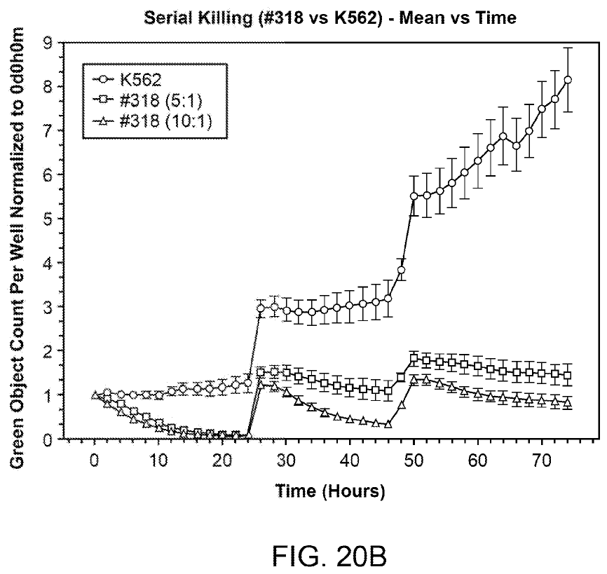

The serial cell killing ability of iNK cells was determined using an IncuCyte S3 instrument with real-time and automated analysis. Thus, 1×10⁴ control NK cells or 1×10⁴ edited iNK cells were co-cultured with 1×10⁴ K562 cells in the 96 well plate at an ET ratio of 1:1. K562 alone and non-edited control NK cells were used as controls. The edited iNKs show enhanced cytotoxicity effects (FIG. 20A). Serial cytotoxic activity of iNK cells against K562 tumor cells was similarly analyzed using an IncuCyte S3 instrument with real-time, automated analysis. The ET ratio was 5:1 or 10:1 (1×10⁵ edited iNK cells or 5×10⁴ edited iNK cells were co-cultured with 1×10⁴ K562 cells). After 24 hours and 48 hours, another 1×10⁴ K562 cells were added to the same well. K562 alone were used as a control. The edited iNKs could show serial cytotoxicity effects against tumor cells (FIG. 20B).

### INCORPORATION BY REFERENCE

All publications, patents, and Accession numbers mentioned herein are hereby incorporated by reference in their entirety as if each individual publication or patent was specifically and individually indicated to be incorporated by reference.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A cell population, comprising differentiated cells derived from induced pluripotent stem cells, said differentiated cells having a Natural Killer cell phenotype (INK cells), wherein the iNK cells comprise an inactivating mutation in the TGFbR2 gene, and wherein the iNK cells express interleukin-15 (IL-15) or a functional fragment thereof.

2. The cell population of claim 1, wherein the iNK cells comprise a homozygous inactivating mutation in the TGFbR2 gene.

3. The cell population of claim 1, wherein the iNK cells comprise a heterozygous inactivating mutation in the TGFbR2 gene.

4. The cell population of claim 1, wherein the iNK cells express a cell membrane-bound form of IL-15 (mbIL-15) or functional fragment thereof, or a cell membrane-bound form of IL-15 (mbIL-15) or functional fragment thereof fused to the IL-15 Receptor alpha (IL-15Rα), or IL-15 trapped in the endoplasmic reticulum (ER), or a soluble form of IL-15 or functional fragment thereof.

5. (canceled)

6. (canceled)

7. (canceled)

8. The cell population of claim 1, wherein the iNK cells comprise a knock-in of a polynucleotide encoding the IL-15 or functional fragment thereof into the B2M gene of the iPSC-NK cells or into the TGFbR2 gene of the iPSC-NK cells.

9. (canceled)

10. The cell population of claim 8, wherein the polynucleotide encoding the IL-15 or functional fragment thereof is operably linked to a promoter.

11. The cell population of claim 10, wherein the promoter is an exogenous promoter, a constitutive promoter, an EF1α promoter (short version) or an EF1α promoter (long version), or an endogenous promoter.

12. (canceled)

13. (canceled)

14. (canceled)

15. The cell population of claim 1, wherein the expression level of TGFbR2 in the iNK cells is about 10%, about 20%, about 30%, about 40%, about 50%, about 60%, about 70%, about 80%, about 90%, or about 95% lower than the expression level of TGFbR2 in a population of unedited NK cells.

16. The cell population of claim 1, wherein at least 50%, at least 55%, at least 60%, at least 65%, or at last 70%, of cells express one or more NK cell marker selected from the group consisting of CD56 and CD45.

17. A method of producing a cell population comprising differentiated cells derived from induced pluripotent stem cells, said differentiated cells having a Natural Killer cell phenotype (iNK cells), wherein the iNK cells comprise an inactivating mutation in the TGFbR2 gene, and wherein the iNK cells express interleukin-15 (IL-15) or a functional fragment thereof, the method comprising (i) genetically editing a population of induced pluripotent stem cells (iPSCs); (ii) generating a monoclonal population of edited iPSCs; and (iii) differentiating said population of iPSCs into a population of Natural Killer (NK) cells.

18. The method of claim 17, wherein step (i) comprises introducing a polynucleotide encoding a soluble form of interleukin-15 (IL-15) or a functional fragment thereof, or a cell membrane-bound form of IL-15 (mbIL-15) or functional fragment thereof, or a mbIL-15 or functional fragment thereof fused to IL-15Rα, or a form of IL-15 trapped in the endoplasmic reticulum (ER).

19. (canceled)

20. (canceled)

21. (canceled)

22. The method of claim 17, wherein the IL-15 or functional fragment thereof is introduced using a TALEN construct.

23. The method of claim 17, wherein the IL-15 is introduced using a Cas9 or Cas12 enzyme.

24. The method of claim 17, wherein the IL-15 is introduced using a Dualase platform.

25. The method of claim 17, wherein step (i) further comprises introducing a deletion in the TGFbR2 gene of the genome of the iPSCs.

26. (canceled)

27. (canceled)

28. A cell population produced by the method of claim 17.

29. A pharmaceutical composition comprising the cell population of claim 1.

30. A method of treating cancer in a subject in need thereof, comprising administering an effective amount of the pharmaceutical composition of claim 29.

31. The method of claim 30, wherein the cancer is a solid tumor or a hematological malignancy.

32. (canceled)

33. A method of inhibiting proliferation of tumor cells in a subject in need thereof, comprising administering an effective amount of the pharmaceutical composition of claim 29.

34. The method of claim 30, wherein the subject is further administered with an immune checkpoint inhibitor.

35. (canceled)

36. The method of any one of claims 30-33 claim 30, wherein the subject is further administered with an NK cell engager.

37. The method of claim 36, wherein the NK cell engager is an NKp46 NK cell engager.

38. The method of claim 30, wherein the subject is further administered with an antibody.
